(19) United States
(12) Patent Application Publication
(10) Pub. No.: US 2023/0257816 A1
(43) Pub. Date: Aug. 17, 2023

(54) METHODS FOR MAKING TREATMENT MANAGEMENT DECISIONS IN TRANSPLANT SUBJECTS AND ASSESSING TRANSPLANT RISKS WITH THRESHOLD VALUES

(71) Applicants: Aoy Tomita MITCHELL, Elm Grove, WI (US); Michael MITCHELL, Elm Grove, WI (US); The Medical College of Wisconsin, Inc., Milwaukee, WI (US)

(72) Inventors: Aoy Tomita Mitchell, Elm Grove, WI (US); Michael Mitchell, Elm Grove, WI (US)

(21) Appl. No.: 18/015,834

(22) PCT Filed: Jul. 12, 2021
(86) PCT No.: PCT/US2021/041341

§ 371 Date: Jan. 12, 2023

Related U.S. Application Data
(60) Provisional application No. 63/051,196, filed on Jul. 13, 2020.
(60) Provisional application No. 63/051,203, filed on Jul. 13, 2020.
(60) Provisional application No. 63/051,343, filed on Jul. 13, 2020.

(51) Int. Cl. C12Q 1/6883; C12Q 1/6851

(52) U.S. Cl. CPC C12Q 1/6883; C12Q 1/6851; C12Q 2600/158; C12Q 2600/118

(57) ABSTRACT

This invention relates to methods and compositions for assessing or monitoring an amount of donor-specific fraction cell-free DNA from a transplant subject and comparing to specific threshold values for making treatment management decisions.

### RELATED APPLICATIONS

This application claims the benefit of priority under 35 U.S.C. § 119(e) to U.S. Provisional Application No. 63/051,196, filed Jul. 13, 2020, U.S. Provisional Application No. 63/051,203, filed Jul. 13, 2020, and U.S. Provisional Application No. 63/051,343, filed Jul. 13, 2020, the entire contents of each of which are incorporated herein by reference.

### FIELD OF THE INVENTION

This invention relates to methods and compositions for assessing an amount of donor-specific cell-free nucleic acids in samples from a transplant subject. Such amounts can be used to monitor a transplant subject to assess risk.

### SUMMARY OF INVENTION

It has been found that donor-specific cf-DNA (DS cf-DNA) is correlated with risk of rejection, such as cellular rejection and/or antibody-mediated rejection. It has also been found that certain threshold values of the DS cf-DNA are particularly meaningful for assessing risk in a transplant subject, such as risk of rejection. Thus, monitoring amounts of these nucleic acids and comparing to these threshold values can be beneficial to assess a transplant subject and allow for any needed intervention. Provided herein are methods, compositions and kits related to such a determination. The methods, compositions, or kits can be any one of the methods, compositions, or kits, respectively, provided herein, including any one of those of the Examples or Figures. In one embodiment of any one of the methods provided, the method further comprises obtaining a sample from the subject.

In one embodiment, any one of the embodiments for the methods provided herein can be an embodiment for any one of the compositions, kits or reports provided. In one embodiment, any one of the embodiments for the compositions, kits or reports provided herein can be an embodiment for any one of the methods provided herein.

In one aspect, a report or database comprising one or more of the amounts provided herein is provided.

In one aspect, a method of treating a subject, determining a treatment regimen for a subject or providing information about a treatment to the subject, based on the amount of donor-specific cell-free DNA or any one of the methods of analysis provided herein is provided. In one embodiment of any one of such methods, the method comprises a step of treating the subject or providing information about a treatment to the subject. In one embodiment of any one of the methods of treating, the treatment may be any one of the treatments provided herein. In one embodiment of any one of the methods of treating, the treatment is for any one of the conditions provided herein. Examples of which are provided herein or otherwise known to those of ordinary skill in the art.

In one aspect, a method of making a treatment management decision for a subject or providing information about a treatment to the subject, based on the amount of donor-specific cell-free DNA or any one of the methods of analysis provided herein is provided. In one embodiment of any one of such methods, the method comprises determining or obtaining the amount of donor-specific cell-free DNA in a sample from the subject taken prior to or at the beginning of treatment for rejection (e.g., Day 0) and/or a sample from the subject at day 14 during a treatment regimen for rejection. In one embodiment of any such method, the amount(s) is compared to a threshold, such as 0.13, 0.14, 0.23, or 0.43, or any one of the values of the below table (Table 1) for day 0 and/or day 14, respectively, such as the mean, median, lower quartile, etc. of the table, respectively, wherein if the amount(s) are greater than or equal to the threshold an increased risk is indicated.

TABLE 1
Threshold Values
Analysis Variable: log2_df

| | Event/No | | | Std | | Lower | Upper | | |
|---|---|---|---|---|---|---|---|---|---|
| group | Event | N | Mean | Dev | Median | Quartile | Quartile | Minimum | Maximum |
| Day 0 | No event | 17 | −1.66 | 1.47 | −1.87 | −2.82 | −1.08 | −3.59 | 1.09 |
| | Event | 5 | 1.48 | 0.87 | 1.18 | 0.97 | 2.11 | 0.51 | 2.65 |
| Day 1 | No event | 16 | −1.46 | 1.41 | −1.47 | −2.69 | −0.24 | −3.60 | 0.66 |
| | Event | 5 | −0.09 | 3.02 | −0.67 | −1.29 | 0.75 | −3.70 | 4.48 |
| Day 4 | No event | 15 | −2.32 | 1.39 | −2.57 | −3.56 | −1.51 | −3.87 | 0.57 |
| | Event | 4 | −2.13 | 2.36 | −2.48 | −4.05 | −0.21 | −4.32 | 0.76 |
| Day 7 | No event | 19 | −1.77 | 1.22 | −1.90 | −2.77 | −0.83 | −3.43 | 1.13 |
| | Event | 5 | −2.00 | 1.51 | −1.51 | −3.26 | −1.24 | −3.82 | −0.17 |
| Day | No event | 21 | −2.68 | 0.98 | −2.84 | −3.22 | −1.90 | −4.35 | −0.82 |
| 14 | Event | 5 | −0.95 | 2.17 | −0.98 | −2.15 | 0.76 | −3.87 | 1.50 |
| Day | No event | 36 | −2.24 | 1.34 | −2.60 | −3.33 | −1.32 | −4.22 | 1.04 |
| 28 | Event | 8 | −1.94 | 1.75 | −2.64 | −3.12 | −0.79 | −3.69 | 1.25 |

In one embodiment, if an increased risk is indicated the method can further comprise making a treatment management decision, such as initiating or changing a treatment in the subject because of the increased indicated risk. The treatment can be a treatment for rejection. The treatment can be a more aggressive treatment. In one embodiment, the more aggressive treatment may be an increase in dosage and/or frequency of the treatment. In one embodiment of any one of such methods, the method comprises a step of treating the subject or providing information about a treatment to the subject. In one embodiment of any one of the methods of treating, the treatment may be any one of the treatments provided herein. In one embodiment of any one of the methods of treating, the treatment is for any one of the conditions provided herein. Examples of which are provided herein or otherwise known to those of ordinary skill in the art.

In one aspect, any one of the methods provided herein may be a method of treating a transplant subject, such as a cardiac transplant subject.

### BRIEF DESCRIPTION OF DRAWINGS

The accompanying drawings are not intended to be drawn to scale. The figures are illustrative only and are not required for enablement of the disclosure.

FIG. 1 illustrates an example of a computer system with which some embodiments may operate.

FIGS. 2A-2C include a graph showing the log 2 donor fraction of samples from all healthy subjects (“0”) and all subjects having acute cellular rejection grades of 2 (ACR2) or more (“1”) (FIG. 2A), and a graph using receiver operating characteristic (ROC) analysis on repeated measures using correlation to examine the relationship between ACR2 or more and donor-fraction cell-free DNA (FIG. 2B). FIG. 2C is a graph showing the experimental determination of a cutpoint (threshold).

FIGS. 3A-3C include a graph showing the log 2 donor fraction of samples from healthy pediatric subjects (“0”) and pediatric subjects having acute cellular rejection grades of 2 (ACR2) or more (“1”) (FIG. 3A), and a graph using receiver operating characteristic (ROC) analysis on repeated measures using correlation to examine the relationship between ACR2 or more and donor-fraction cell-free DNA (FIG. 3B). FIG. 3C is a graph showing the experimental determination of a cutpoint (threshold).

FIGS. 4A-4C include a graph showing the log 2 donor fraction of samples from healthy adult subjects (“0”) and adult subjects having acute cellular rejection grades of 2 (ACR2) or more (“1”) (FIG. 4A), and a graph using receiver operating characteristic (ROC) analysis on repeated measures using correlation to examine the relationship between ACR2 or more and donor-fraction cell-free DNA (FIG. 4B). FIG. 4C is a graph showing the experimental determination of a cutpoint (threshold).

FIGS. 5A-5C include a graph showing the log 2 donor fraction of first samples from subjects (“0”) and subjects having acute cellular rejection grades of 2 (ACR2) or more (“1”) (FIG. 5A), and a graph using receiver operating characteristic (ROC) analysis on repeated measures using correlation to examine the relationship between ACR2 or more and donor-fraction cell-free DNA (FIG. 5B). FIG. 5C is a graph showing the experimental determination of a cutpoint (threshold).

FIGS. 6A-6C include a graph showing the log 2 donor fraction of first samples from pediatric subjects (“0”) and pediatric subjects having acute cellular rejection grades of 2 (ACR2) or more (“1”) (FIG. 6A), and a graph using receiver operating characteristic (ROC) analysis on repeated measures using correlation to examine the relationship between ACR2 or more and donor-fraction cell-free DNA (FIG. 6B). FIG. 6C is a graph showing the experimental determination of a cutpoint (threshold).

FIGS. 7A-7C include a graph showing the log 2 donor fraction of first samples from adult subjects (“0”) and adult subjects having acute cellular rejection grades of 2 (ACR2) or more (“1”) (FIG. 7A), and a graph using receiver operating characteristic (ROC) analysis on repeated measures using correlation to examine the relationship between ACR2 or more and donor-fraction cell-free DNA (FIG. 7B). FIG. 7C is a graph showing the experimental determination of a cutpoint (threshold).

FIGS. 8A-8C include a graph showing the log 2 donor fraction of last samples from subjects (“0”) and subjects having acute cellular rejection grades of 2 (ACR2) or more (“1”) (FIG. 8A), and a graph using receiver operating characteristic (ROC) analysis on repeated measures using correlation to examine the relationship between ACR2 or more and donor-fraction cell-free DNA (FIG. 8B). FIG. 8C is a graph showing the experimental determination of a cutpoint (threshold).

FIGS. 9A-9C include a graph showing the log 2 donor fraction of last samples from pediatric subjects (“0”) and pediatric subjects having acute cellular rejection grades of 2 (ACR2) or more (“1”) (FIG. 9A), and a graph using receiver operating characteristic (ROC) analysis on repeated measures using correlation to examine the relationship between ACR2 or more and donor-fraction cell-free DNA (FIG. 9B). FIG. 9C is a graph showing the experimental determination of a cutpoint (threshold).

FIGS. 10A-10C include a graph showing the log 2 donor fraction of last samples from pediatric subjects (“0”) and pediatric subjects having acute cellular rejection grades of 2 (ACR2) or more (“1”) (FIG. 10A), and a graph using receiver operating characteristic (ROC) analysis on repeated measures using correlation to examine the relationship between ACR2 or more and donor-fraction cell-free DNA (FIG. 10B). FIG. 10C is a graph showing the experimental determination of a cutpoint (threshold).

FIGS. 11A-11C include a graph showing the log 2 donor fraction of samples from all healthy subjects (“0”) and all subjects having antibody-mediated rejection grades of 2 (AMR2) or more (“1”) (FIG. 11A), and a graph using receiver operating characteristic (ROC) analysis on repeated measures using correlation to examine the relationship between AMR2 or more and donor-fraction cell-free DNA (FIG. 11B). FIG. 11C is a graph showing the experimental determination of a cutpoint (threshold).

FIGS. 12A-12C include a graph showing the log 2 donor fraction of samples from healthy pediatric subjects (“0”) and pediatric subjects having antibody-mediated rejection grades of 2 (AMR2) or more (“1”) (FIG. 12A), and a graph using receiver operating characteristic (ROC) analysis on repeated measures using correlation to examine the relationship between AMR2 or more and donor-fraction cell-free DNA (FIG. 12B). FIG. 12C is a graph showing the experimental determination of a cutpoint (threshold).

FIGS. 13A-13C include a graph showing the log 2 donor fraction of samples from healthy adult subjects (“0”) and adult subjects having antibody-mediated rejection grades of 2 (AMR2) or more (“1”) (FIG. 13A), and a graph using receiver operating characteristic (ROC) analysis on repeated measures using correlation to examine the relationship between AMR2 or more and donor-fraction cell-free DNA (FIG. 13B). FIG. 13C is a graph showing the experimental determination of a cutpoint (threshold).

FIGS. 14A-14C include a graph showing the log 2 donor fraction of first samples from subjects (“0”) and subjects having antibody-mediated rejection grades of 2 (AMR2) or more (“1”) (FIG. 14A), and a graph using receiver operating characteristic (ROC) analysis on repeated measures using correlation to examine the relationship between AMR2 or more and donor-fraction cell-free DNA (FIG. 14B). FIG. 14C is a graph showing the experimental determination of a cutpoint (threshold).

FIGS. 15A-15C include a graph showing the log 2 donor fraction of first samples from pediatric subjects (“0”) and pediatric subjects having antibody-mediated rejection grades of 2 (AMR2) or more (“1”) (FIG. 15A), and a graph using receiver operating characteristic (ROC) analysis on repeated measures using correlation to examine the relationship between AMR2 or more and donor-fraction cell-free DNA (FIG. 15B). FIG. 15C is a graph showing the experimental determination of a cutpoint (threshold).

FIGS. 16A-16C include a graph showing the log 2 donor fraction of first samples from adult subjects (“0”) and adult subjects having antibody-mediated rejection grades of 2 (AMR2) or more (“1”) (FIG. 16A), and a graph using receiver operating characteristic (ROC) analysis on repeated measures using correlation to examine the relationship between AMR2 or more and donor-fraction cell-free DNA (FIG. 16B). FIG. 16C is a graph showing the experimental determination of a cutpoint (threshold).

FIGS. 17A-17C include a graph showing the log 2 donor fraction of last samples from subjects (“0”) and subjects having antibody-mediated rejection grades of 2 (AMR2) or more (“1”) (FIG. 17A), and a graph using receiver operating characteristic (ROC) analysis on repeated measures using correlation to examine the relationship between AMR2 or more and donor-fraction cell-free DNA (FIG. 17B). FIG. 17C is a graph showing the experimental determination of a cutpoint (threshold).

FIGS. 18A-18C include a graph showing the log 2 donor fraction of last samples from pediatric subjects (“0”) and pediatric subjects having antibody-mediated rejection grades of 2 (AMR2) or more (“1”) (FIG. 18A), and a graph using receiver operating characteristic (ROC) analysis on repeated measures using correlation to examine the relationship between AMR2 or more and donor-fraction cell-free DNA (FIG. 18B). FIG. 18C is a graph showing the experimental determination of a cutpoint (threshold).

FIGS. 19A-19C include a graph showing the log 2 donor fraction of last samples from pediatric subjects (“0”) and pediatric subjects having antibody-mediated rejection grades of 2 (AMR2) or more (“1”) (FIG. 19A), and a graph using receiver operating characteristic (ROC) analysis on repeated measures using correlation to examine the relationship between AMR2 or more and donor-fraction cell-free DNA (FIG. 19B). FIG. 19C is a graph showing the experimental determination of a cutpoint (threshold).

FIGS. 20A-20C include a graph showing the donor fraction (cell-free DNA) in samples from subjects who were not treated for rejection (“0”) and who were treated for rejection (“1”) (FIG. 20A), and a graph using ROC analysis on repeated measures using correlation to examine the relationship between donor fraction (cell-free DNA) and outcome (treatment for rejection) (FIG. 20B). FIG. 20C is a graph showing the experimental determination of a cutpoint (threshold).

FIGS. 21A-21C include a graph showing the log 2 donor fraction (cell-free DNA) in samples from subjects who were not treated for rejection (“0”) and who were treated for rejection (“1”) (FIG. 21A), and a graph using ROC analysis on repeated measures using correlation to examine the relationship between log 2 donor fraction (cell-free DNA) and outcome (treatment for rejection) (FIG. 21B). FIG. 21C is a graph showing the experimental determination of a cutpoint (threshold).

FIGS. 22A-22C include a graph showing the donor fraction (cell-free DNA) in last samples from subjects who were not treated for rejection (“0”) and who were treated for rejection (“I”) (FIG. 22A), and a graph using ROC analysis on repeated measures using correlation to examine the relationship between donor fraction (cell-free DNA) and outcome (treatment for rejection) (FIG. 22B). FIG. 22C is a graph showing the experimental determination of a cutpoint (threshold).

FIGS. 23A-23C include a graph showing the log 2 donor fraction (cell-free DNA) in last samples from subjects who were not treated for rejection (“0”) and who were treated for rejection (“1”) (FIG. 23A), and a graph using ROC analysis on repeated measures using correlation to examine the relationship between log 2 donor fraction (cell-free DNA) and outcome (treatment for rejection) (FIG. 23B). FIG. 23C is a graph showing the experimental determination of a cutpoint (threshold).

FIGS. 24A-24C include a graph showing the donor fraction (cell-free DNA) in first samples from subjects who were not treated for rejection (“0”) and who were treated for rejection (“1”) (FIG. 24A), and a graph using ROC analysis on repeated measures using correlation to examine the relationship between donor fraction (cell-free DNA) and outcome (treatment for rejection) (FIG. 24B). FIG. 24C is a graph showing the experimental determination of a cutpoint (threshold).

FIGS. 25A-25C include a graph showing the log 2 donor fraction (cell-free DNA) in first samples from subjects who were not treated for rejection (“0”) and who were treated for rejection (“1”) (FIG. 25A), and a graph using ROC analysis on repeated measures using correlation to examine the relationship between log 2 donor fraction (cell-free DNA) and outcome (treatment for rejection) (FIG. 25B). FIG. 25C is a graph showing the experimental determination of a cutpoint (threshold).

FIGS. 26A-26B are scatterplots showing the log 2 donor-fraction cell-free DNA present in samples from subjects who were alive with no post-T0 event (narrow rings) or dead or alive with at least one post-T0 event (thick rings). FIG. 26A shows the results from all the samples; FIG. 26B shows the results from samples taken up to and including T10 (ten days after treatment for rejection). In both plots, “0” represents Time 0 (T0).

FIG. 27 is a boxplot showing log 2 donor-fraction cell-free DNA present in samples from subjects who were alive with no post-T0 event or dead or alive with at least one post-T0 event at different time points. Day “0” represents Time 0 (T0).

FIGS. 28A-28C show plots of log 2 donor-fraction cell-free DNA over time for subjects who died. The far right vertical line represents death. The other vertical lines represent onset of mechanical circulatory support (MCS) in FIGS. 28A and 28B. In FIG. 28C, the left vertical line represents cardiac arrest (CA). In all plots, “0” represents Time 0.

FIGS. 29A-29D show plots of log 2 donor-fraction cell-free DNA over time for subjects who survived and experienced cardiac arrest and/or required MCS. In FIGS. 29A-29C, the vertical lines represent onset of MCS. In FIG. 29D, the vertical lines represent cardiac arrest. In all plots, “0” represents Time 0.

FIGS. 30A-30K show plots of log 2 donor-fraction cell-free DNA over time for subjects who survived and did not experience cardiac arrest or require MCS.

FIG. 31 includes two graphs showing the distribution of donor-fraction (DF) values between healthy samples and treatment for rejection samples (right graph) and the experimental determination of a cutpoint (right graph). All samples were analyzed.

FIG. 32 includes two graphs showing the distribution of donor-fraction (DF) values between healthy samples and treatment for rejection samples (right graph) and the experimental determination of a cutpoint (right graph). Pediatric samples were analyzed.

FIG. 33 includes two graphs showing the distribution of donor-fraction (DF) values between healthy samples and treatment for rejection samples (right graph) and the experimental determination of a cutpoint (right graph). Adult samples were analyzed.

FIG. 34 includes two graphs showing the distribution of donor-fraction (DF) values between healthy samples and treatment for rejection samples (right graph) and the experimental determination of a cutpoint (right graph). All first samples were used.

FIG. 35 includes two graphs showing the distribution of donor-fraction (DF) values between healthy samples and treatment for rejection samples (right graph) and the experimental determination of a cutpoint (right graph). Pediatric first samples were used.

FIG. 36 includes two graphs showing the distribution of donor-fraction (DF) values between healthy samples and treatment for rejection samples (right graph) and the experimental determination of a cutpoint (right graph). Adult first samples were used.

FIG. 37 includes two graphs showing the distribution of donor-fraction (DF) values between healthy samples and treatment for rejection samples (right graph) and the experimental determination of a cutpoint (right graph). All last samples were used.

FIG. 38 includes two graphs showing the distribution of donor-fraction (DF) values between healthy samples and treatment for rejection samples (right graph) and the experimental determination of a cutpoint (right graph). Pediatric last samples were used.

FIG. 39 includes two graphs showing the distribution of donor-fraction (DF) values between healthy samples and treatment for rejection samples (right graph) and the experimental determination of a cutpoint (right graph). Adult last samples were used.

FIG. 40 is boxplot comparing Time 0 donor-fraction cell-free DNA values versus subsequent days post-treatment for rejection.

FIG. 41 is a boxplot comparing donor-fraction cell-free DNA values across event or no event post-treatment for rejection.

### DETAILED DESCRIPTION OF THE INVENTION

As used herein, “donor-specific nucleic acids” (or “donor fraction nucleic acids”) refers to nucleic acids that are from a transplant donor that can be found in a transplant recipient. Such nucleic acids are preferably cell-free DNA. “Cell-free DNA” (or “cf-DNA”) is DNA that is present outside of a cell, e.g., in the blood, plasma, serum, urine, etc. of a subject. As used herein, the compositions and methods provided herein can be used to determine an amount of DS cf-DNA. As used herein, “transplant” refers to an organ or tissue moved from a donor to a recipient for the purpose of replacing the recipient's damaged or absent organ or tissue. Any one of the methods or compositions provided herein may be used on a sample from a subject that has undergone a transplant of an organ or tissue. In some embodiments of any one of the methods provided herein, the transplant is a heart transplant. In some embodiments of any one of the methods provided herein, the heart transplant subject is an adult transplant subject. In some embodiments of any one of the methods provided herein, the heart transplant subject is pediatric transplant subject.

Amounts of DS cf-DNA can be used to assess or determine risk or prognosis of transplant rejection. As provided herein, any one of the methods can be used to assess a subject that has or is suspected of having cellular rejection and/or antibody-mediated rejection. As used herein, “suspected of having” refers to a subject whereby a clinician believes there is a likelihood the subject has a specific condition, such as transplant rejection. The methods provided herein can be used to confirm a finding of rejection or monitor such a subject for worsening or improving rejection condition.

As provided herein, any one of the methods can be used to assess a subject that has or is suspected of having a transplant complication. In one embodiment of any one of the methods provided herein, the subject may be one that has a transplant complication or that a clinician believes there is a likelihood of having a transplant complication. In some embodiments, any one of the methods can be used to assess a subject that has had or is at risk of having a transplant complication. Subjects may be suspected of having, determined to have had, or determined to have a likelihood or risk of having a transplant complication based on symptoms (and/or lack thereof). However, in some embodiments, the subject is suspected of having, determined to have had, or determined to have a likelihood or risk of having a transplant complication based on one or more other tests. In such an embodiment, the methods provided herein can be used to confirm such a finding or monitor such a subject for worsening or improving condition.

An amount of cf-DNA (DS and/or total) may be determined with experimental techniques, such as those provided elsewhere herein. “Obtaining” as used herein refers to any method by which the respective information or materials can be acquired. Thus, the respective information can be acquired by experimental methods. Respective materials can be created, designed, etc. with various experimental or laboratory methods, in some embodiments. The respective information or materials can also be acquired by being given or provided with the information, such as in a report, or materials. Materials may be given or provided through commercial means (i.e. by purchasing), in some embodiments.

Because of the ability to determine amounts of nucleic acids, such as cf-DNA, and the correlation with transplant conditions, the methods and compositions provided herein can be used to assess subjects. Thus, a risk of improving or worsening rejection condition can be determined in such subjects. A “risk” as provided herein, refers to the presence or absence or progression of any undesirable condition in a subject, or an increased likelihood of the presence or absence or progression of such a condition. As provided herein “increased risk” refers to the presence or progression of any undesirable condition in a subject or an increased likelihood of the presence or progression of such a condition. As provided herein, “decreased risk” refers to the absence of any undesirable condition or progression in a subject or a decreased likelihood of the presence or progression (or increased likelihood of the absence or non-progression) of such a condition. In any one of the methods provided herein, the condition is rejection.

As provided herein, early detection or monitoring can facilitate treatment and improve clinical outcomes. Any one of the methods provided can be performed on any one of the subjects provided herein. Such methods can be used to monitor a subject over time, with or without treatment. Further, such methods can aid in the selection, administration and/or monitoring of a treatment or therapy. Accordingly, the methods provided herein can be used to determine a treatment or monitoring regimen.

“Determining a treatment regimen”, as used herein, refers to the determination of a course of action for treatment of the subject. In one embodiment of any one of the methods provided herein, determining a treatment regimen includes determining an appropriate therapy or information regarding an appropriate therapy to provide to a subject. In some embodiments of any one of the methods provided herein, the determining includes providing an appropriate therapy or information regarding an appropriate therapy to a subject. As used herein, information regarding a treatment or therapy or monitoring may be provided in written form or electronic form. In some embodiments, the information may be provided as computer-readable instructions. In some embodiments, the information may be provided orally.

“Making a treatment management decision”, as used herein, refers to any decision a clinician may make for a subject as provided herein in order to monitor and/or treat the subject, such as one with transplant rejection. Treatment management decisions include, but are not limited to, determining that additional testing and/or monitoring is required, initiating a treatment, changing the frequency of a treatment, changing the dosage of the treatment, changing the frequency and/or dosage of the treatment, changing the type of treatment to be performed, changing the timing of the treatment, or any combination of the foregoing. In any one of the methods provided herein, the step of making a treatment management decision can include any one or more of the foregoing. In any one of the methods provided herein, the method may comprise or further comprise a step of treating or monitoring the subject (or recommending the treatment or monitoring to the subject) according to the treatment management decision.

The therapies can be, for example, for treating rejection, such as an anti-rejection therapy. Anti-rejection therapies include, for example, immunosuppressives. Immunosuppressives include, but are not limited to, corticosteroids (e.g., prednisolone or hydrocortisone), glucocorticoids, cytostatics, alkylating agents (e.g., nitrogen mustards (cyclophosphamide), nitrosoureas, platinum compounds, cyclophosphamide (Cytoxan)), antimetabolites (e.g., folic acid analogues, such as methotrexate, purine analogues, such as azathioprine and mercaptopurine, pyrimidine analogues, and protein synthesis inhibitors), cytotoxic antibiotics (e.g., dactinomycin, anthracyclines, mitomycin C, bleomycin, mithramycin), antibodies (e.g., anti-CD20, anti-IL-1, anti-IL-2Ralpha, anti-T-cell or anti-CD-3 monoclonals and polyclonals, such as Atgam, and Thymoglobuline), drugs acting on immunophilins, ciclosporin, tacrolimus, sirolimus, interferons, opioids, TNF-binding proteins, mycophenolate, fingolimod and myriocin. In some embodiments, anti-rejection therapy comprises blood transfer or marrow transplant. Therapies can also include intravenous fluids, antibiotics, surgical drainage, early goal directed therapy (EGDT), vasopressors, steroids, activated protein C, drotrecogin alfa (activated), oxygen and appropriate support for organ dysfunction. This may include hemodialysis in kidney failure, mechanical ventilation in pulmonary dysfunction, transfusion of blood products, and drug and fluid therapy for circulatory failure. Ensuring adequate nutrition—preferably by enteral feeding, but if necessary, by parenteral nutrition—can also be included particularly during prolonged illness. Other associated therapies can include insulin and medication to prevent deep vein thrombosis and gastric ulcers.

The therapies can be, for example, for treating antibody-mediated rejection. Antibody-mediated rejection therapies include, for example, immunosuppressives, plasmapheresis/plasma exchange, intravenous immunoglobulin, corticosteroids, anti-lymphocyte antibodies, and splenectomy.

Other such therapies are known to those of ordinary skill in the art.

Administration of a treatment or therapy may be accomplished by any method known in the art (see, e.g., Harrison's Principle of Internal Medicine, McGraw Hill Inc.). Preferably, administration of a treatment or therapy occurs in a therapeutically effective amount. Administration may be local or systemic. Administration may be parenteral (e.g., intravenous, subcutaneous, or intradermal) or oral. Compositions for different routes of administration are known in the art (see, e.g., Remington's Pharmaceutical Sciences by E. W. Martin).

The treatment and clinical course may be determined by the subject's expected risk as provided herein. For example, if the amount of DS cf-DNA is equal to 0.13 or 0.14 or greater, risk or rejection is indicated. As another example, if the amount of DS cf-DNA is equal to or greater than a threshold, such as 0.13, 0.14, 0.23, or 0.43, or any one of the values of Table 1 for day 0 and/or day 14, respectively, such as the mean, median, lower quartile, etc. of the table, respectively, risk or rejection is indicated, and the subject may be treated with, or provided information related thereto, anti-rejection therapies, such as those described above.

“Determining a monitoring regimen”, as used herein, refers to determining a course of action to monitor a condition in the subject over time. In one embodiment of any one of the methods provided herein, determining a monitoring regimen includes determining an appropriate course of action for determining the amount of DS cf-DNA in the subject over time or at a subsequent point in time, or suggesting such monitoring to the subject. This can allow for the measurement of variations in a clinical state and/or permit calculation of normal values or baseline levels (as well as comparisons thereto). In some embodiments of any one of the methods provided herein determining a monitoring regimen includes determining the timing and/or frequency of obtaining samples from the subject and/or determining or obtaining an amount of DS cf-DNA.

In some embodiments, amounts of DS cf-DNA can be plotted over time. In some embodiments, threshold values for the points in time may also be plotted. A comparison with a subject's cf-DNA levels to threshold values over a period of time can be used to predict risk.

As increasing levels of DS cf-DNA have been found to correlate with an increased risk of transplant complications, a clinician may determine that a subject should undergo more frequent sampling if the subject's DS cf-DNA are found to increase between time points. If a subject is found to have decreasing levels of DS cf-DNA between time points, a clinician may determine that less frequent sampling is sufficient. Accordingly, if a subject does not show such a decrease, the clinician may determine that additional testing and/or treatment may be necessary. Timing and/or frequency of monitoring may also be determined by a comparison to threshold values. For example, if the amount of DS cf-DNA is equal to or greater than a threshold, such as 0.13, 0.14, 0.23, or 0.43, or any one of the values in Table 1 for day 0 and/or day 14, respectively, such as the mean, median, lower quartile, etc. of the table, respectively, and/or is increasing, more frequent sampling may be needed, whereas, if the amount of DS cf-DNA is less than the threshold, such as 0.13, 0.14, 0.23, or 0.43, or any one of the values of Table 1 for day 0 and/or day 14, respectively, such as the mean, median, lower quartile, etc. of the table, respectively, and/or is not increasing, less frequent sampling may be required.

In some embodiments of any one of the methods provided herein, each amount and time point may be recorded in a report or in a database. Threshold values may also be recorded in a report or in a database.

Reports with any one or more of the values as provided herein are also provided in an aspect. Reports may be in oral, written (or hard copy) or electronic form, such as in a form that can be visualized or displayed. Preferably, the report provides the amount of donor-specific nucleic acids in a sample. In some embodiments, the report provides amounts of donor-specific nucleic acids in samples from a subject over time, and can further include corresponding threshold values in some embodiments.

In some embodiments, the amounts and/or threshold values are in or entered into a database. In one aspect, a database with such amounts and/or values is provided. From the amount(s), a clinician may assess the need for a treatment or monitoring of a subject. Accordingly, in any one of the methods provided herein, the method can include assessing the amount of nucleic acids in the subject at more than one point in time. Such assessing can be performed with any one of the methods or compositions provided herein.

As used herein, “amount” refers to any quantitative value for the measurement of nucleic acids and can be given in an absolute or relative amount. Further, the amount can be a total amount, frequency, ratio, percentage, etc. As used herein, the term “level” can be used instead of “amount” but is intended to refer to the same types of values. Generally, unless otherwise provided, the amounts provided herein represent the ratio or percentage, when referring to DS cf-DNA, in a sample relative to the total.

In some embodiments, any one of the methods provided herein can comprise comparing an amount of donor-specific nucleic acids to a threshold value to identify a subject at increased or decreased risk. In some embodiments of any one of the methods provided herein, a subject having an increased amount of nucleic acids compared to a threshold value is identified as being at increased risk. In some embodiments of any one of the methods provided herein, a subject having a decreased or similar amount of nucleic acids compared to a threshold value is identified as being at decreased or not increased risk.

“Threshold” or “threshold value” or “cutpoint” or “cutoff”, as used herein, refers to any predetermined level or range of levels that is indicative of the presence or absence of a condition or the presence or absence of a risk. The threshold value can take a variety of forms. It can be single cut-off value, such as a median or mean. As another example, a threshold value can be determined from baseline values before the presence of a condition or risk or before or after a course of treatment. Such a baseline can be indicative of a normal or other state in the subject not correlated with the risk or condition that is being tested for. In some embodiments, the threshold value can be a baseline value of the subject being tested. The threshold value of any one of the methods, reports, databases, etc. provided herein, can be any one of the threshold values provided herein, such as 0.13, 0.14, 0.23, or 0.43, or any one of the values of Table 1 for day 0 and/or day 14, respectively, such as the mean, median, lower quartile, etc. of the table, respectively.

The threshold values can be used for comparisons to make treatment and/or monitoring decisions. For example, if the amount of DS cf-DNA is equal to or greater than a threshold, such as 0.13, 0.14, 0.23, or 0.43, or any one of the values of Table 1 for day 0 and/or day 14, respectively, such as the mean, median, lower quartile, etc. of the table, respectively, and/or increasing over time, further monitoring may be indicated. As a further example, if the amount is equal to or greater than a threshold, such as 0.13, 0.14, 0.23, or 0.43, or any one of the values of Table 1 for day 0 and/or day 14, respectively, such as the mean, median, lower quartile, etc. of the table, respectively, treatment of the subject may be indicated. If the amount is equal to or greater a threshold, such as 0.13, 0.14, 0.23, or 0.43, or any one of the values of Table 1 for day 0 and/or day 14, respectively, such as the mean, median, lower quartile, etc. of the table, respectively, additional testing of the subject, such as with a biopsy may be indicated.

The threshold values provided herein can be used to determine the presence or absence of rejection, or risk associated therewith, in the subject, in some embodiments. Accordingly, if the amount of DS cf-DNA measured is less than a threshold, such as 0.13, 0.14, 0.23, or 0.43, or any one of the values of Table 1 for day 0 and/or day 14, respectively, such as the mean, median, lower quartile, etc. of the table, respectively, the subject may not have or be at risk of rejection. If the amount is equal to or greater than a threshold, such as 0.13, 0.14, 0.23, or 0.43, or any one of the values of Table 1 for day 0 and/or day 14, respectively, such as the mean, median, lower quartile, etc. of the table, respectively, then the subject may have or be at risk of rejection. The determination of the presence or absence of rejection can be done based on any one of the comparisons as provided herein with or without other indicators of such a condition.

In any one of the methods provided herein, the threshold is 0.09. In any one of the methods provided herein, the threshold is 0.10. In any one of the methods provided herein, the threshold is 0.12. In any one of the methods provided herein, the threshold is 0.13. In any one of the methods provided herein, the threshold is 0.14. In any one of the methods provided herein, the threshold is 0.15. In any one of the methods provided herein, the threshold is 0.16. In any one of the methods provided herein, the threshold is 0.17. In any one of the methods provided herein, the threshold is 0.18. In any one of the methods provided herein, the threshold is 0.19. In any one of the methods provided herein, the threshold is 0.20. In any one of the methods provided herein, the threshold is 0.21. In any one of the methods provided herein, the threshold is 0.22. In any one of the methods provided herein, the threshold is 0.23. In any one of the methods provided herein, the threshold is 0.24. In any one of the methods provided herein, the threshold is 0.25. In any one of the methods provided herein, the threshold is 0.26. In any one of the methods provided herein, the threshold is 0.27. In any one of the methods provided herein, the threshold is 0.28. In any one of the methods provided herein, the threshold is 0.29. In any one of the methods provided herein, the threshold is 0.30. In any one of the methods provided herein, the threshold is 0.31. In any one of the methods provided herein, the threshold is 0.32. In any one of the methods provided herein, the threshold is 0.33. In any one of the methods provided herein, the threshold is 0.34. In any one of the methods provided herein, the threshold is 0.35. In any one of the methods provided herein, the threshold is 0.36. In any one of the methods provided herein, the threshold is 0.37. In any one of the methods provided herein, the threshold is 0.38. In any one of the methods provided herein, the threshold is 0.39. In any one of the methods provided herein, the threshold is 0.40. In any one of the methods provided herein, the threshold is 0.41. In any one of the methods provided herein, the threshold is 0.42. In any one of the methods provided herein, the threshold is 0.43. In any one of the methods provided herein, the threshold is 0.44. In any one of the methods provided herein, the threshold is 0.45. In any one of the methods provided herein, the threshold is 0.46. In any one of the methods provided herein, the threshold is 0.47. In any one of the methods provided herein, the threshold is 0.48. In any one of the methods provided herein, the threshold is 0.49. In any one of the methods provided herein, the threshold is 0.50.

In any one of the methods provided herein, the subject is a pediatric subject (i.e., age at transplant was less than 18 years of age), and the threshold is 0.197, 0.13, 0.22, 0.43, or 0.86.

In any one of the methods provided herein, the subject is an adult subject (i.e., age at transplant was 18 years of age or greater), and threshold is 0.098, 0.16, 0.17, 0.19, or 0.34.

Any one of the methods provided herein may further include an additional test(s) for assessing the subject, or a step of suggesting such further testing to the subject (or providing information about such further testing). The additional test(s) may be any one of the methods provided herein. The additional test(s) may be any one of the other methods provided herein or otherwise known in the art as appropriate.

The amount of cf-DNA may be determined by a number of methods. In some embodiments such a method is a sequencing-based method. In one embodiment, any one of the methods for determining cf-DNA may be any one of the methods of U.S. Publication No. 2015-0086477-A1, and such methods are incorporated herein by reference in their entirety. An amount of cf-DNA may also be determined by a MOMA assay. In one embodiment, any one of the methods for determining cf-DNA may be any one of the methods of PCT Publication No. WO 2016/176662 A1, and such methods are incorporated herein by reference in their entirety.

As used herein, the sample from a subject can be a biological sample. Examples of such biological samples include whole blood, plasma, serum, urine, etc.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different from illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The following description provides examples of the methods provided herein.

### EXAMPLES

### Example 1—Examples of Computer-Implemented Embodiments

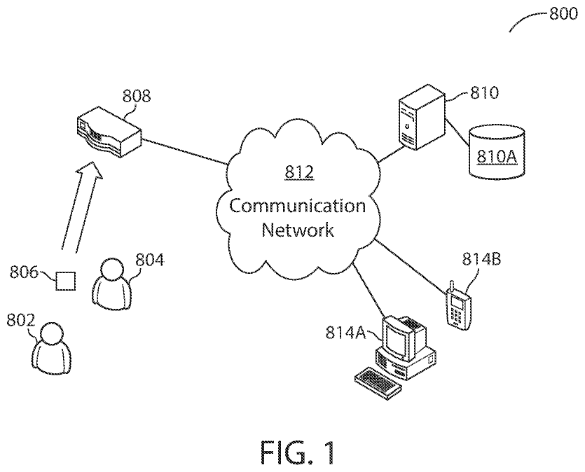

In some embodiments, the diagnostic techniques described above may be implemented via one or more computing devices executing one or more software facilities to analyze samples for a subject, such as over time, measure nucleic acids (such as cell-free DNA) in the samples, and produce a result, such as a diagnostic result, based on one or more of the samples. FIG. 1 illustrates an example of a computer system with which some embodiments may operate, though it should be appreciated that embodiments are not limited to operating with a system of the type illustrated in FIG. 1.

The computer system of FIG. 1 includes a subject 802 and a clinician 804 that may obtain a sample 806 from the subject 806. As should be appreciated from the foregoing, the sample 806 may be any suitable sample of biological material for the subject 802 that may be used to measure the presence of nucleic acids (such as cell-free DNA) in the subject 802, including a blood sample. The sample 806 may be provided to an analysis device 808, which one of ordinary skill will appreciate from the foregoing will analyze the sample 808 so as to determine (including estimate) amounts of nucleic acids (such as cell-free DNA), including amounts of DS nucleic acids (such as DS cell-free DNA) in the sample 806 and/or the subject 802. For ease of illustration, the analysis device 808 is depicted as single device, but it should be appreciated that analysis device 808 may take any suitable form and may, in some embodiments, be implemented as multiple devices. To determine the amounts of nucleic acids (such as cell-free DNA) in the sample 806 and/or subject 802, the analysis device 808 may perform any of the techniques described above, and is not limited to performing any particular analysis. The analysis device 808 may include one or more processors to execute an analysis facility implemented in software, which may drive the processor(s) to operate other hardware and receive the results of tasks performed by the other hardware to determine on overall result of the analysis, which may be the amounts of nucleic acids (such as cell-free DNA) in the sample 806 and/or the subject 802. The analysis facility may be stored in one or more computer-readable storage media, such as a memory of the device 808. In other embodiments, techniques described herein for analyzing a sample may be partially or entirely implemented in one or more special-purpose computer components such as Application Specific Integrated Circuits (ASICs), or through any other suitable form of computer component that may take the place of a software implementation.

In some embodiments, the clinician 804 may directly provide the sample 806 to the analysis device 808 and may operate the device 808 in addition to obtaining the sample 806 from the subject 802, while in other embodiments the device 808 may be located geographically remote from the clinician 804 and subject 802 and the sample 806 may need to be shipped or otherwise transferred to a location of the analysis device 808. The sample 806 may in some embodiments be provided to the analysis device 808 together with (e.g., input via any suitable interface) an identifier for the sample 806 and/or the subject 802, for a date and/or time at which the sample 806 was obtained, or other information describing or identifying the sample 806.

The analysis device 808 may in some embodiments be configured to provide a result of the analysis performed on the sample 806 to a computing device 810, which may include a data store 810A that may be implemented as a database or other suitable data store. The computing device 810 may in some embodiments be implemented as one or more servers, including as one or more physical and/or virtual machines of a distributed computing platform such as a cloud service provider. In other embodiments, the device 810 may be implemented as a desktop or laptop personal computer, a smart mobile phone, a tablet computer, a special-purpose hardware device, or other computing device.

In some embodiments, the analysis device 808 may communicate the result of its analysis to the device 810 via one or more wired and/or wireless, local and/or wide-area computer communication networks, including the Internet. The result of the analysis may be communicated using any suitable protocol and may be communicated together with the information describing or identifying the sample 806, such as an identifier for the sample 806 and/or subject 802 or a date and/or time the sample 806 was obtained.

The computing device 810 may include one or more processors to execute a diagnostic facility implemented in software, which may drive the processor(s) to perform diagnostic techniques described herein. The diagnostic facility may be stored in one or more computer-readable storage media, such as a memory of the device 810. In other embodiments, techniques described herein for analyzing a sample may be partially or entirely implemented in one or more special-purpose computer components such as Application Specific Integrated Circuits (ASICs), or through any other suitable form of computer component that may take the place of a software implementation.

The diagnostic facility may receive the result of the analysis and the information describing or identifying the sample 806 and may store that information in the data store 810A. The information may be stored in the data store 810A in association with other information for the subject 802, such as in a case that information regarding prior samples for the subject 802 was previously received and stored by the diagnostic facility. The information regarding multiple samples may be associated using a common identifier, such as an identifier for the subject 802. In some cases, the data store 810A may include information for multiple different subjects.

The diagnostic facility may also be operated to analyze results of the analysis of one or more samples 806 for a particular subject 802, identified by user input, so as to determine a diagnosis for the subject 802. The diagnosis may be a conclusion of a risk that the subject 802 has, may have, or may in the future develop a particular condition. The diagnostic facility may determine the diagnosis using any of the various examples described above, including by comparing the amounts of nucleic acids (such as cell-free DNA) determined for a particular sample 806 to one or more thresholds or by comparing a change over time in the amounts of nucleic acids (such as cell-free DNA) determined for samples 806 over time, such as to one or more thresholds. For example, the diagnostic facility may determine a risk to the subject 802 of a condition by comparing an amount of nucleic acids (such as cell-free DNA) for one or more samples 806 to one threshold and comparing an amount of nucleic acids (such as cell-free DNA) for the same sample(s) 806 to another threshold. Based on the comparisons to the thresholds, the diagnostic facility may produce an output indicative of a risk to the subject 802 of a condition.

As should be appreciated from the foregoing, in some embodiments, the diagnostic facility may be configured with different thresholds to which amounts of nucleic acids (such as cell-free DNA) may be compared. The different thresholds may, for example, correspond to different demographic groups (age, gender, race, economic class, presence or absence of a particular procedure/condition/other in medical history, or other demographic categories), different conditions, and/or other parameters or combinations of parameters. In such embodiments, the diagnostic facility may be configured to select thresholds against which amounts of nucleic acids (such as cell-free DNA) are to be compared, with different thresholds stored in memory of the computing device 810. The selection may thus be based on demographic information for the subject 802 in embodiments in which thresholds differ based on demographic group, and in these cases demographic information for the subject 802 may be provided to the diagnostic facility or retrieved (from another computing device, or a data store that may be the same or different from the data store 810A, or from any other suitable source) by the diagnostic facility using an identifier for the subject 802. The selection may additionally or alternatively be based on the condition for which a risk is to be determined, and the diagnostic facility may prior to determining the risk receive as input a condition and use the condition to select the thresholds on which to base the determination of risk. It should be appreciated that the diagnostic facility is not limited to selecting thresholds in any particular manner, in embodiments in which multiple thresholds are supported.

In some embodiments, the diagnostic facility may be configured to output for presentation to a user a user interface that includes a diagnosis of a risk and/or a basis for the diagnosis for a subject 802. The basis for the diagnosis may include, for example, amounts of nucleic acids (such as cell-free DNA) detected in one or more samples 806 for a subject 802. In some embodiments, user interfaces may include any of the examples of results, values, amounts, graphs, etc. discussed above. They can include results, values, amounts, etc. over time. For example, in some embodiments, a user interface may incorporate a graph similar to that shown in any one of the figures provided herein. In such a case, in some cases the graph may be annotated to indicate to a user how different regions of the graph may correspond to different diagnoses that may be produced from an analysis of data displayed in the graph. For example, thresholds against which the graphed data may be compared to determine the analysis may be imposed on the graph(s).

A user interface including a graph, particularly with the lines and/or shading, may provide a user with a far more intuitive and faster-to-review interface to determine a risk of the subject 802 based on amounts of nucleic acids (such as cell-free DNA), than may be provided through other user interfaces. It should be appreciated, however, that embodiments are not limited to being implemented with any particular user interface.

In some embodiments, the diagnostic facility may output the diagnosis or a user interface to one or more other computing devices 814 (including devices 814A, 814B) that may be operated by the subject 802 and/or a clinician, which may be the clinician 804 or another clinician. The diagnostic facility may transmit the diagnosis and/or user interface to the device 814 via the network(s) 812.

Techniques operating according to the principles described herein may be implemented in any suitable manner. Included in the discussion above are a series of flow charts showing the steps and acts of various processes that determine a risk of a condition based on an analysis of amounts of nucleic acids (such as cell-free DNA). The processing and decision blocks discussed above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that embodiments are not limited to any particular syntax or operation of any particular circuit or of any particular programming language or type of programming language. Rather, one skilled in the art may use the description above to fabricate circuits or to implement computer software algorithms to perform the processing of a particular apparatus carrying out the types of techniques described herein. It should also be appreciated that, unless otherwise indicated herein, the particular sequence of steps and/or acts described above is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as a portion of a computing device or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 1, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more Field-Programmable Gate Arrays (FPGAs) for carrying out the techniques described herein, or any other suitable system.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Any one of the aforementioned, including the aforementioned devices, systems, embodiments, methods, techniques, algorithms, media, hardware, software, interfaces, processors, displays, networks, inputs, outputs or any combination thereof are provided herein in other aspects.

### Example 2—Rejection (Acute Cellular Rejection Grade 2 or Higher) Compared to Healthy Samples (ALU Ratio Cutoff=0.50)

In this Example, a healthy sample is defined as one in which the subject has an acute cellular rejection grade (ACR) of 0 and an antibody-mediated rejection (AMR) grade of 0 or the subject has an ACR of 0 and the AMR was not reported. Note that only samples having total cell-free DNA and donor-specific cell-free DNA (donor fraction, DF) were used for analysis.

The exclusion criteria were as follows:

Exclusion criteria

| | |
|---|---|
| 1. Clinical exclusions | Treated for rejection in the last 28 days (samples within |
| | 28 days post treatment for rejection) |
| | Less than 8 days post-transplant (samples within 8 days |
| | post-transplant) |
| | Pregnant |
| | Have another transplanted organ, including bone |
| | marrow (anyone with such case) |
| | Have post-transplant lymphoproliferative disease (if |
| | they ever had it) - anyone with PTLD |
| | Have cancer or have had cancer in the previous 2 years |
| | (anyone with such case) |
| | Mechanical Circulatory Support at the time of |
| | collection (samples drawn during the procedure) |
| 2. Pre- and post-Genotype | TCF, alu ratio and DF with any “QC fail” |
| Quality Control exclusions | |
| Exclusions for healthy controls | Samples within 30 days of death |
| (any one of these criteria) | Samples within 30 days of cardiac arrest (±30 days) |
| The healthy control is defined as: | Samples associated to CAV (graft vasculopathy) |
| ACR grade 0 and AMR grade 0 | Samples taken within 14 days prior to initiation of |
| or | treatment for infection and 3 weeks after initiation. |
| ACR grade 0 and AMR not | Samples during MCS and 1 week after |
| reported | Samples within 30 days of * time zero (±30 days) |
| | Samples within 30 days of ** positive cardiac biopsy |
| | (±30 days) |

* time zero is onset of treatment for new episode of rejection, if any two treatment happened within 30 days, keep the first treatment
** positive cardiac biopsy is defined as biopsy results with ACR grade 1+ and/or AMR grade 1+, or either one of them with positive rejection but couldn't report rejection grade.
Undecided CR should be excluded
Undecided AMR not excluded unless CR is excluded

Initially, the data from 1582 samples (147 subjects) was subjected to the exclusion criteria noted above, resulting in 759 samples from 130 subjects. Of those samples, 373 samples from 117 subjects were analyzed, including 147 samples from 57 pediatric subjects (age at transplant <18 years old) and 226 samples from 60 adult subjects (age at transplant ±18). Of the 117 subjects, 53 pediatric subjects and 44 adult subjects were in the healthy group (total, 97 subjects) and 4 pediatric subjects and 16 adult subjects in the test group (acute cellular rejection grade 2 or higher) (total, 20 subjects).

### Statistical Methods

Generalized linear models for repeated measures were used (subjects as cluster using covariance structures as appropriate) with logit link function to model the probability of rejection. The acr2pB, the log2_df was used to fit the model. The predicted values from the models were used to create the receiver operating characteristics (ROCs).

First and last sample analyses were done using a logistic model, as first/last sample implies the first/last sample related to the outcome for the test group and the first/last healthy sample from rest of the subjects during their participation in the study. ROCs were created for both repeated and one sample cases.

An Alu ratio threshold of 0.50 was used for all results described herein.

### Summary of Results

The following table presents the results from the analysis, which are described in more detail below.

| | p-value | AUC | Sens. | Spec. | PPV | NPV | Cutoff |
|---|---|---|---|---|---|---|---|
| All samples (repeated measure) | <0.0001 | 0.71 | 0.57 | 0.79 | 0.18 | 0.96 | 0.14 |
| All samples (Peds) | 0.002 | 0.89 | 0.75 | 0.97 | 0.38 | 0.99 | 0.86 |
| All samples (Adult) | 0.007 | 0.75 | 0.54 | 0.89 | 0.36 | 0.94 | 0.14 |
| First sample | 0.010 | 0.66 | 0.65 | 0.64 | 0.27 | 0.90 | 0.13 |
| First sample (Peds) | 0.006 | 0.87 | 0.75 | 0.96 | 0.6 | 0.98 | 0.86 |
| First sample (Adult) | 0.035 | 0.66 | 0.56 | 0.80 | 0.5 | 0.83 | 0.14 |
| Last sample | 0.014 | 0.68 | 0.55 | 0.82 | 0.39 | 0.90 | 0.15 |
| Last sample (Peds) | 0.005 | 0.91 | 0.75 | 0.96 | 0.6 | 0.98 | 0.83 |
| Last sample (Adult) | 0.044 | 0.71 | 0.63 | 0.80 | 0.53 | 0.85 | 0.098 |

Note that the Sensitivity/Specificity/PPV/NPV/Cutoff reported in the table above are at the optimum point (i.e., largest of sensitivity + specificity).

### Results

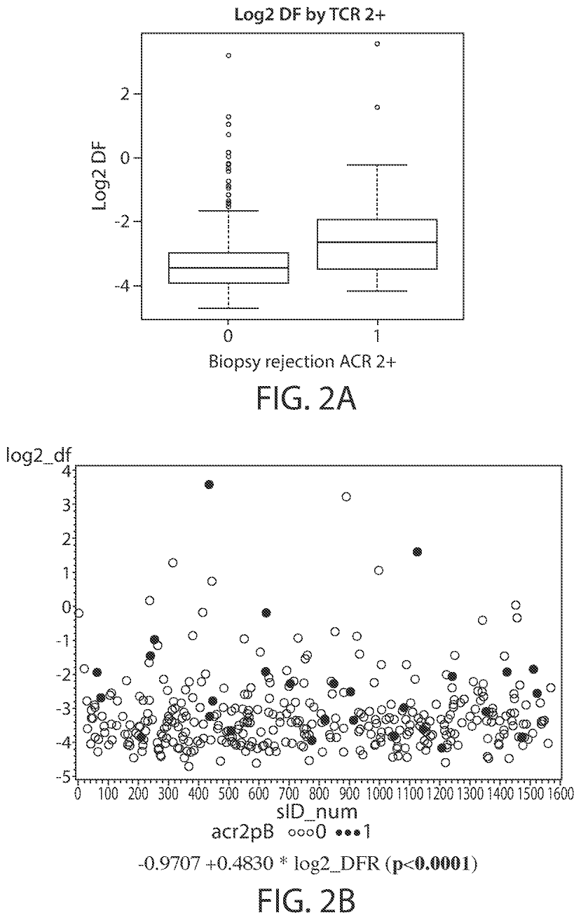

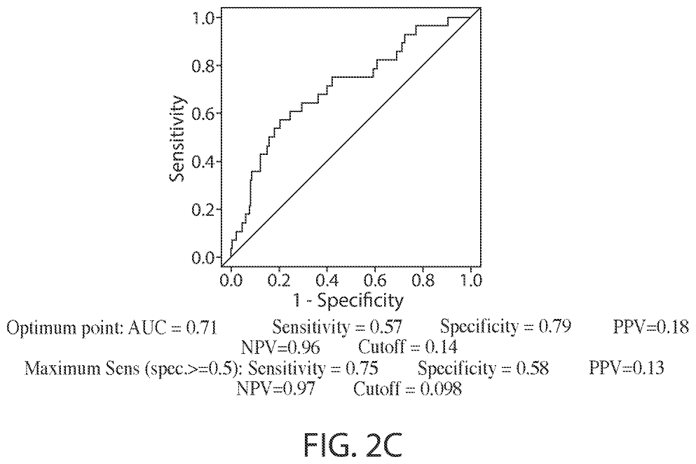

First, rejection (defined as ACR2 or greater) was compared to donor-fraction cell-free DNA (all samples). The results are shown in FIGS. 2A-2C and the table below.

| | N | | | | Std | | Lower | Upper | | |
|---|---|---|---|---|---|---|---|---|---|---|
| Status | Obs | Variable | N | Mean | Dev | Median | Quartile | Quartile | Minimum | Maximum |
| Healthy | 345 | df | 345 | 0.17 | 0.54 | 0.09 | 0.07 | 0.13 | 0.04 | 9.20 |
| | | log2_df | 345 | −3.26 | 1.00 | −3.44 | −3.91 | −2.99 | −4.71 | 3.20 |
| ACR2 or | 28 | df | 28 | 0.71 | 2.24 | 0.16 | 0.09 | 0.26 | 0.06 | 11.81 |
| greater | | log2_df | 28 | −2.33 | 1.70 | −2.64 | −3.48 | −1.93 | −4.17 | 3.56 |

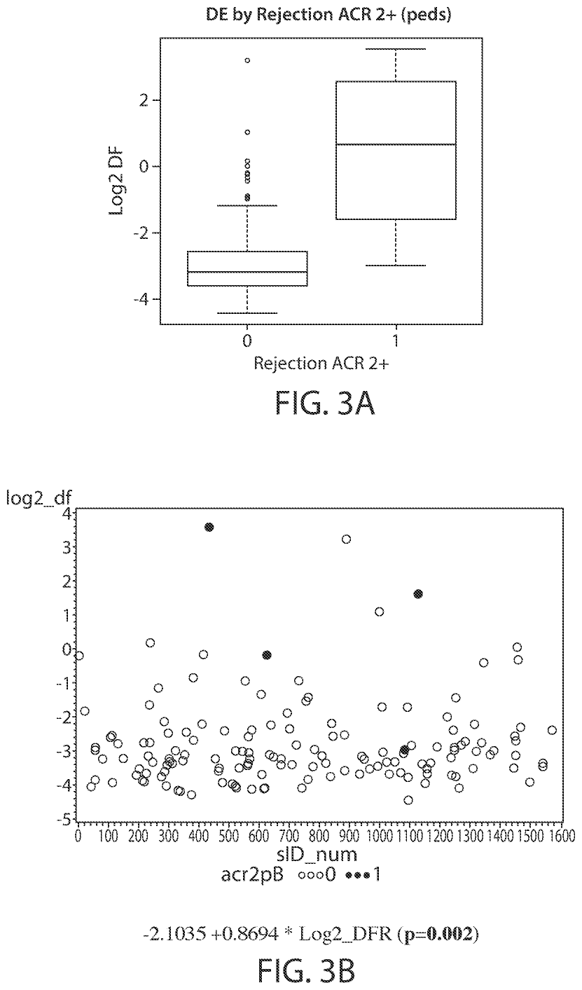

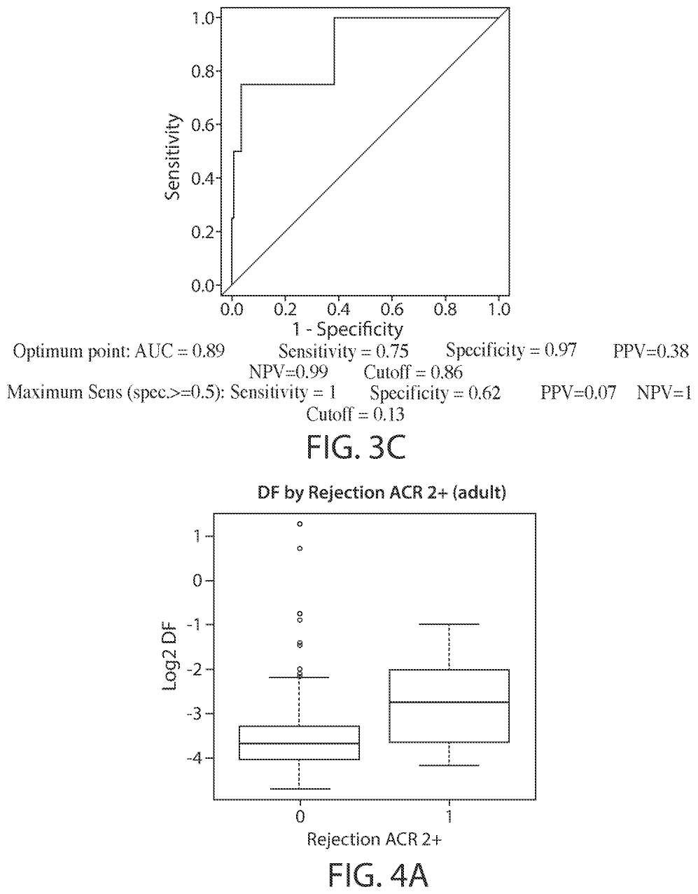

Rejection (defined as ACR2 or greater) was compared to donor-fraction cell-free DNA among pediatric subjects. The results are shown in FIGS. 3A-3C and the table below.

| | N | | | | Std | | Lower | Upper | | |
|---|---|---|---|---|---|---|---|---|---|---|
| acr2pB | Obs | Variable | N | Mean | Dev | Median | Quartile | Quartile | Minimum | Maximum |
| 0 | 143 | df | 143 | 0.25 | 0.79 | 0.11 | 0.08 | 0.17 | 0.05 | 9.20 |
| | | log2_df | 143 | −2.89 | 1.13 | −3.17 | −3.60 | −2.54 | −4.44 | 3.20 |
| 1 | 4 | df | 4 | 3.95 | 5.38 | 1.94 | 0.50 | 7.41 | 0.13 | 11.81 |
| | | log2_df | 4 | 0.49 | 2.78 | 0.69 | −1.59 | 2.57 | −2.97 | 3.56 |

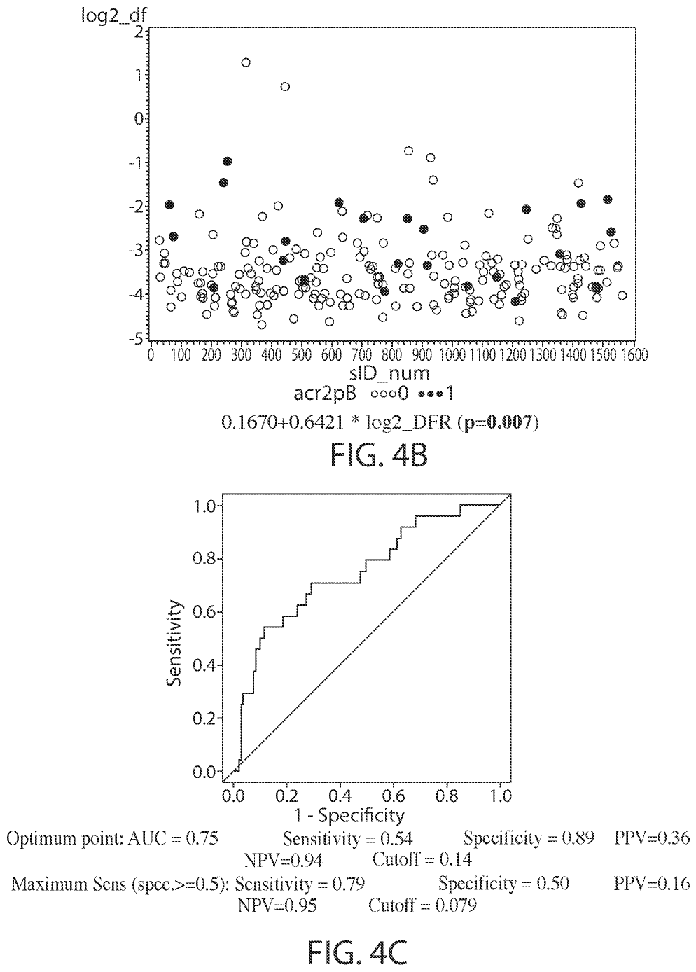

Rejection (defined as ACR2 or greater) was compared to donor-fraction cell-free DNA among adult subjects. The results are shown in FIGS. 4A-4C and the table below.

| | N | | | | Std | | Lower | Upper | | |
|---|---|---|---|---|---|---|---|---|---|---|
| acr2pB | Obs | Variable | N | Mean | Dev | Median | Quartile | Quartile | Minimum | Maximum |
| 0 | 202 | df | 202 | 0.12 | 0.21 | 0.08 | 0.06 | 0.10 | 0.04 | 2.42 |
| | | log2_df | 202 | −3.52 | 0.80 | −3.68 | −4.03 | −3.28 | −4.71 | 1.28 |
| 1 | 24 | df | 24 | 0.17 | 0.11 | 0.15 | 0.08 | 0.25 | 0.06 | 0.51 |
| | | log2_df | 24 | −2.80 | 0.89 | −2.74 | −3.64 | −2.02 | −4.17 | — |

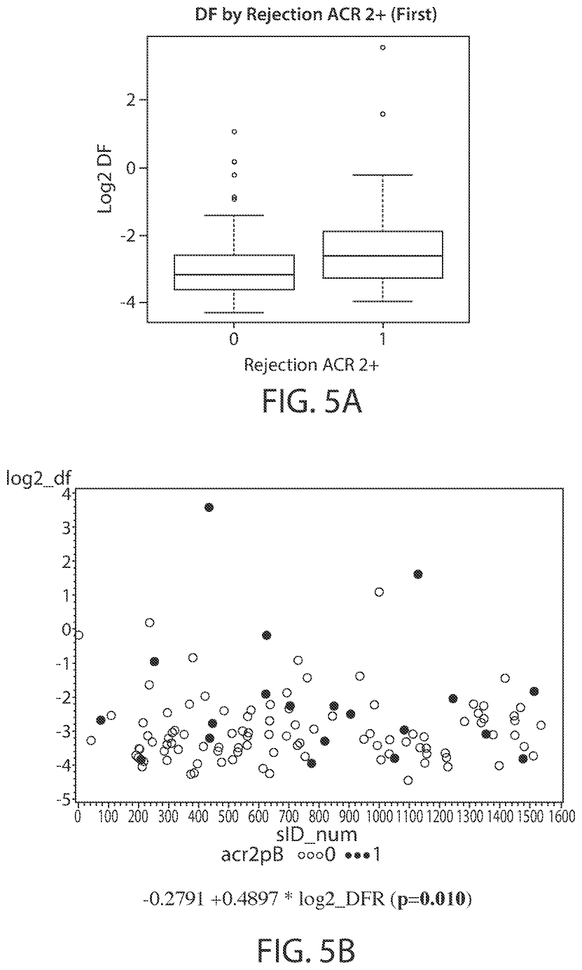

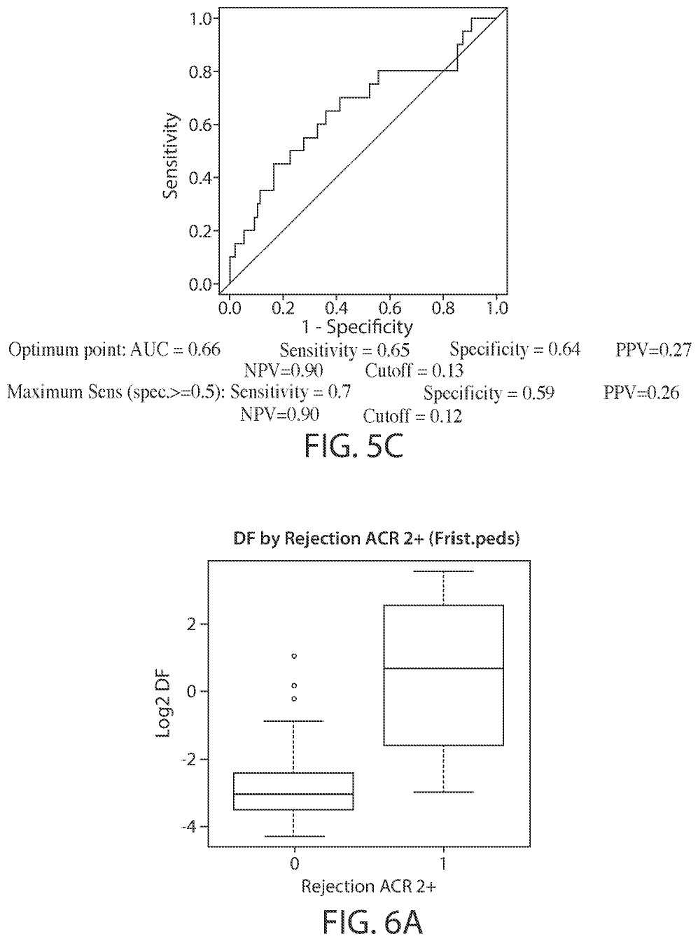

Rejection (defined as ACR2 or greater) was compared to donor-fraction cell-free DNA using the first sample from all subjects. The results are shown in FIGS. 5A-5C and the table below.

| | N | | | | Std | | Lower | Upper | | |
|---|---|---|---|---|---|---|---|---|---|---|
| acr2pB | Obs | Variable | N | Mean | Dev | Median | Quartile | Quartile | Minimum | Maximum |
| 0 | 97 | df | 97 | 0.17 | 0.25 | 0.11 | 0.08 | 0.17 | 0.05 | 2.08 |
| | | log2_df | 97 | −3.00 | 0.95 | −3.16 | −3.60 | −2.59 | −4.29 | 1.05 |
| 1 | 20 | df | 20 | 0.93 | 2.64 | 0.16 | 0.10 | 0.27 | 0.06 | 11.81 |
| | | log2_df | 20 | −2.13 | 1.90 | −2.60 | −3.27 | −1.89 | −3.95 | 3.56 |

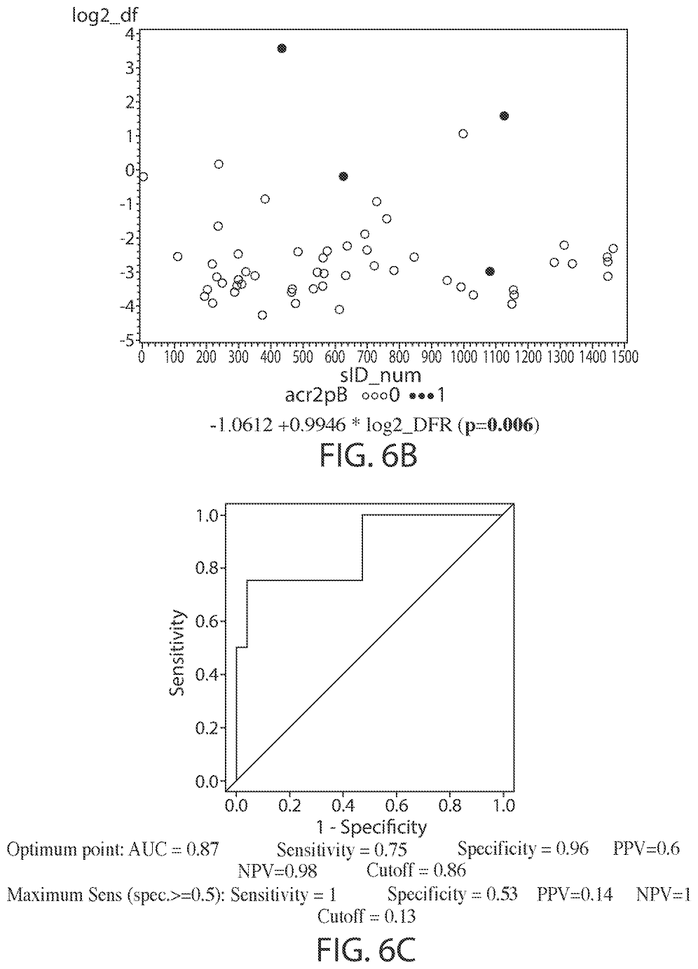

Rejection (defined as ACR2 or greater) was compared to donor-fraction cell-free DNA using the first sample from pediatric subjects. The results are shown in FIGS. 6A-6C and the table below.

| | N | | | | Std | | Lower | Upper | | |
|---|---|---|---|---|---|---|---|---|---|---|
| acr2pB | Obs | Variable | N | Mean | Dev | Median | Quartile | Quartile | Minimum | Maximum |
| 0 | 53 | df | 53 | 0.22 | 0.32 | 0.12 | 0.09 | 0.19 | 0.05 | 2.08 |
| | | log2_df | 53 | −2.77 | 1.07 | −3.03 | −3.50 | −2.41 | −4.29 | 1.05 |
| 1 | 4 | df | 4 | 3.95 | 5.38 | 1.94 | 0.50 | 7.41 | 0.13 | 11.81 |
| | | log2_df | 4 | 0.49 | 2.78 | 0.69 | −1.59 | 2.57 | −2.97 | 3.56 |

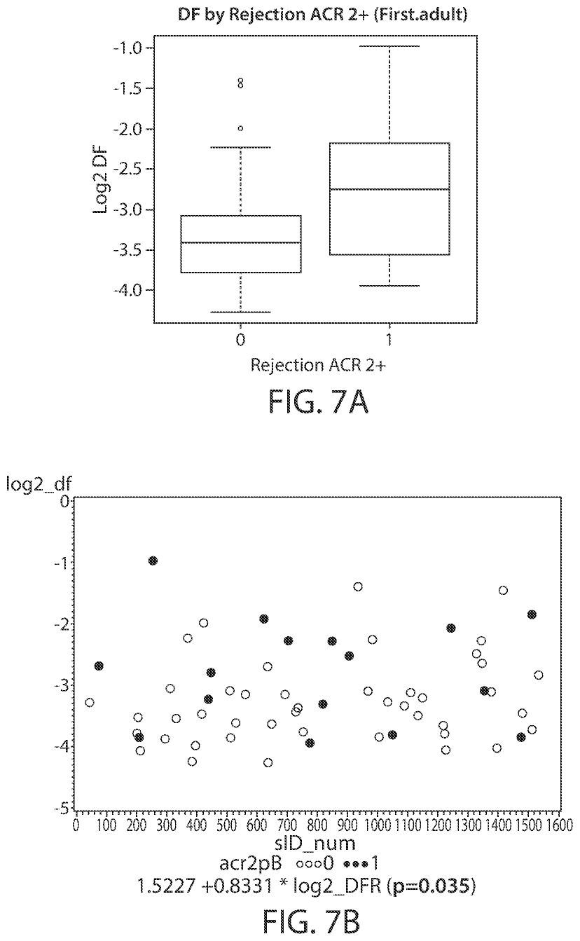

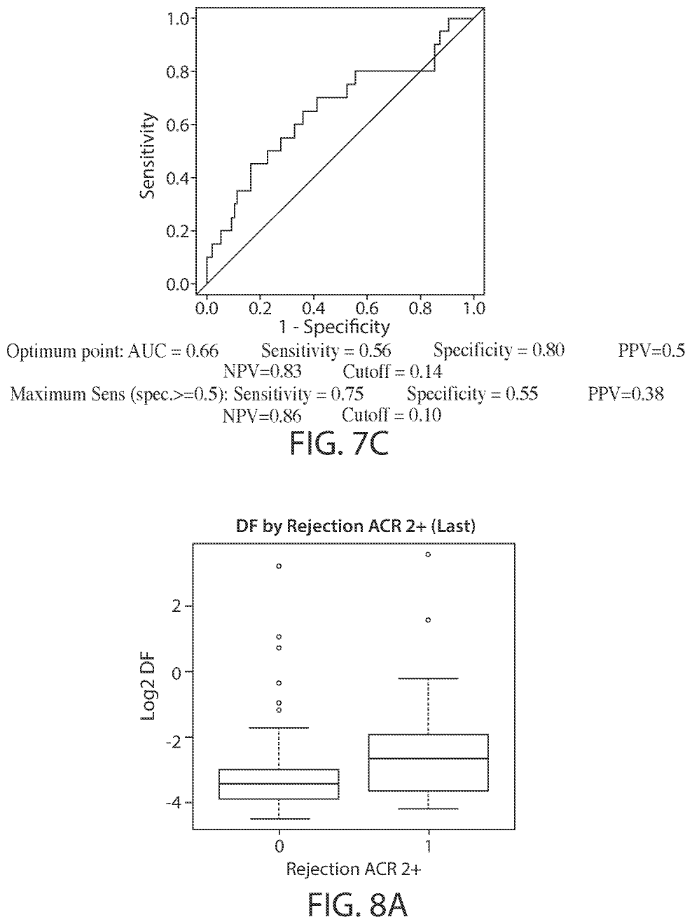

Rejection (defined as ACR2 or greater) was compared to donor-fraction cell-free DNA using the first sample from adult subjects. The results are shown in FIGS. 7A-7C and the table below.

| | N | | | | Std | | Lower | Upper | | |
|---|---|---|---|---|---|---|---|---|---|---|
| acr2pB | Obs | Variable | N | Mean | Dev | Median | Quartile | Quartile | Minimum | Maximum |
| 0 | 44 | df | 44 | 0.12 | 0.07 | 0.09 | 0.07 | 0.12 | 0.05 | 0.38 |
| | | log2_df | 44 | −3.27 | 0.69 | −3.40 | −3.78 | −3.07 | −4.27 | −1.40 |
| 1 | 16 | df | 16 | 0.17 | 0.11 | 0.15 | 0.09 | 0.22 | 0.06 | 0.51 |
| | | log2_df | 16 | −2.78 | 0.87 | −2.74 | −3.56 | −2.18 | −3.95 | −0.98 |

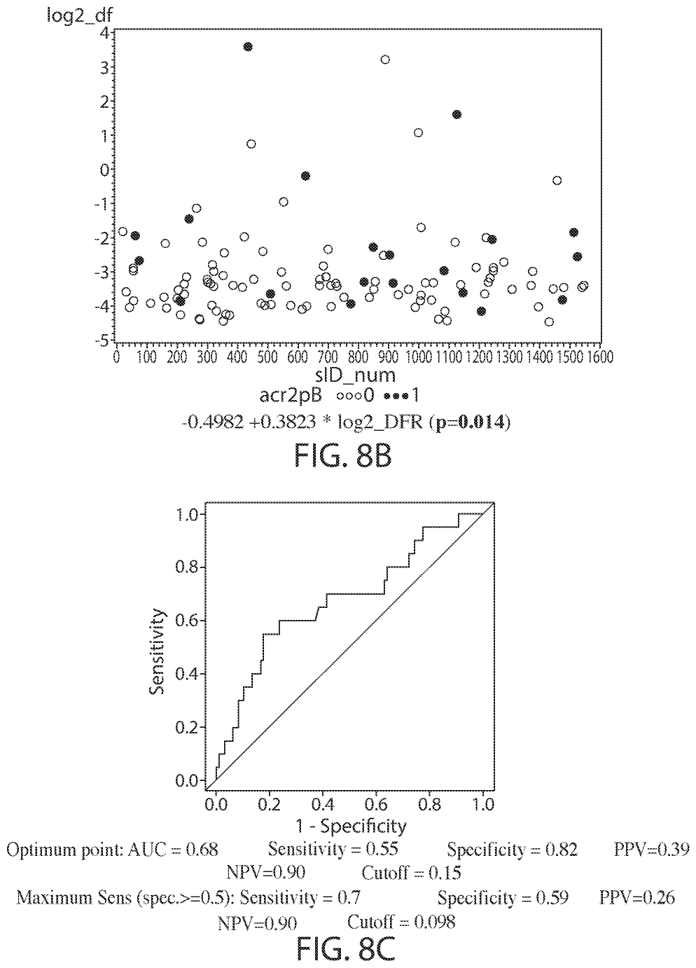

Rejection (defined as ACR2 or greater) was compared to donor-fraction cell-free DNA using the last sample from all subjects. The results are shown in FIGS. 8A-8C and the table below.

| | N | | | | Std | | Lower | Upper | | |
|---|---|---|---|---|---|---|---|---|---|---|
| acr2pB | Obs | Variable | N | Mean | Dev | Median | Quartile | Quartile | Minimum | Maximum |
| 0 | 97 | df | 97 | 0.25 | 0.96 | 0.09 | 0.07 | 0.13 | 0.04 | 9.20 |
| | | log2_df | 97 | −3.20 | 1.18 | −3.42 | −3.88 | −2.99 | −4.49 | 3.20 |
| 1 | 20 | df | 20 | 0.91 | 2.65 | 0.16 | 0.08 | 0.27 | 0.06 | 11.81 |
| | | log2_df | 20 | −2.26 | 1.95 | −2.64 | −3.64 | −1.90 | −4.17 | 3.56 |

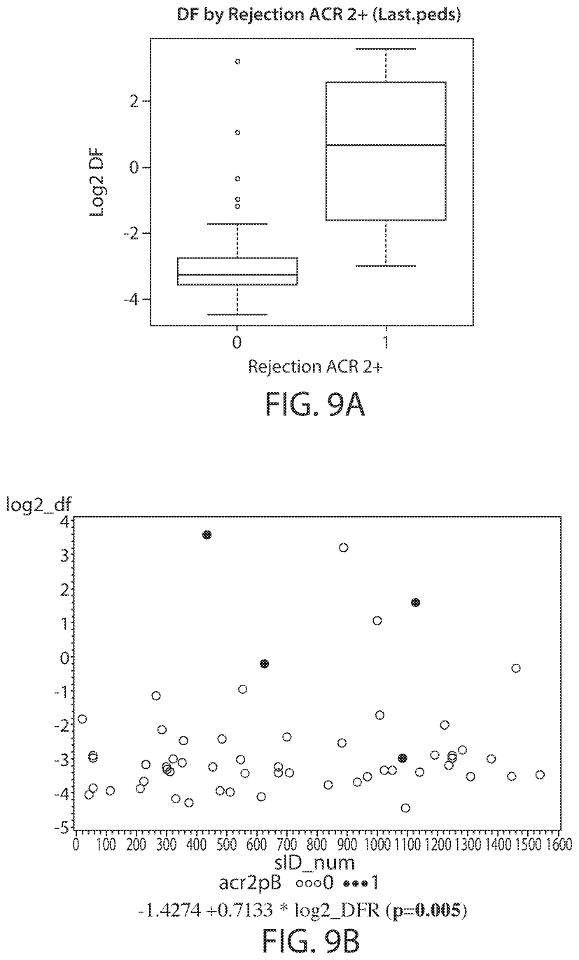

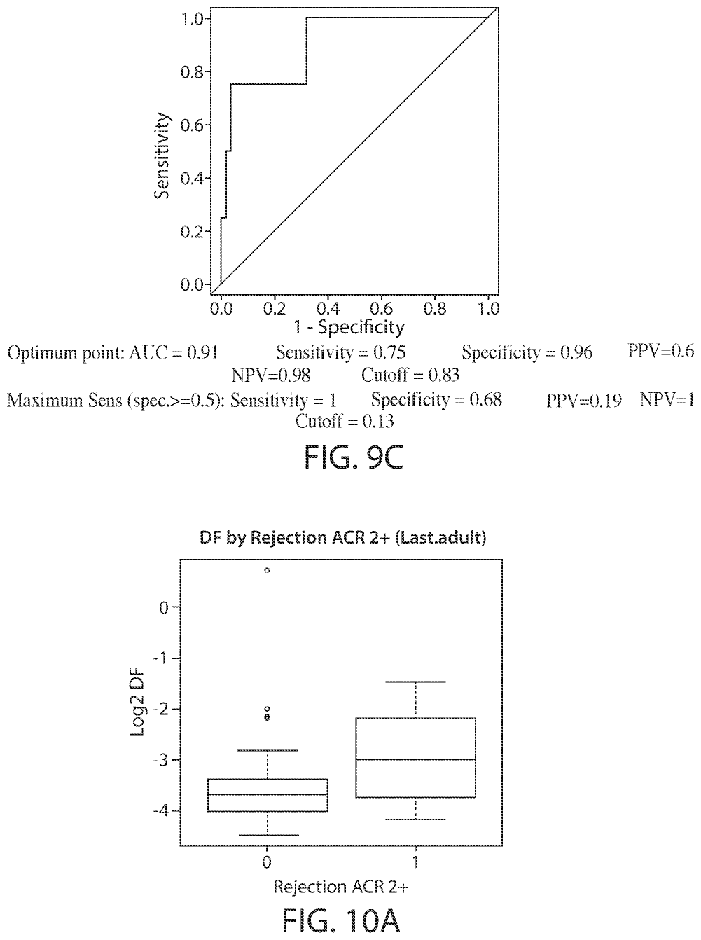

Rejection (defined as ACR2 or greater) was compared to donor-fraction cell-free DNA using the last sample from pediatric subjects. The results are shown in FIGS. 9A-9C and the table below.

| | N | | | | Std | | Lower | Upper | | |
|---|---|---|---|---|---|---|---|---|---|---|
| acr2pB | Obs | Variable | N | Mean | Dev | Median | Quartile | Quartile | Minimum | Maximum |
| 0 | 53 | df | 53 | 0.35 | 1.27 | 0.11 | 0.09 | 0.15 | 0.05 | 9.20 |
| | | log2_df | 53 | −2.91 | 1.32 | −3.23 | −3.53 | −2.73 | −4.44 | 3.20 |
| 1 | 4 | df | 4 | 3.95 | 5.38 | 1.94 | 0.50 | 7.41 | 0.13 | 11.81 |
| | | log2_df | 4 | 0.49 | 2.78 | 0.69 | −1.59 | 2.57 | −2.97 | 3.56 |

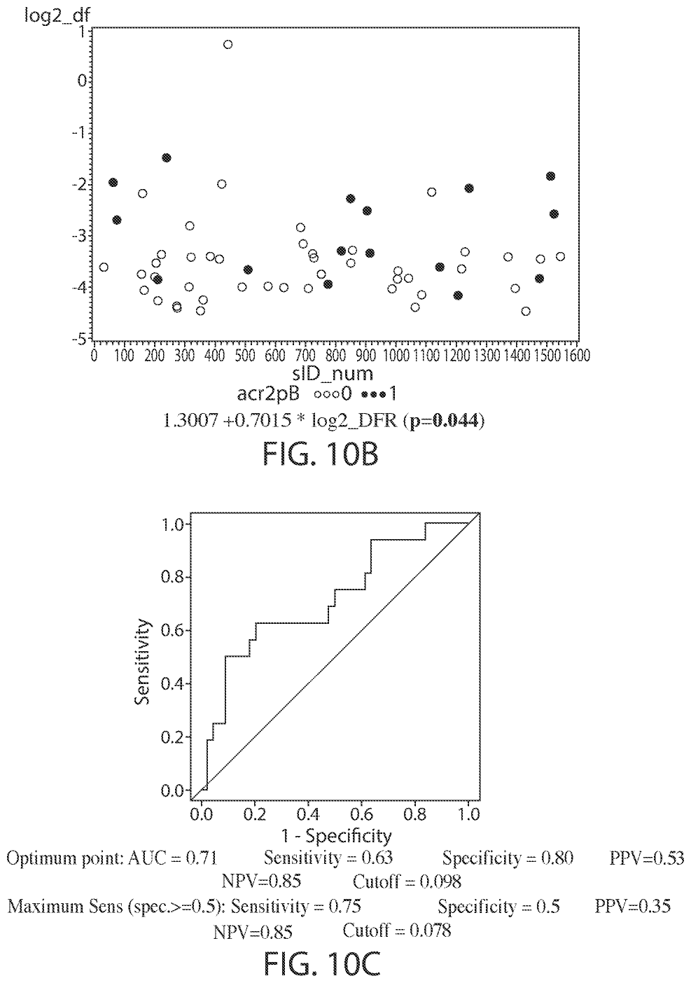

Rejection (defined as ACR2 or greater) was compared to donor-fraction cell-free DNA using the last sample from adult subjects. The results are shown in FIGS. 10A-10C and the table below.

| | N | | | | Std | | Lower | Upper | | |
|---|---|---|---|---|---|---|---|---|---|---|
| acr2pB | Obs | Variable | N | Mean | Dev | Median | Quartile | Quartile | Minimum | Maximum |
| 0 | 44 | df | 44 | 0.12 | 0.24 | 0.08 | 0.06 | 0.10 | 0.04 | 1.66 |
| | | log2_df | 44 | −3.54 | 0.88 | −3.68 | −4.03 | −3.39 | −4.49 | 0.73 |
| 1 | 16 | df | 16 | 0.15 | 0.09 | 0.13 | 0.07 | 0.22 | 0.06 | 0.36 |
| | | log2_df | 16 | −2.95 | 0.87 | −3.00 | −3.76 | −2.18 | −4.17 | −1.47 |

### Example 3—Rejection (Antibody-Mediated Rejection Grade 2 or Higher) Compared to Healthy Samples (ALU Ratio Cutoff=0.50)

In this Example, a healthy sample is defined as one in which the subject has an acute cellular rejection grade (ACR) of 0 and an antibody-mediated rejection (AMR) grade of 0 or the subject has an ACR of 0 and the AMR was not reported. Note that only samples having total cell-free DNA and donor-specific cell-free DNA (donor fraction, DF) were used for analysis.

The exclusion criteria were as follows:

Exclusion criteria

| | |
|---|---|
| 1. Clinical exclusions | Treated for rejection in the last 28 days (samples within |
| | 28 days post treatment for rejection) |
| | Less than 8 days post-transplant (samples within 8 days |
| | post-transplant) |
| | Pregnant |
| | Have another transplanted organ, including bone |
| | marrow (anyone with such case) |
| | Have post-transplant lymphoproliferative disease (if |
| | they ever had it) - anyone with PTLD |
| | Have cancer or have had cancer in the previous 2 years |
| | (anyone with such case) |
| | Mechanical Circulatory Support at the time of |
| | collection (samples drawn during the procedure) |
| 2. Pre- and post-Genotype | TCF, alu ratio and DF with any “QC fail” |
| Quality Control exclusions | |
| Exclusions for healthy controls | Samples within 30 days of death |
| (any one of these criteria) | Samples within 30 days of cardiac arrest (±30 days) |
| The healthy control is defined as | Samples associated to CAV (graft vasculopathy) |
| ACR grade 0 and AMR grade 0 | Samples taken within 14 days prior to initiation of |
| | treatment for infection and 3 weeks after initiation. |
| | Samples during MCS and 1 week after |
| | Samples within 30 days of * time zero (±30 days) |
| | Samples within 30 days of ** positive cardiac |
| | biopsy (±30 days) |

* time zero is onset of treatment for new episode of rejection, if any two treatment happened within 30 days, keep the first treatment
** positive cardiac biopsy is defined as biopsy results with ACR grade 1+ and/or AMR grade 1+, or either one of them with positive rejection but couldn't report rejection grade.
Undecided CR should be excluded
Undecided AMR not excluded unless CR is excluded

Initially, the data from 1582 samples (147 subjects) was subjected to the exclusion criteria noted above, resulting in 759 samples from 130 subjects. Of those samples, 376 samples from 116 subjects were analyzed, including 161 samples from 59 pediatric subjects (age at transplant <18 years old) and 215 samples from 57 adult subjects (age at transplant ±18). Of the 116 subjects, 49 pediatric subjects and 49 adult subjects were in the healthy group (total, 98 subjects) and 10 pediatric subjects and 8 adult subjects in the test group (antibody-mediated rejection grade 2 or higher) (total, 18 subjects).

### Statistical Methods

Generalized linear models for repeated measures were used (subjects as cluster using covariance structures as appropriate) with logit link function to model the probability of rejection. The amr2pB, the log2_df as used to fit the model. The predicted values from the models were used to create the receiver operating characteristics (ROCs).

First and last sample analyses were done using a logistic model, as first/last sample implies the first/last sample related to the outcome for the test group and the first/last healthy sample from rest of the subjects during their participation in the study. ROCs were created for both repeated and one sample cases.

An Alu ratio threshold of 0.50 was used for all results described herein.

### Summary of Results

The following table presents the results from the analysis, which are described in more detail below.

| | p-value | AUC | Sens. | Spec. | PPV | NPV | Cutoff |
|---|---|---|---|---|---|---|---|
| All samples (repeated measure) | <0.0001 | 0.84 | 0.74 | 0.84 | 0.29 | 0.97 | 0.17 |
| All samples (Peds) | 0.023 | 0.77 | 0.67 | 0.80 | 0.3 | 0.95 | 0.197 |
| All samples (Adult) | <0.0001 | 0.88 | 0.77 | 0.92 | 0.37 | 0.98 | 0.17 |
| First sample | 0.002 | 0.74 | 0.61 | 0.84 | 0.41 | 0.92 | 0.20 |
| First sample (Peds) | 0.29 | 0.65 | 0.50 | 0.84 | 0.38 | 0.89 | 0.22 |
| First sample (Adult) | 0.006 | 0.81 | 0.75 | 0.88 | 0.5 | 0.96 | 0.19 |
| Last sample | 0.001 | 0.77 | 0.67 | 0.83 | 0.41 | 0.93 | 0.15 |
| Last sample (Peds) | 0.085 | 0.69 | 0.7 | 0.67 | 0.30 | 0.92 | 0.13 |
| Last sample (Adult) | 0.007 | 0.83 | 0.75 | 0.90 | 0.55 | 0.96 | 0.16 |

Note that the Sensitivity/Specificity/PPV/NPV/Cutoff reported in the table above are at the optimum point (i.e., largest of sensitivity + specificity).

### Results

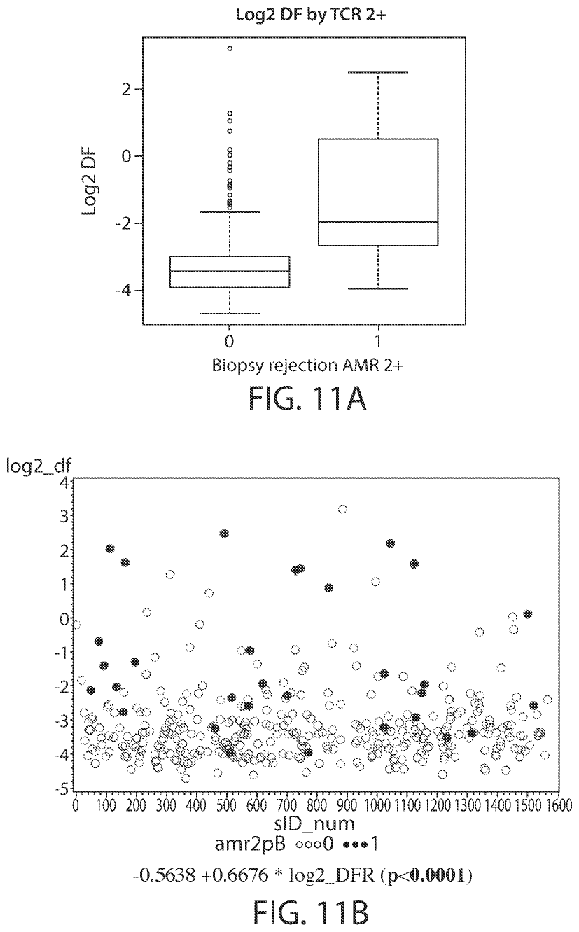

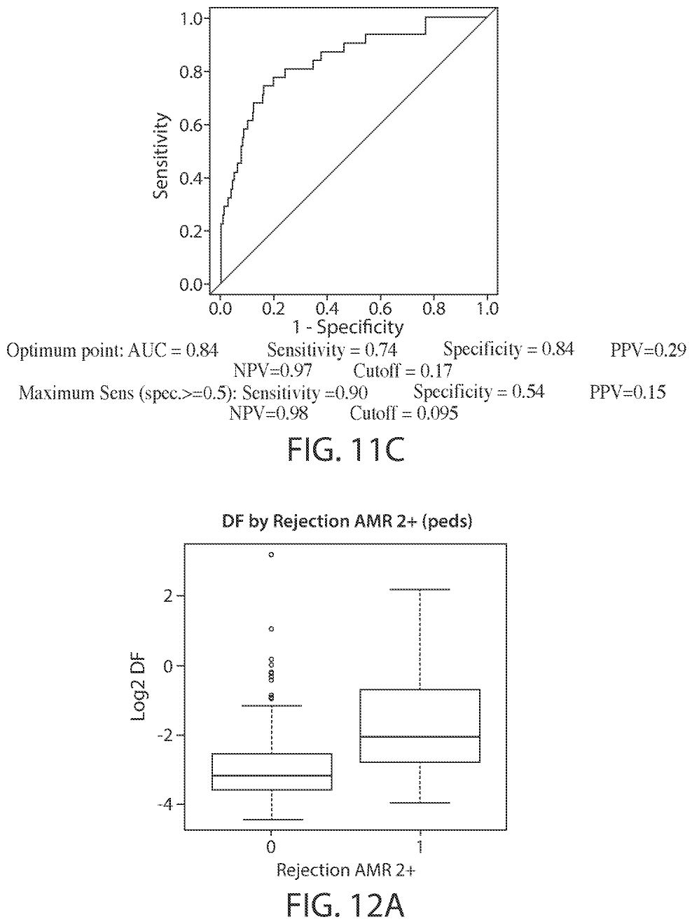

First, rejection (defined as AMR2 or greater) was compared to donor-fraction cell-free DNA (all samples). The results are shown in FIGS. 11A-11C and the table below.

| | | | | | Std | | Lower | Upper | | |
|---|---|---|---|---|---|---|---|---|---|---|
| amr2pB | Obs | Variable | N | Mean | Dev | Median | Quartile | Quartile | Minimum | Maximum |
| 0 | 345 | df | 345 | 0.17 | 0.54 | 0.09 | 0.07 | 0.13 | 0.04 | 9.20 |
| | | log2_df | 345 | −3.26 | 1.00 | −3.44 | −3.91 | −2.99 | −4.71 | 3.20 |
| 1 | 31 | df | 31 | 1.08 | 1.54 | 0.26 | 0.15 | 1.84 | 0.06 | 5.59 |
| | | log2_df | 31 | −1.27 | 2.00 | −1.95 | −2.77 | 0.88 | −3.96 | 2.48 |

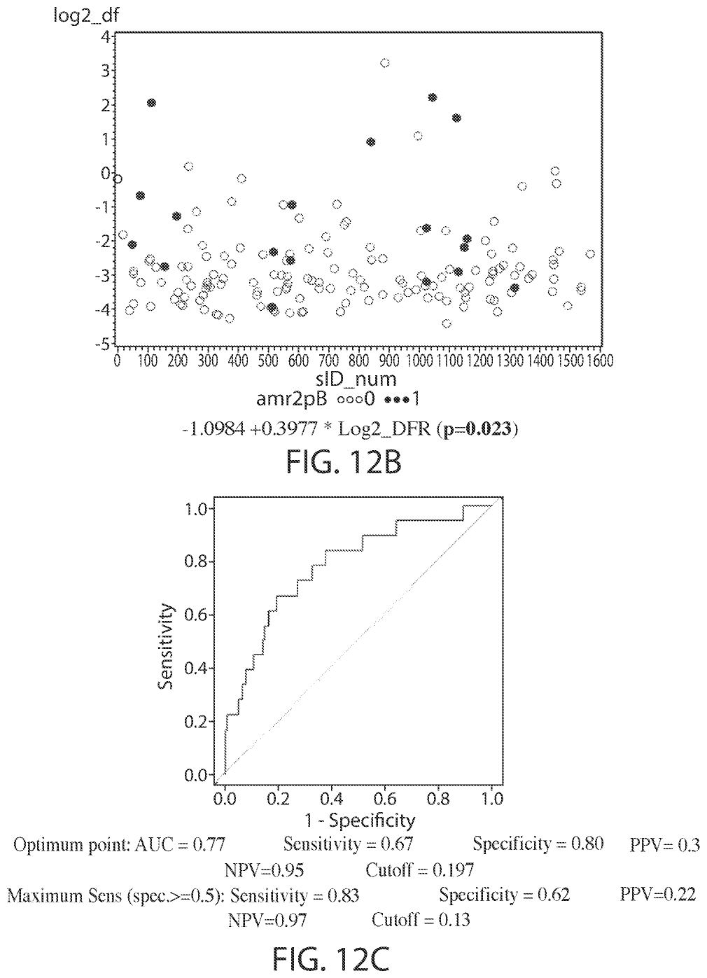

Rejection (defined as AMR2 or greater) was compared to donor-fraction cell-free DNA among pediatric subjects. The results are shown in FIGS. 12A-12C and the table below.

| | N | | | | Std | | Lower | Upper | | |
|---|---|---|---|---|---|---|---|---|---|---|
| amr2pB | Obs | Variable | N | Mean | Dev | Median | Quartile | Quartile | Minimum | Maximum |
| 0 | 143 | df | 143 | 0.25 | 0.79 | 0.11 | 0.08 | 0.17 | 0.05 | 9.20 |
| | | log2_df | 143 | −2.89 | 1.13 | −3.17 | −3.60 | −2.54 | −4.44 | 3.20 |
| 1 | 18 | df | 18 | 0.94 | 1.44 | 0.24 | 0.15 | 0.62 | 0.06 | 4.56 |
| | | log2_df | 18 | −1.41 | 1.90 | −2.04 | −2.77 | −0.69 | −3.96 | 2.19 |

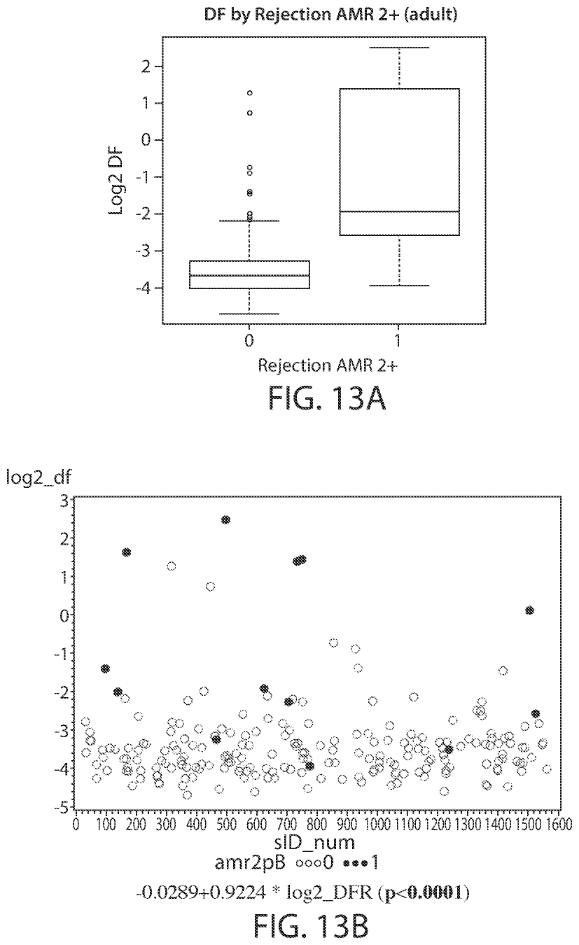

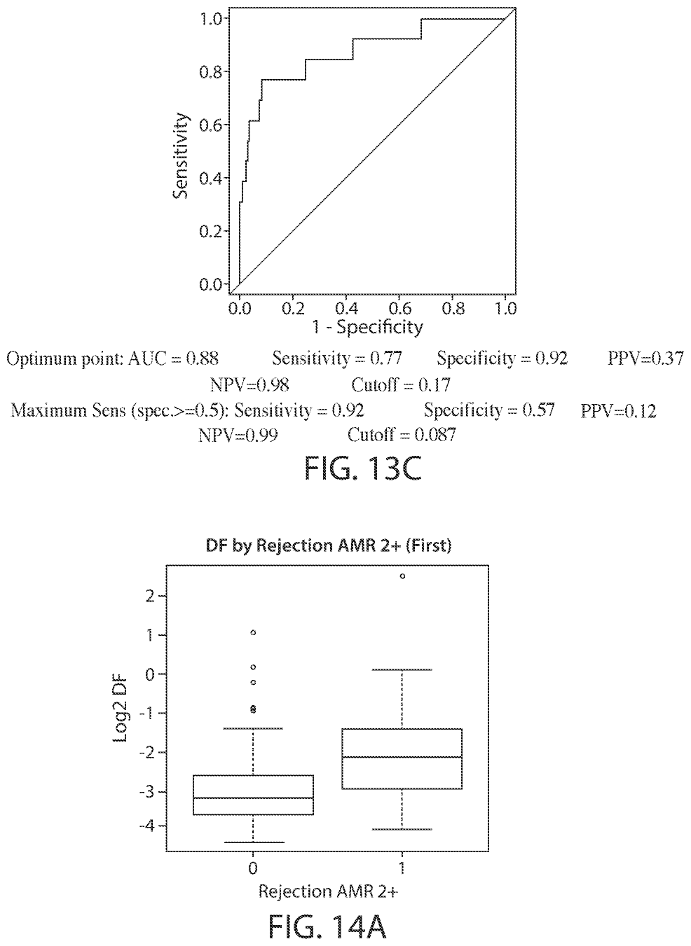

Rejection (defined as AMR2 or greater) was compared to donor-fraction cell-free DNA among adult subjects. The results are shown in FIGS. 13A-13C and the table below.

| | N | | | | Std | | Lower | Upper | | |
|---|---|---|---|---|---|---|---|---|---|---|
| amr2pB | Obs | Variable | N | Mean | Dev | Median | Quartile | Quartile | Minimum | Maximum |
| 0 | 202 | df | 202 | 0.12 | 0.21 | 0.08 | 0.06 | 0.10 | 0.04 | 2.42 |
| | | log2_df | 202 | −3.52 | 0.80 | −3.68 | −4.03 | −3.28 | −4.71 | 1.28 |
| 1 | 13 | df | 13 | 1.28 | 1.72 | 0.26 | 0.17 | 2.61 | 0.06 | 5.59 |
| | | log2_df | 13 | −1.07 | 2.20 | −1.93 | −2.58 | 1.38 | −3.95 | 2.48 |

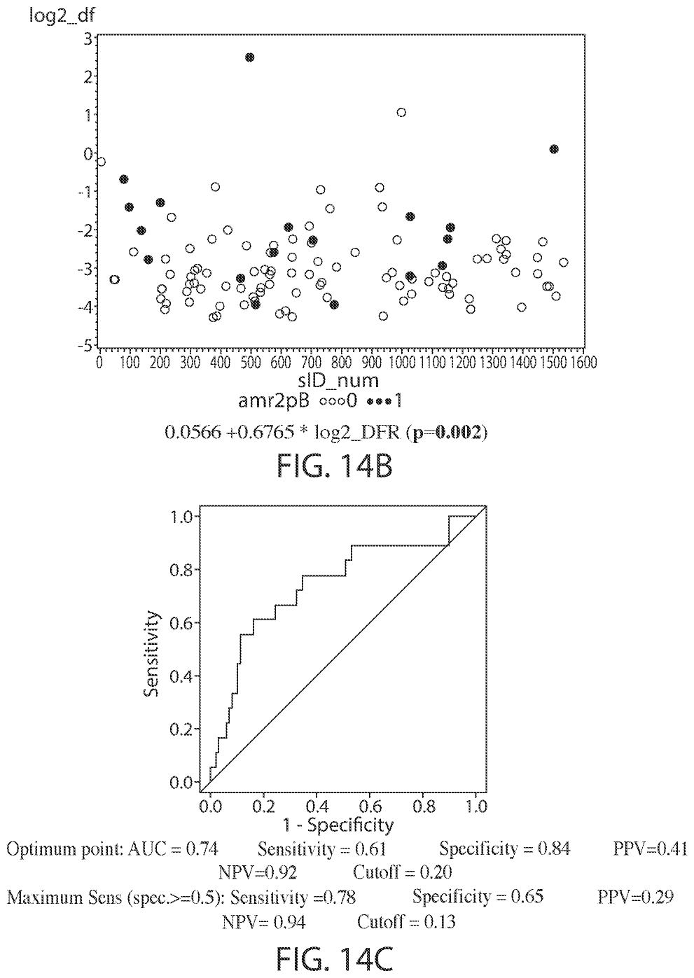

Rejection (defined as AMR2 or greater) was compared to donor-fraction cell-free DNA using the first sample from all subjects. The results are shown in FIGS. 14A-14C and the table below.

| | N | | | | Std | | Lower | Upper | | |
|---|---|---|---|---|---|---|---|---|---|---|
| amr2pB | Obs | Variable | N | Mean | Dev | Median | Quartile | Quartile | Minimum | Maximum |
| 0 | 98 | df | 98 | 0.17 | 0.25 | 0.11 | 0.08 | 0.17 | 0.05 | 2.08 |
| | | log2_df | 98 | −3.01 | 0.96 | −3.18 | −3.60 | −2.59 | −4.29 | 1.05 |
| 1 | 18 | df | 18 | 0.58 | 1.27 | 0.23 | 0.13 | 0.38 | 0.06 | 5.59 |
| | | log2_df | 18 | −1.97 | 1.53 | −2.12 | −2.93 | −1.41 | −3.96 | 2.48 |

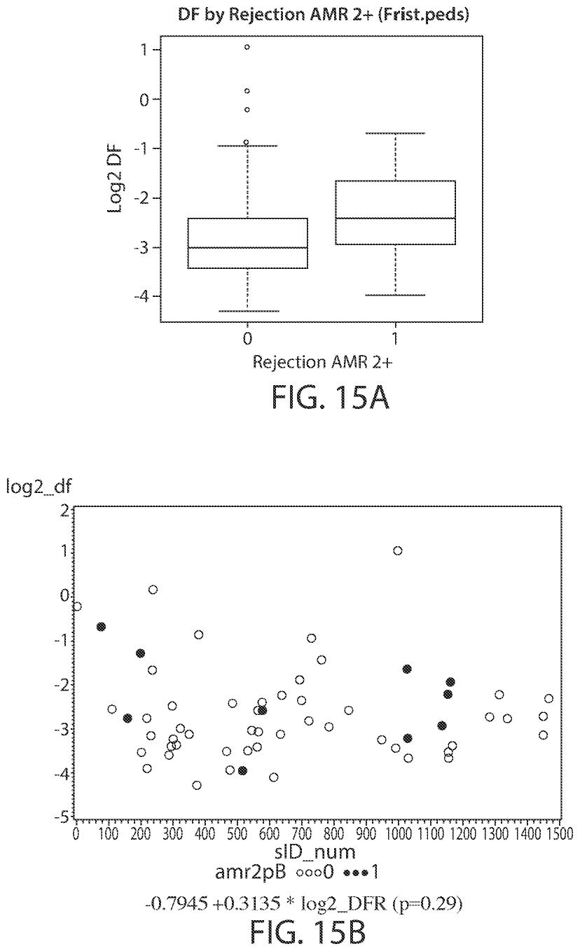

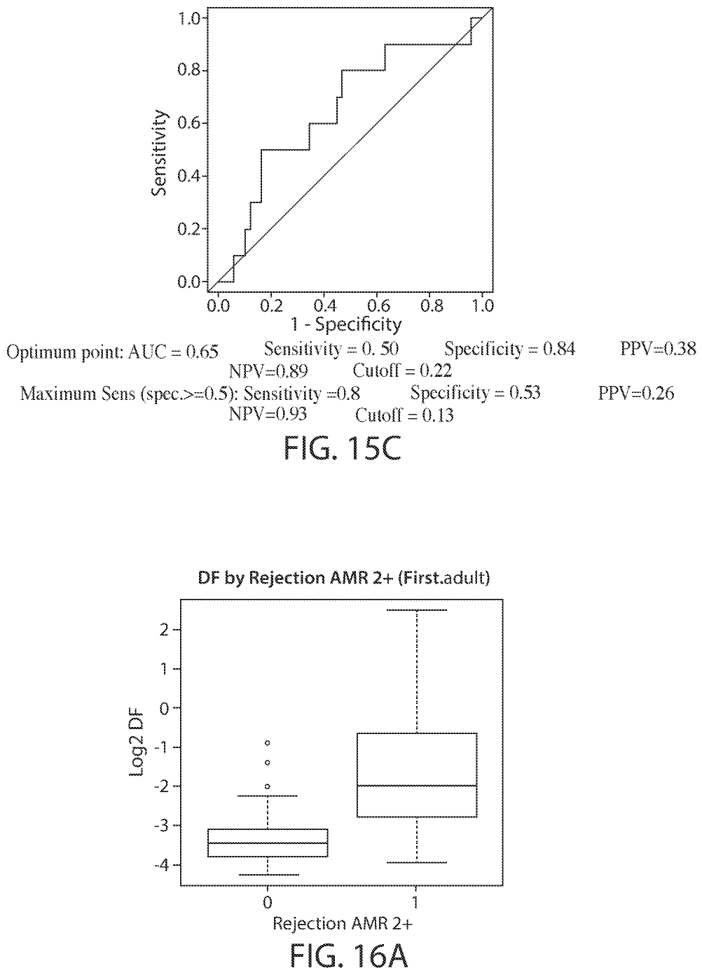

Rejection (defined as AMR2 or greater) was compared to donor-fraction cell-free DNA using the first sample from pediatric subjects. The results are shown in FIGS. 15A-15C and the table below.

| | N | | | | Std | | Lower | Upper | | |
|---|---|---|---|---|---|---|---|---|---|---|
| amr2pB | Obs | Variable | N | Mean | Dev | Median | Quartile | Quartile | Minimum | Maximum |
| 0 | 49 | df | 49 | 0.23 | 0.33 | 0.13 | 0.09 | 0.19 | 0.05 | 2.08 |
| | | log2_df | 49 | −2.72 | 1.08 | −3.00 | −3.42 | −2.40 | −4.29 | 1.05 |
| 1 | 10 | df | 10 | 0.24 | 0.17 | 0.19 | 0.13 | 0.32 | 0.06 | 0.62 |
| | | log2_df | 10 | −2.32 | 0.97 | −2.40 | −2.93 | −1.64 | −3.96 | −0.69 |

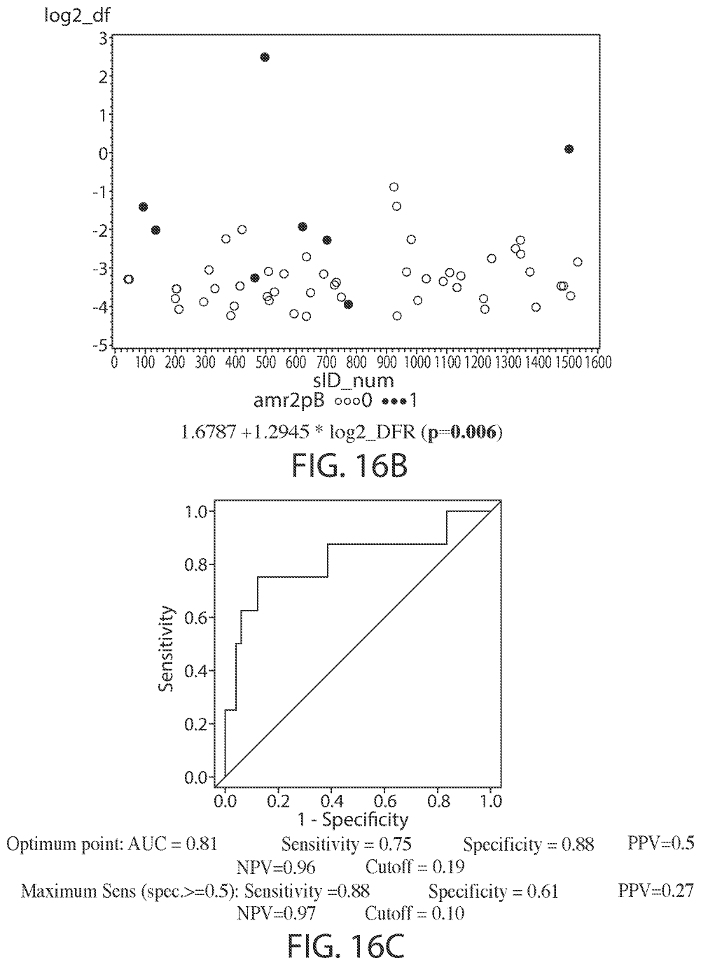

Rejection (defined as AMR2 or greater) was compared to donor-fraction cell-free DNA using the first sample from adult subjects. The results are shown in FIGS. 16A-16C and the table below.

| | N | | | | Std | | Lower | Upper | | |
|---|---|---|---|---|---|---|---|---|---|---|
| amr2pB | Obs | Variable | N | Mean | Dev | Median | Quartile | Quartile | Minimum | Maximum |
| 0 | 49 | df | 49 | 0.12 | 0.09 | 0.09 | 0.07 | 0.12 | 0.05 | 0.54 |
| | | log2_df | 49 | −3.29 | 0.72 | −3.44 | −3.79 | −3.09 | −4.27 | −0.89 |
| 1 | 8 | df | 8 | 0.99 | 1.89 | 0.25 | 0.16 | 0.72 | 0.06 | 5.59 |
| | | log2_df | 8 | −1.53 | 2.02 | −1.98 | −2.77 | −0.66 | −3.95 | 2.48 |

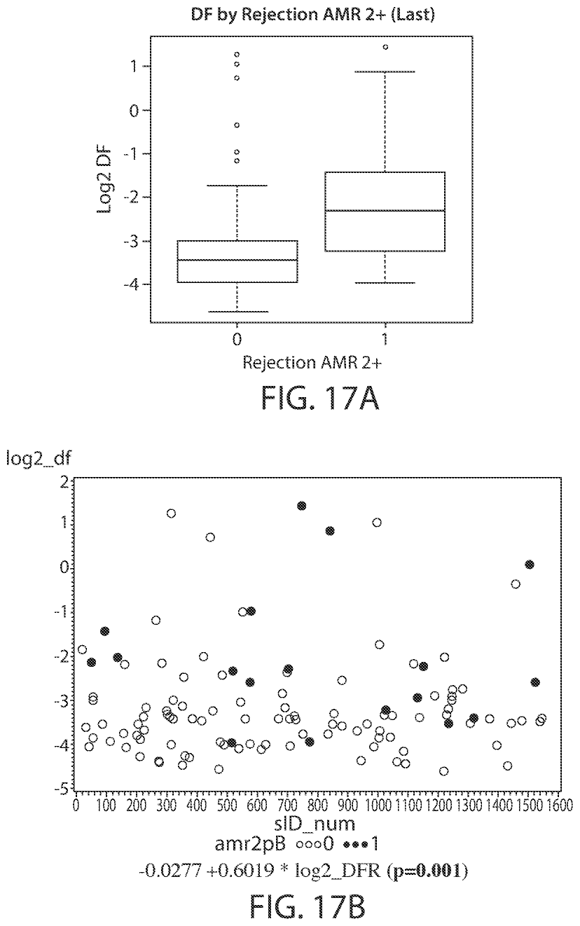

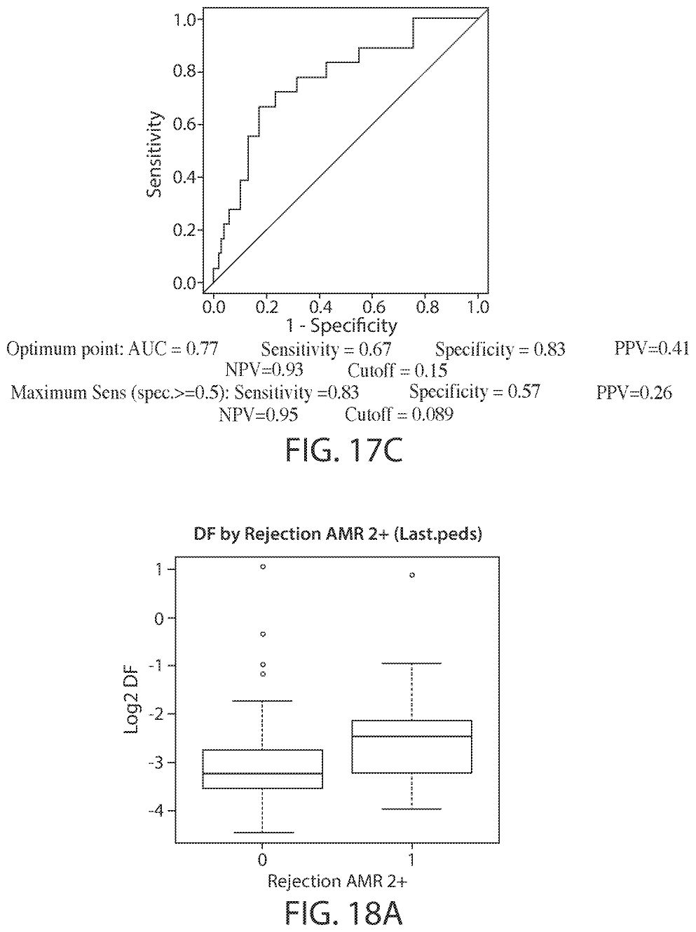

Rejection (defined as AMR2 or greater) was compared to donor-fraction cell-free DNA using the last sample from all subjects. The results are shown in FIGS. 17A-17C and the table below.

| | N | | | | Std | | Lower | Upper | | |
|---|---|---|---|---|---|---|---|---|---|---|
| amr2pB | Obs | Variable | N | Mean | Dev | Median | Quartile | Quartile | Minimum | Maximum |
| 0 | 98 | df | 98 | 0.18 | 0.35 | 0.09 | 0.07 | 0.13 | 0.04 | 2.42 |
| | | log2_df | 98 | −3.25 | 1.10 | −3.43 | −3.94 | −2.99 | −4.60 | 1.28 |
| 1 | 18 | df | 18 | 0.47 | 0.71 | 0.20 | 0.11 | 0.38 | 0.06 | 2.72 |
| | | log2_df | 18 | −2.06 | 1.56 | −2.31 | −3.22 | −1.41 | −3.96 | 1.44 |

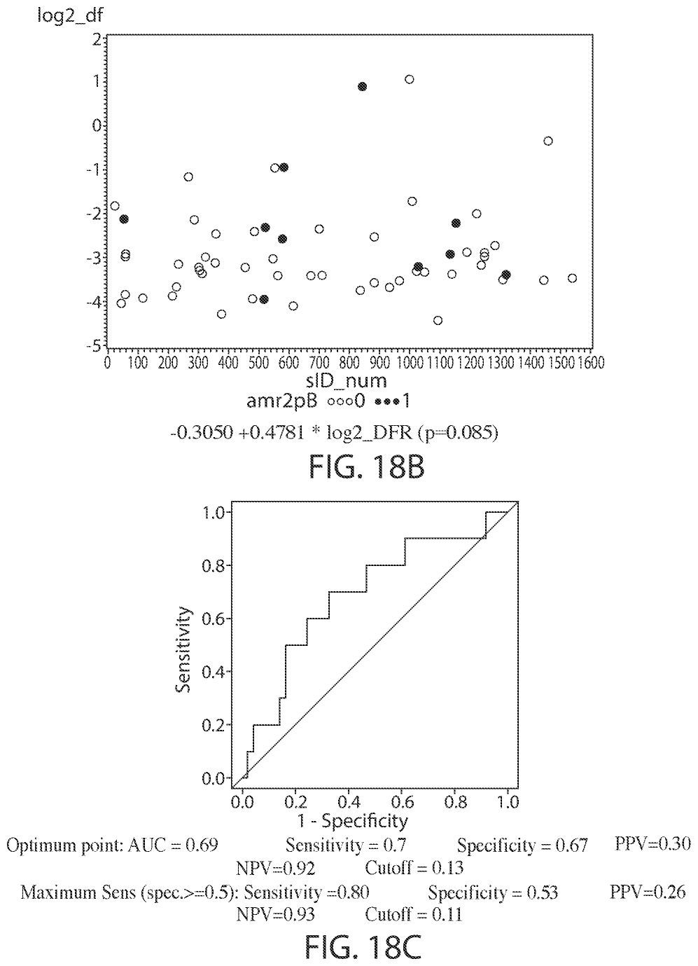

Rejection (defined as AMR2 or greater) was compared to donor-fraction cell-free DNA using the last sample from pediatric subjects. The results are shown in FIGS. 18A-18C and the table below.

| | N | | | | | | Lower | Upper | | |
|---|---|---|---|---|---|---|---|---|---|---|
| amr2pB | Obs | Variable | N | Mean | Std Dev | Median | Quartile | Quartile | Minimum | Maximum |
| 0 | 49 | df | 49 | 0.19 | 0.31 | 0.11 | 0.09 | 0.15 | 0.05 | 2.08 |
| | | log2_df | 49 | −2.99 | 1.03 | −3.23 | −3.53 | −2.73 | −4.44 | 1.05 |
| 1 | 10 | df | 10 | 0.36 | 0.54 | 0.18 | 0.11 | 0.23 | 0.06 | 1.84 |
| | | log2_df | 10 | −2.29 | 1.38 | −2.46 | −3.22 | −2.14 | −3.96 | 0.88 |

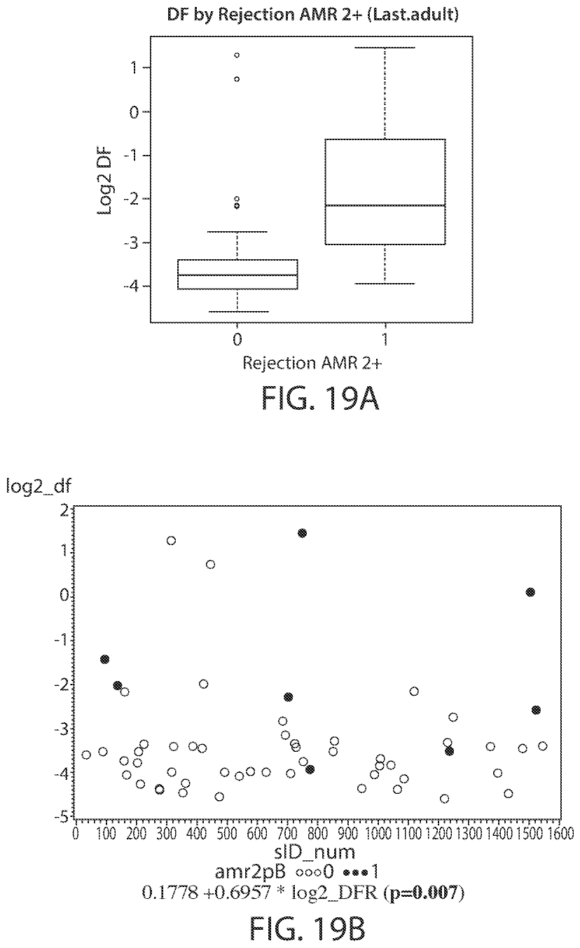

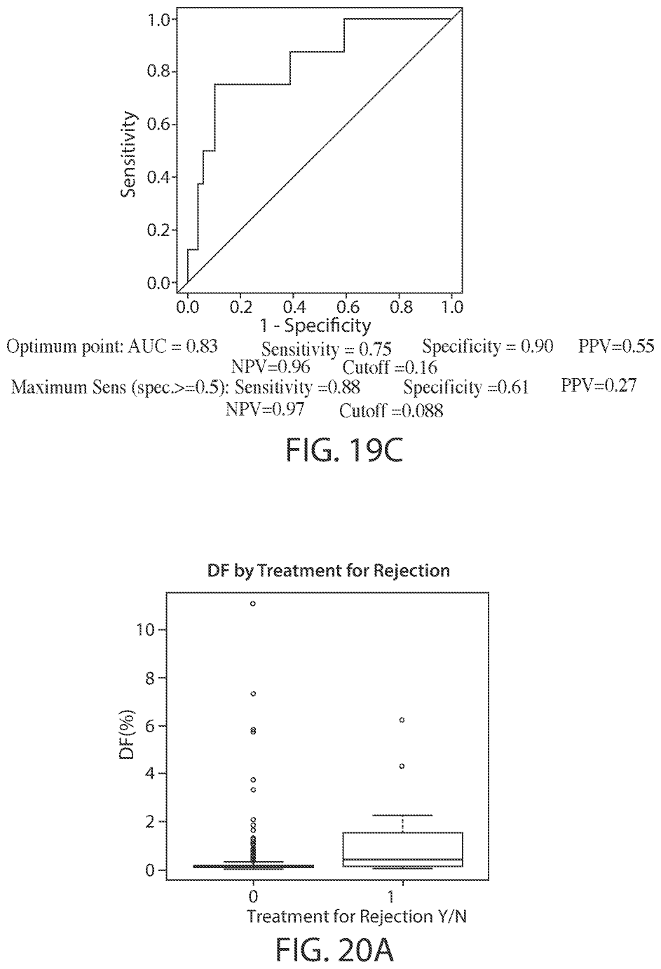

Rejection (defined as AMR2 or greater) was compared to donor-fraction cell-free DNA using the last sample from adult subjects. The results are shown in FIGS. 19A-19C and the table below.

| | N | | | | Std | | Lower | Upper | | |
|---|---|---|---|---|---|---|---|---|---|---|
| amr2pB | Obs | Variable | N | Mean | Dev | Median | Quartile | Quartile | Minimum | Maximum |
| 0 | 49 | df | 49 | 0.16 | 0.40 | 0.07 | 0.06 | 0.09 | 0.04 | 2.42 |
| | | log2_df | 49 | −3.51 | 1.12 | −3.75 | −4.07 | −3.40 | −4.60 | 1.28 |
| 1 | 8 | df | 8 | 0.62 | 0.91 | 0.23 | 0.13 | 0.72 | 0.06 | 2.72 |
| | | log2_df | 8 | −1.78 | 1.80 | −2.15 | −3.05 | −0.66 | −3.95 | 1.44 |

### Example 4—Treatment for Rejection and Donor-Specific (Donor-Fraction) Cell-Free DNA (ALU Ratio Cutoff=0.50)

In this Example, the relationship between outcome (treatment for rejection) and donor-fraction cell-free DNA was examined using an Alu ratio cutoff of 0.50.

The exclusion criteria were as follows:

Exclusion criteria

| | |
|---|---|
| 1. Clinical exclusions | Less than 8 days post-transplant (samples within 7 days |
| | post-transplant) |
| | Pregnant |
| | Have another transplanted organ, including bone marrow |
| | (anyone with such case) |
| | Have post-transplant lymphoproliferative disease (if they |
| | ever had it) - anyone with PTLD |
| | Have cancer or have had cancer in the previous 2 years |
| | (anyone with such case) |
| | Mechanical Circulatory Support at the time of collection |
| | (samples drawn during the procedure) |
| 2. Pre-Genotype Quality | Temperature (Failed for temp- less than 7 C., greater than |
| Control exclusions | 30 C., no monitor, monitor not turned on) |
| | time to spin (Failed for time to spin- more than 5 days) |
| | plasma volume (Failed for plasma volume- method has not |
| | been validated for less than 1 ml) |
| | Failed for DNA yiel |
| 3. Post-Genotype Quality | M2rCV greater than 1.0000 |
| Control exclusions | M2Skew greater than 1.010 |
| | Status of QC Hold |
| | ALU Ratio greater than 0.5 and Samples with N/A for ALU |
| | ALU Ratio greater than 0.35 and Samples with N/A for ALU |
| | ALU Ratio greater than 0.22 and Samples with N/A for ALU |
| Exclusions for healthy controls | Samples within 30 days of death |
| (any one of these criteria) | Samples within 30 days of cardiac arrest (±30 days) |
| | Samples associated to CAV |
| | Samples taken within 14 days prior to initiation of |
| | treatment for infection and 3 weeks after initiation. |
| | Samples during MCS and 1 week after |
| | Samples within 30 days of * time zero (±30 days) |
| | Samples within 30 days of ** positive cardiac biopsy |
| | (±30 days) |

* time zero is onset of treatment for new episode of rejection
** positive cardiac biopsy is defined as CR1+ and or pAMR 1+ (±30 days)
Undecided CR should be excluded
Undecided AMR not excluded unless CR is excluded
Positive but not recorded cases for CR and pAMR were treated as positive cardiac biopsy as well.

Initially, the data from 2537 samples (241 subjects) was subjected to the exclusion criteria noted above, resulting in 1233 samples from 195 subjects. Of those samples, there were 22 treatment for rejection samples (i.e., samples drawn within 24 hours before Time 0 (onset of treatment for rejection)). Out of the 1233 samples, there were 97 plasma samples and 1136 whole blood samples. Three different ALU ratio thresholds were considered: greater than 0.5 (1218 samples from 195 subjects; 22 treatment for rejection samples), greater than 0.35 (1119 samples from 189 subjects; 22 treatment for rejection samples), and greater than 0.22 (463 samples from 130 subjects; 11 treatment for rejection samples). From this analysis, further analysis was performed on the samples that had an ALU ratio equal to or greater than 0.5 (1218 samples from 195 subjects; 22 treatment for rejection samples).

### Statistical Methods

Treatment for rejection was defined as a sample drawn within 24 hours before Time 0 (onset of treatment for a new episode of rejection). If there was more than one treatment within 30 days, then only the first Time 0 was used (first onset of treatment of rejection).

Generalized Estimating Equations for repeated measures were used (subjects as cluster using AR(1) covariance structures as appropriate) with logit link function.

First and last sample analyses were done using a logistic model, as first/last sample implies the first/last sample related to the outcome for the test group and the first/last healthy sample from rest of the subjects during their participation in the study. ROCs were created for both repeated and one sample cases.

An Alu ratio threshold of 0.50 was used for all results described herein.

### Summary of Results

The following table presents the results from the analysis, which are described in more detail below.

| | | p-value | AUC | Sensitivity | Specificity | Cutoff | NPV | PPV |
|---|---|---|---|---|---|---|---|---|
| DFR | All samples | 0.03 | 0.81 | 0.91 | 0.62 | 0.13 | 0.99 | 0.10 |
| | Last sample | 0.008 | 0.82 | 0.90 | 0.67 | 0.13 | 0.98 | 0.30 |
| | First sample | 0.008 | 0.84 | 0.90 | 0.72 | 0.13 | 0.98 | 0.34 |
| log 2 DFR | All samples | <0.0001 | 0.81 | 0.91 | 0.62 | −2.94 | 0.99 | 0.10 |
| | Last sample | <0.0001 | 0.82 | 0.90 | 0.67 | −2.95 | 0.98 | 0.30 |
| | First sample | <0.0001 | 0.84 | 0.90 | 0.72 | 0.13 | 0.98 | 0.34 |

Note that the Sensitivity/Specificity/PPV/NPV/Cutoff reported in the table above are at the optimum point (i.e., largest of sensitivity + specificity). The results showed that DF(%) (and log2DF) was significantly higher for Treatment for Rejection samples.

### Results

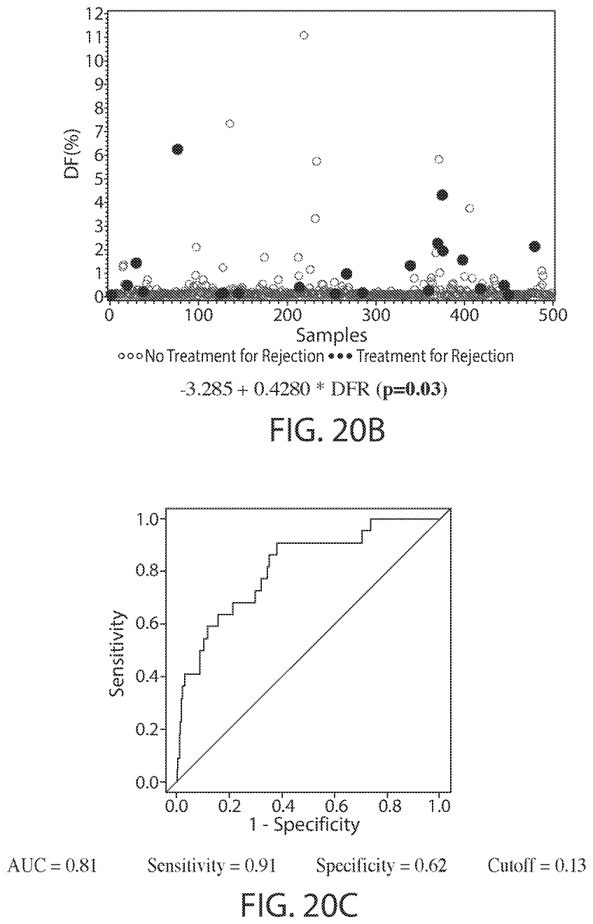

First, donor-fraction (donor-specific) cell-free DNA from the treatment for rejection (“1”) and healthy (“0”) groups was compared. The results are shown in FIGS. 20A-20C and the table below.

Analysis Variable : DF

| Trt | | | Std | | Lower | Upper | | |
|---|---|---|---|---|---|---|---|---|
| Rejection | N | Mean | Dev | Median | Quartile | Quartile | Minimum | Maximum |
| 0 | 477 | 0.26 | 0.77 | 0.11 | 0.08 | 0.18 | 0.04 | 11.11 |
| 1 | 22 | 1.15 | 1.55 | 0.43 | 0.15 | 1.56 | 0.08 | 6.26 |

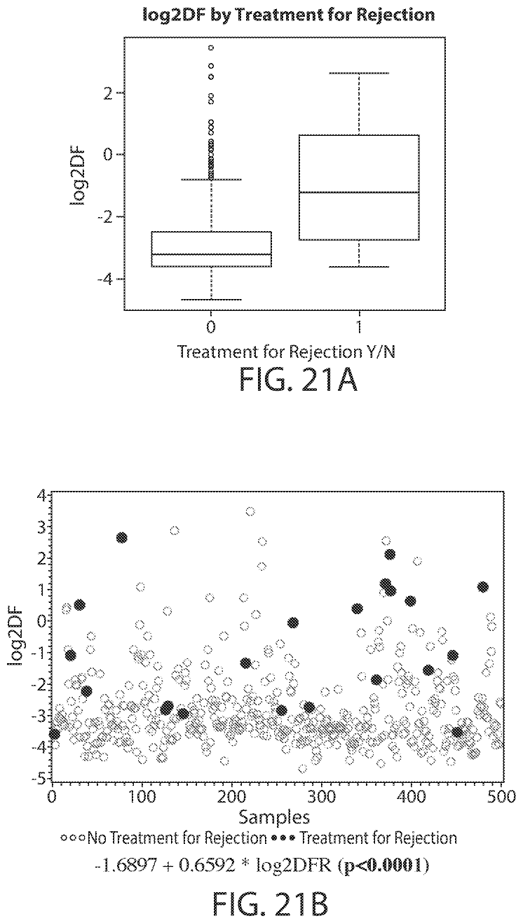

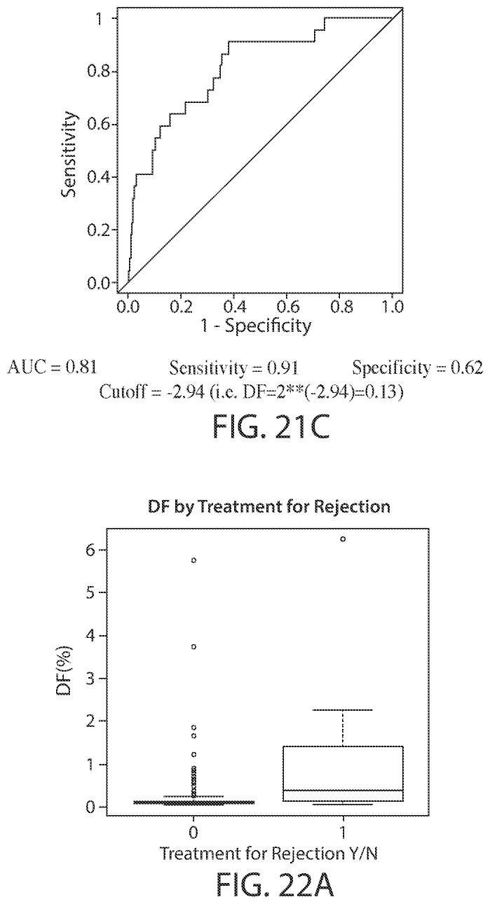

The results were then analyzed using log 2 of the donor-fraction (donor-specific) cell-free DNA. The results are shown in FIGS. 21A-21C and the table below.

Analysis Variable: log2_df

| Trt | | | Std | | Lower | Upper | | |
|---|---|---|---|---|---|---|---|---|
| Rejection | N | Mean | Dev | Median | Quartile | Quartile | Minimum | Maximum |
| 0 | 477 | −2.85 | 1.21 | −3.21 | −3.60 | −2.47 | −4.69 | 3.47 |
| 1 | 22 | −0.95 | 1.90 | −1.21 | −2.74 | 0.64 | −3.59 | 2.65 |

The negative and positive predictive values (NPV and PPV, respectively) were examined for both the donor-fraction cell-free DNA, and the log 2 of the donor-fraction cell-free DNA. The results were identical:

(M2=0.13)=(log2DF=−2.94)

TABLE of group by trtrej

| | trtrej | | |
|---|---|---|---|
| group | trtrej | Healthy | Total |
| M2 > 0.13 | 20 | 183 | 203 |
| M2 <= 0.13 | 2 | 294 | 296 |
| Total | 22 | 477 | 499 |

PPV=20/(20+183)=0.10
NPV=294/(2+294)=0.99
Sensitivity=20/(20+2)=0.91
Specificity=294/(183+294)=0.62

TABLE of group by trtrej

| group | Trtrej | | |
|---|---|---|---|
| Frequency | trtrej | Healthy | Total |
| log2DF > −2.94 | 20 | 183 | 203 |
| log2DF <= −2.94 | 2 | 294 | 296 |
| Total | 22 | 477 | 499 |

PPV=20/(20+183)=0.10
NPV=294/(2+294)=0.99
Sensitivity=20/(20+2)=0.91
Specificity=294/(183+294)=0.62

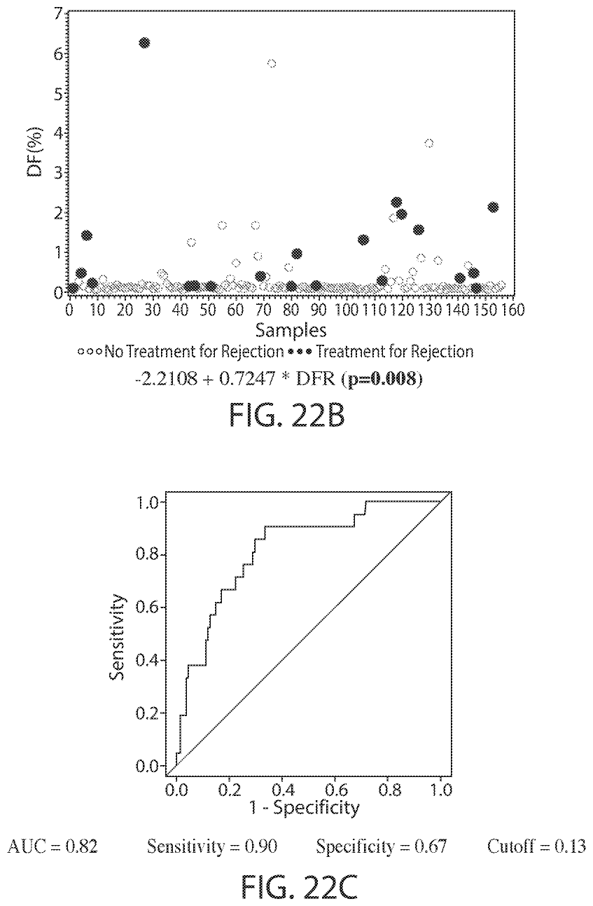

Donor-fraction (donor-specific) cell-free DNA from the treatment for rejection (“1”) and healthy (“0”) groups using the last samples collected was compared. The results are shown in FIGS. 22A-22C and the table below.

Analysis Variable: DF (%)

| Trt | | | Std | | Lower | Upper | | |
|---|---|---|---|---|---|---|---|---|
| Rejection | N | Mean | Dev | Median | Quartile | Quartile | Minimum | Maximum |
| 0 | 135 | 0.26 | 0.64 | 0.10 | 0.08 | 0.15 | 0.05 | 5.75 |
| 1 | 21 | 1.00 | 1.41 | 0.40 | 0.15 | 1.42 | 0.08 | 6.26 |

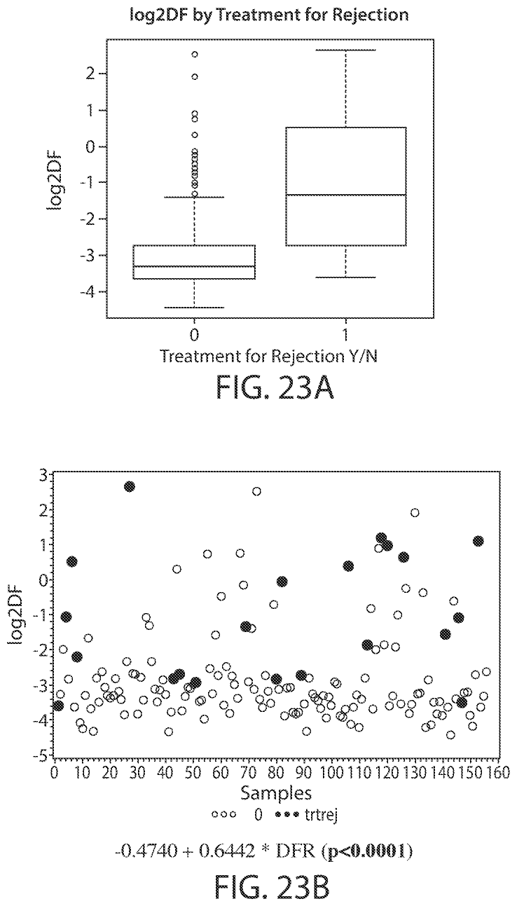

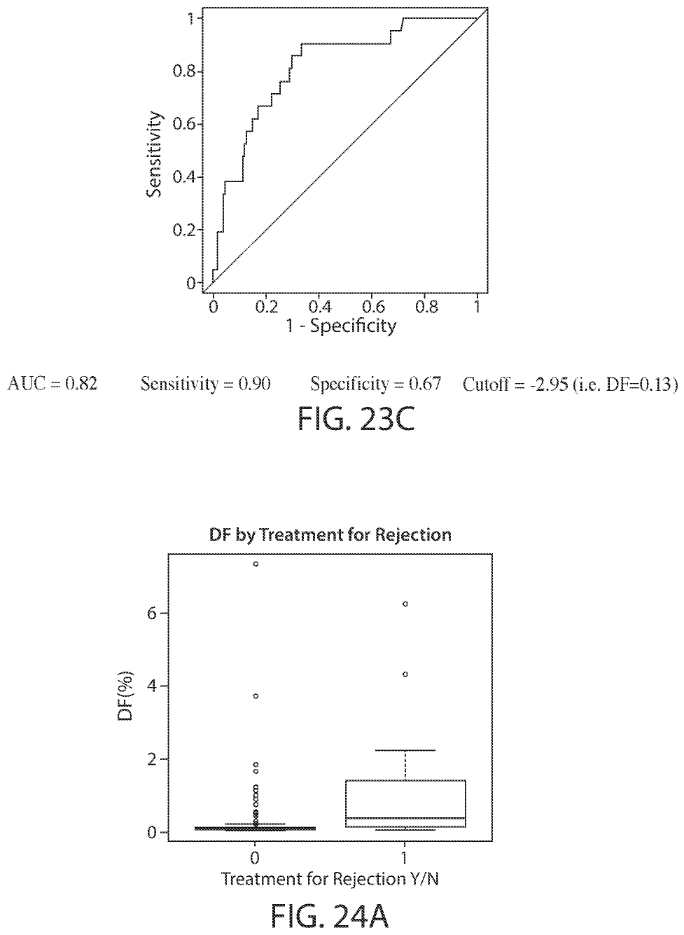

The same analysis was repeated using log 2 donor-fraction (donor-specific) cell-free DNA from the treatment for rejection (“1”) and healthy (“0”) groups using the last samples collected was compared. The results are shown in FIGS. 23A-23C and the table below.

Analysis Variable: log2_df

| Trt | | | Std | | Lower | Upper | | |
|---|---|---|---|---|---|---|---|---|
| Rejection | N | Mean | Dev | Median | Quartile | Quartile | Minimum | Maximum |
| 0 | 135 | −2.90 | 1.27 | −3.30 | −3.64 | −2.73 | −4.42 | 2.52 |
| 1 | 21 | −1.09 | 1.81 | −1.34 | −2.74 | 0.51 | −3.59 | 2.65 |

The negative and positive predictive values (NPV and PPV, respectively) were examined for both the donor-fraction cell-free DNA and the log 2 of the donor-fraction cell-free DNA (last sample). The results were identical:

(M2=0.13)=(log2DF=−2.95)

Table of group by trtrej

| group | Trtrej | | |
|---|---|---|---|
| Frequency | trtrej | Healthy | Total |
| M2 > 0.13 | 19 | 45 | 64 |
| M2 <= 0.13 | 2 | 90 | 92 |
| Total | 21 | 135 | 156 |

PPV=19/(19+45)=0.30
NPV=90/(2+90)=0.98
Sensitivity=19/(19+2)=0.90
Specificity=90/(45+90)=0.67

TABLE of group by trtrej

| group | Trtrej | | |
|---|---|---|---|
| Frequency | trtrej | Healthy | Total |
| log2DF > −2.95 | 19 | 45 | 64 |
| log2DF <= −2.95 | 2 | 90 | 92 |
| Total | 21 | 135 | 156 |

PPV=19/(19+45)=0.30
NPV=90/(2+90)=0.98
Sensitivity=19/(19+2)=0.90
Specificity=90/(45+90)=0.67

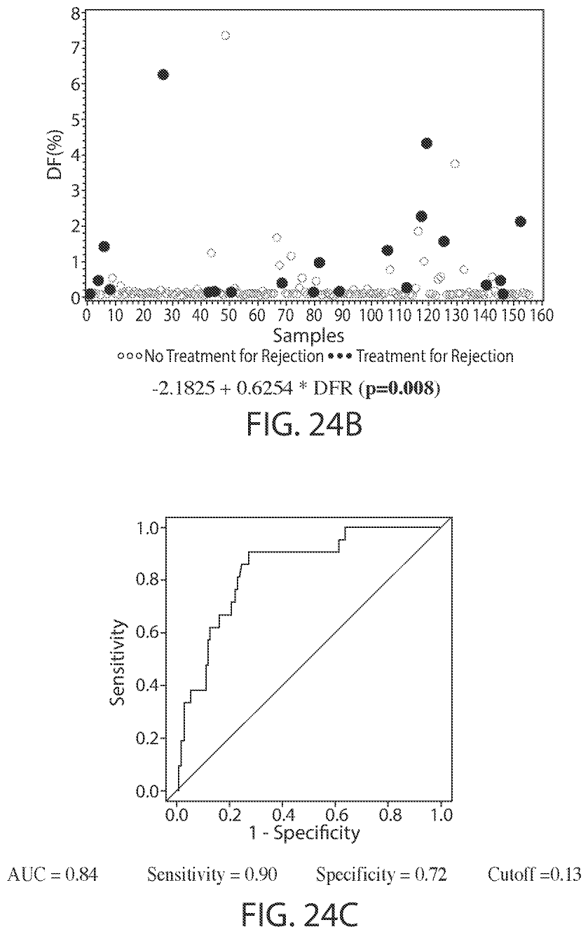

Donor-fraction (donor-specific) cell-free DNA from the treatment for rejection (“1”) and healthy (“0”) groups using the first samples collected was compared. The results are shown in FIGS. 24A-24C and the table below.

Analysis Variable: DF (%)

| Trt | | | Std | | Lower | Upper | | |
|---|---|---|---|---|---|---|---|---|
| Rejection | N | Mean | Dev | Median | Quartile | Quartile | Minimum | Maximum |
| 0 | 135 | 0.26 | 0.74 | 0.10 | 0.07 | 0.14 | 0.04 | 7.35 |
| 1 | 21 | 1.11 | 1.58 | 0.40 | 0.15 | 1.42 | 0.08 | 6.26 |

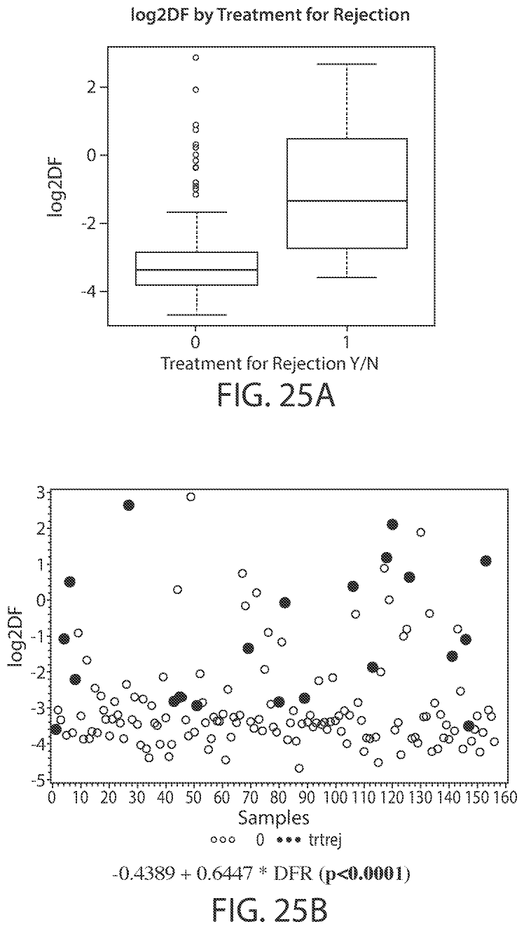

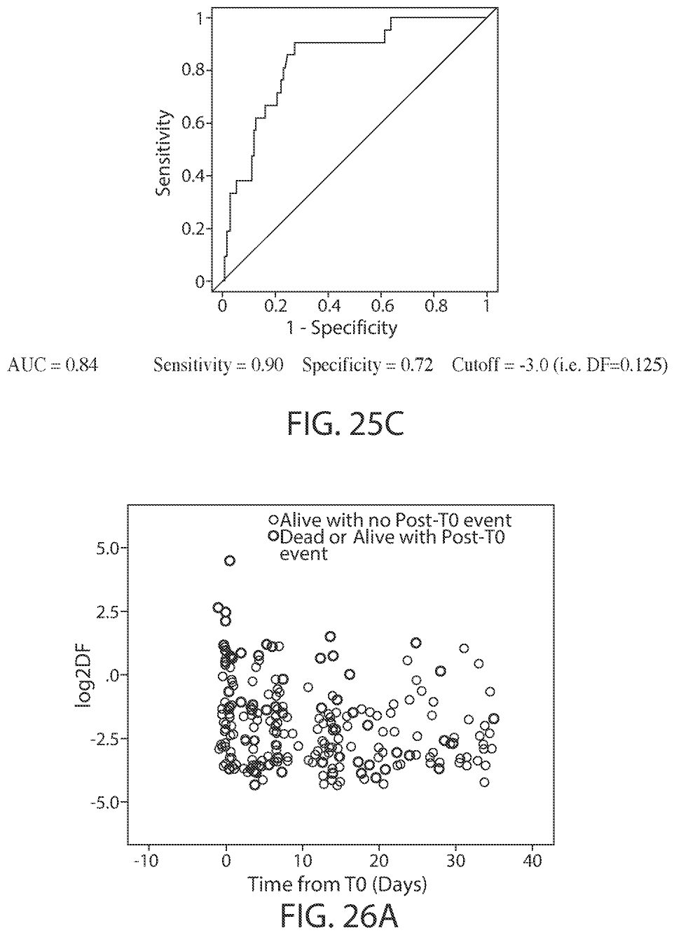

The same analysis was repeated using log 2 donor-fraction (donor-specific) cell-free DNA from the treatment for rejection (“1”) and healthy (“0”) groups using the first samples collected was compared. The results are shown in FIGS. 25A-25C and the table below.

Analysis Variable: log2_df

| | | | Std | | Lower | Upper | | |
|---|---|---|---|---|---|---|---|---|
| trtrej | N | Mean | Dev | Median | Quartile | Quartile | Minimum | Maximum |
| 0 | 135 | −3.01 | 1.30 | −3.37 | −3.81 | −2.83 | −4.69 | 2.88 |
| 1 | 21 | −1.04 | 1.89 | −1.34 | −2.74 | 0.51 | −3.59 | 2.65 |

The negative and positive predictive values (NPV and PPV, respectively) were examined for both the donor-fraction cell-free DNA and the log 2 of the donor-fraction cell-free DNA (last sample). The results were identical:

(M2=0.13)==(log2DF=−2.95)
(M2=0.125)=(log2DF=−3)

TABLE of group by trtrej

| group | trtrej | | |
|---|---|---|---|
| Frequency | trtrej | Healthy | Total |
| M2 > 0.13 | 19 | 37 | 56 |
| M2 <= 0.13 | 2 | 98 | 100 |
| Total | 21 | 135 | 156 |

PPV=19/(19+37)=−0.34
NPV=98/(2+98)=0.98
Sensitivity=19/(19+2)=0.90
Specificity=98/(37+98)=0.72

TABLE of group by trtrej

| group | trtrej | | |
|---|---|---|---|
| Frequency | trtrej | Healthy | Total |
| log2DF > −3 | 19 | 37 | 56 |
| log2DF <= −3 | 2 | 98 | 100 |
| Total | 21 | 135 | 156 |

PPV=19/(19+37)=0.34
NPV=98/(2+98)=0.98
Sensitivity=19/(19+2)=0.90
Specificity=98/(37+98)=0.72

### Example 5—Treatment for Rejection and Donor-Specific (Donor-Fraction) Cell-Free DNA Over Time (ALU Ratio Cutoff=0.50)

In this Example, donor-fraction (donor-specific) cell-free DNA percentages/levels were compared over time and with respect to outcome (treatment for rejection). As described in more detail below, it was found that:
When all 234 samples were used Days from T0 was significant and showed a positive relation with log2DF (p<0.0001)
When we limit the samples drawn within 10 days of T0, subjects with post-Event had significantly (p=0.01) higher log2DF; Days from T0 was marginally (p=0.07) significant
Within the time windows: Event group was significantly higher at Day 0 and Day 14
Models with time windows for Post-T0 Event group: log2DF for Day 4, 7 and 28 were significantly lower than Day 0 (p<0.05)
Models with time windows for No Post-T0 Event group: log2DF for Day 14 was significantly lower than Day 0 (p<0.05)
   The exclusion criteria used was as follows:

Exclusion criteria

| | |
|---|---|
| 1. Clinical exclusions | Less than 8 days post-transplant (samples within 7 days |
| | post-transplant) |
| | Pregnant |
| | Have another transplanted organ, including bone marrow |
| | (anyone with such case) |
| | Have post-transplant lymphoproliferative disease (if they |
| | ever had it)-anyone with PTLD |
| | Have cancer or have had cancer in the previous 2 years |
| | (anyone with such case) |
| | Mechanical Circulatory Support at the time of collection |
| | (samples drawn during the procedure) |
| 2. Pre-Genotype Quality | Temperature (Failed for temp-less than 7 C., greater than |
| Control exclusions | 30 C., no monitor, monitor not turned on) |
| | time to spin (Failed for time to spin-more than 5 days) |
| | plasma volume (Failed for plasma volume-method has not |
| | been validated for less than 1 ml) |
| | Failed for DNA yield |
| 3. Post-Genotype Quality | M2rCV greater than 1.0000 |
| Control exclusions | M2Skew greater than 1.010 |
| | Status of QC Hold |
| | ALU Ratio greater than 0.5 and Samples with N/A for |
| | ALU |
| | ALU Ratio greater than 0.35 and Samples with N/A for |
| | ALU |
| | ALU Ratio greater than 0.22 and Samples with N/A for |
| | ALU |
| Exclusions for healthy | Samples within 30days of death |
| controls (any one of these | Samples within 30days of cardiac arrest (±30 days) |
| criteria may be excluded) | Samples associated to CAV |
| | Samples taken within 14days prior to initiation of |
| | treatment for infection and 3 weeks after initiation. |
| | Samples during MCS and 1 week after |
| | Samples within 30 days of * time zero (±30 days) |
| | Samples within 30 days of ** positive cardiac |
| | biopsy (±30 days) |

* time zero is onset of treatment for new episode of rejection
** positive cardiac biopsy is defined as CR1± and or pAMR 1± (±30 days)
Undecided CR should be excluded
Undecided AMR not excluded unless CR is excluded
Positive but not recorded cases for CR and pAMR were treated as positive cardiac biopsy as well.

Initially, the data from 2537 samples (241 subjects) was subjected to the exclusion criteria noted above, resulting in 1233 samples from 195 subjects. Of those samples, there were 22 treatment for rejection samples (i.e., samples drawn within 24 hours before Time 0 (onset of treatment for rejection)). Out of the 1233 samples, there were 97 plasma samples and 1136 whole blood samples. Three different ALU ratio thresholds were considered: greater than 0.5 (1218 samples from 195 subjects; 22 treatment for rejection samples), greater than 0.35 (1119 samples from 189 subjects; 22 treatment for rejection samples), and greater than 0.22 (463 samples from 130 subjects; 11 treatment for rejection samples). From this analysis, further analysis was performed on the samples that had an ALU ratio equal to or greater than 0.5 (1218 samples from 195 subjects; 22 treatment for rejection samples).

### Statistical Methods

If any subject had needed mechanical circulatory support (MCS) and/or experienced cardiac arrest (CA) before T0 (onset of transplant complications), then the subject was grouped with the No Post-T0 event group. Eleven of the alive subjects had MCS and/or CA, and of the 11, nine experienced the MCS and/or CA before T0.

For the models, log2DF was used as the outcome, and days from T0 (or the time windows from T0) and post-T0 event (yes/no) were used as covariates.

As there were a number of negative for the log (2)-transformed donor fraction (cell-free DNA) and a Gamma distribution was assumed, all values were converted into positive entries by adding a positive number (+5) in order to include all samples for the model. GEE (Generalized Estimating Equation) with Maximum Likelihood Estimation, appropriate covariance structures for the subject clusters, and identity link function was used for the model. The covariance structures used to fit the best model (best fit) are described below.

Log2DF was compared across/within the consecutive time windows using the repeated measures as well. P-values were adjusted using FDR (false discovery rate).

An Alu ratio threshold of 0.50 was used for all results described herein.

### Results

First, the Time 0 value for each subject was determined. Of the 1218 samples from 195 subjects, 96 of the subjects were treated for rejection and were included in the analysis (734 total samples). These 96 subjects had total 150 episodes of treatment for rejection, but some of these episodes were within 30 days. In total, 58 of the 96 subjects had only one treatment for rejection (i.e., one Time 0), 30 of the 96 subjects had more than one treatment with the time between treatments being greater than 30 days, meaning all treatments for rejection for these subjects were taken as Time 0 (20 subjects had two treatments, seven subjects had three treatments, and three subjects had four treatments), and eight of the 96 subjects had more than one treatment with the time between treatments being 30 days or less (six subjects had two treatments within 30 days. Time 0 was taken as the first treatment; one subject had three treatments and first two were within 30 days, so Time 0 was taken as the first and third treatments; one subject had four treatments, two of which were within 30 days, so Time 0 was taken as the first, third, and fourth treatments). If samples as drawn within 24 hours before Time 0 to 25 days after Time 0, there are 248 samples from 66 subjects. If subjects with at least two samples for the treatment for rejection series are taken, then there are 234 samples from 52 subjects (14 subjects only had one sample).

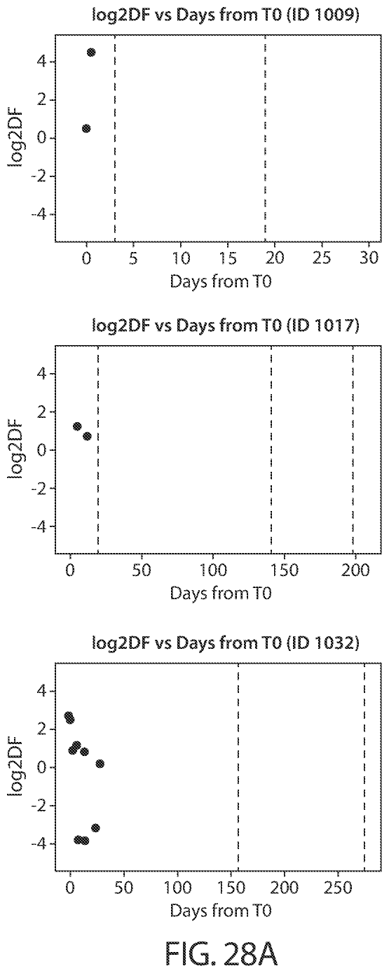

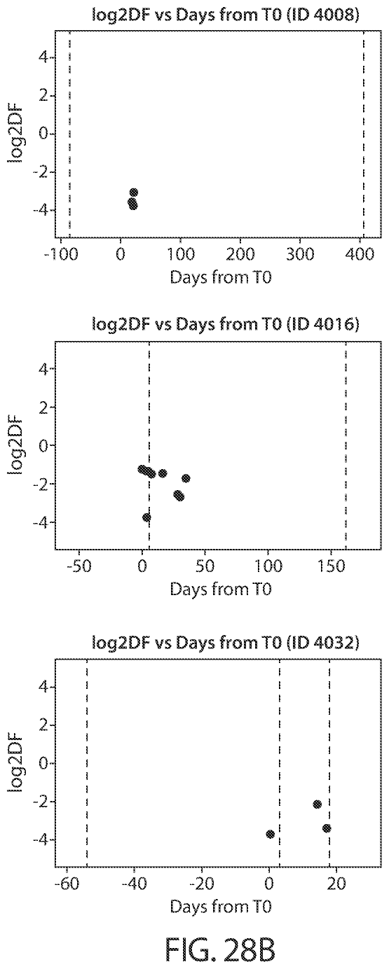

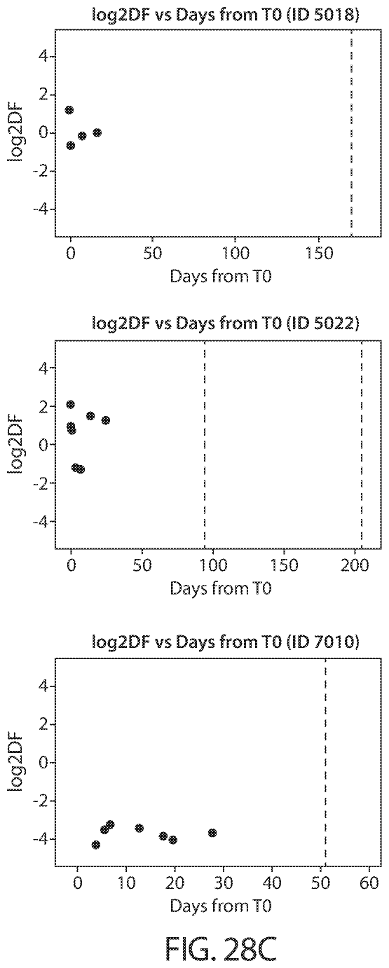

Nine of the 52 subjects died (46 total samples from the subjects). Details are provided in the table below, and the plots for the subjects who died are shown in FIGS. 28A-28C.

| subject_ | # | | Cardiac | Mechanical Circulatory Support |
|---|---|---|---|---|
| id | samples | Death* | Arrest | (MCS) |
| 1009 | 2 | 19 d | | 3 d from T0 |
| 1017 | 2 | 198 d | | 20 d = m1& 141 d = m2 from T0 |
| 1032 | 9 | 275 d | | 157 d and 0 d from T0(1) and |
| | | | | T0(2) respectively** |
| 4008 | 3 | 406 d | | 86 d before T0 |
| 4016 | 9 | 161 d | | 5 d and −53 d from T0(1) and |
| | | | | T0(2) respectively** |
| 4032 | 3 | 18 d | | −54 d = m1 and 3 d = m2 from |
| 5018 | 4 | 170 d | | T0(1) |
| 5022 | 7 | 205 d | 94 d | |
| | | | from | |
| | | | T0(1) | |
| 7010 | 7 | 51 d | | |

*Days between T0 and death calculated using T0(1)
**Using T0(1) to calculate days for the plot since only one MCS

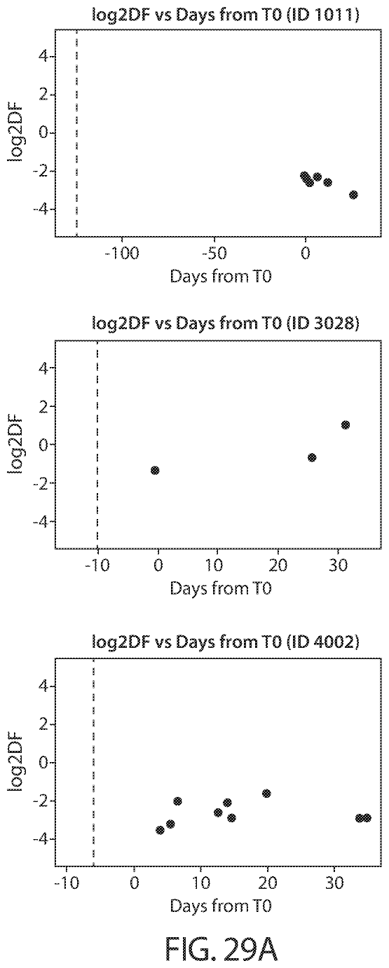

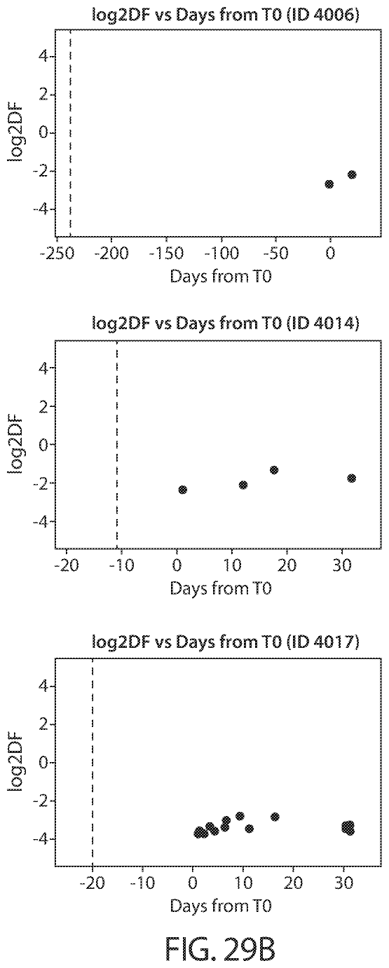

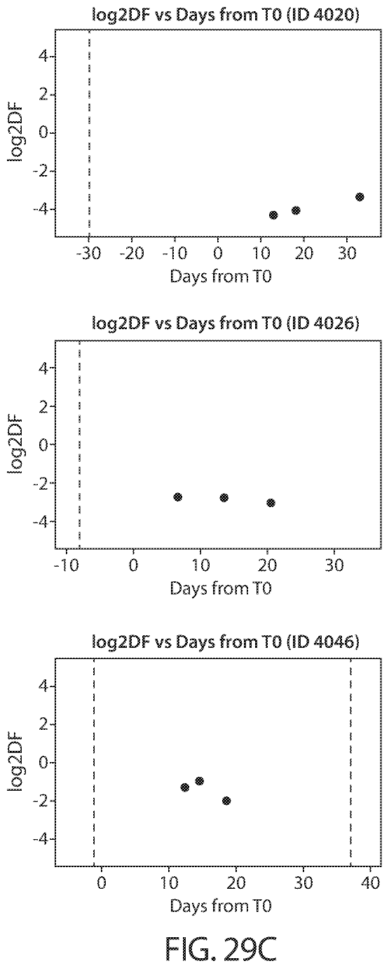

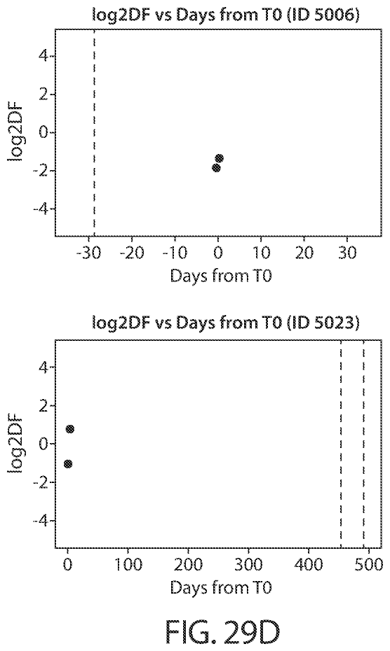

Eleven of the 52 subjects were alive and experienced cardiac arrest (CA) and/or required MCS (51 total samples from the subjects). Nine of the 11 subjects had events (CA and/or MCS) before T0, and only two subjects had post-T0 events. Details are provided in the table below, and the plots for the individual subjects are shown in FIGS. 29A-29D.

| subject_id | # samples | Cardiac Arrest | MCS |
|---|---|---|---|
| 1011 | 6 | | 125 d before T0 |
| 3028 | 3 | 10 d before T0* | 10 d before T0* |
| 4002 | 9 | | 6 d before T0 |
| 4006 | 2 | | 237 d before T0 |
| 4014 | 4 | | 11 d before T0* |
| 4017 | 14 | | 20 d before T0* |
| 4020 | 3 | | 30 d before T0 |
| 4026 | 3 | | 8 d before T0 |
| 4046 | 3 | 1 d before T0 | 1 d before and |
| 5006 | 2 | 29 d before T0* | 37d after T0 |
| 5023 | 2 | 454 d and 491 d after T0 | |

*Days between T0 and CA, MCS calculated using T0(1)

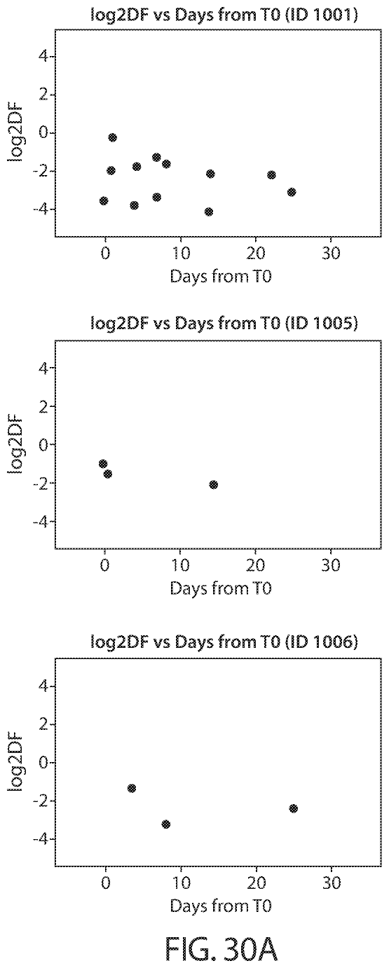

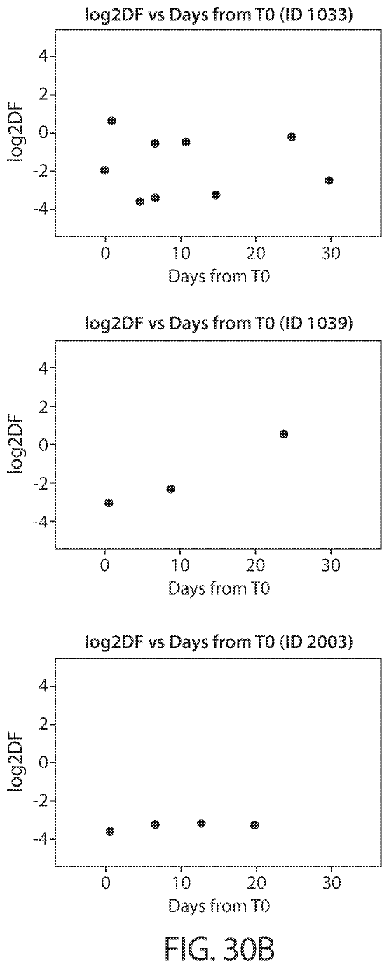

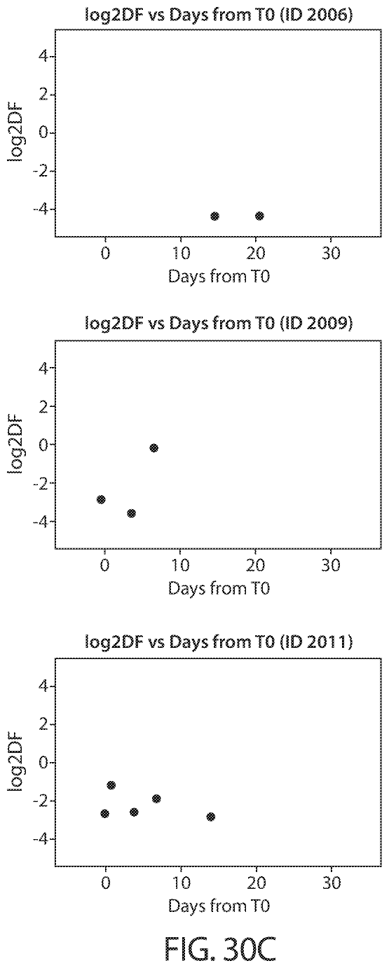

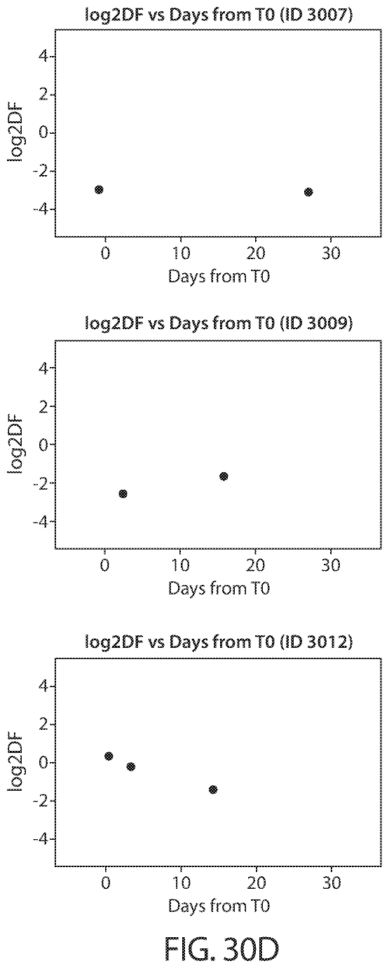

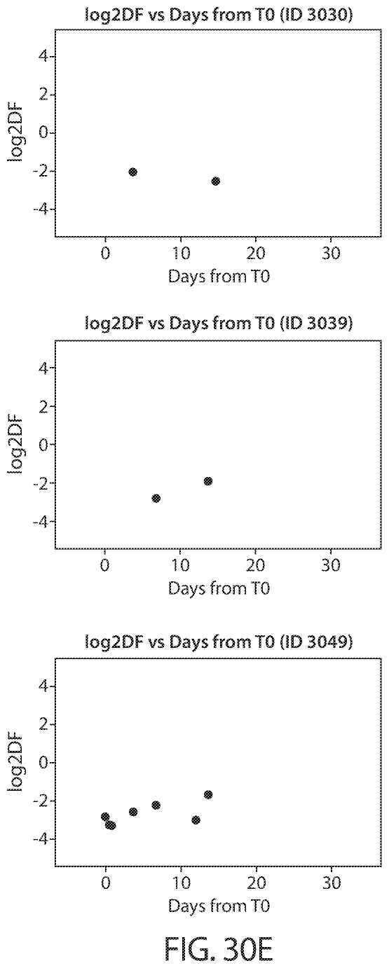

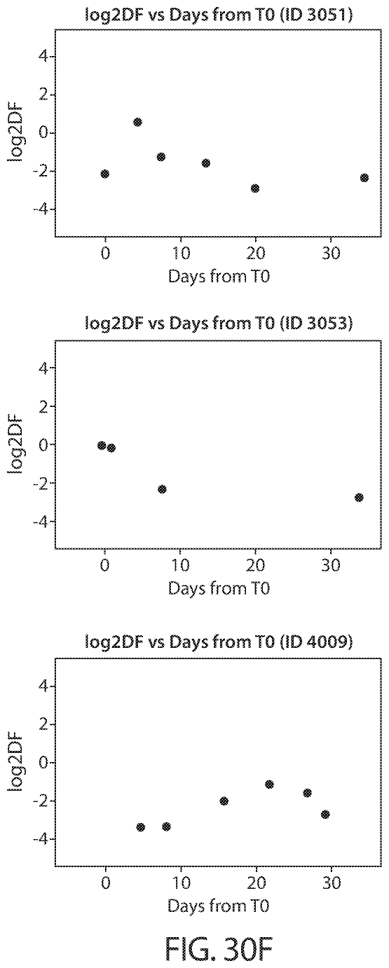

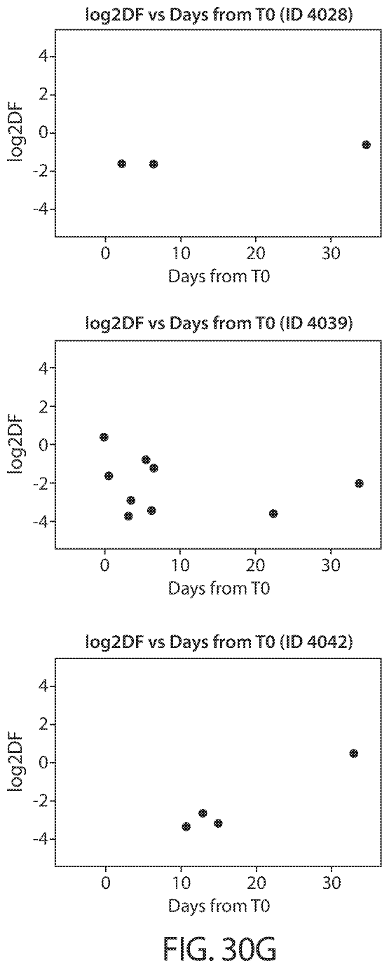

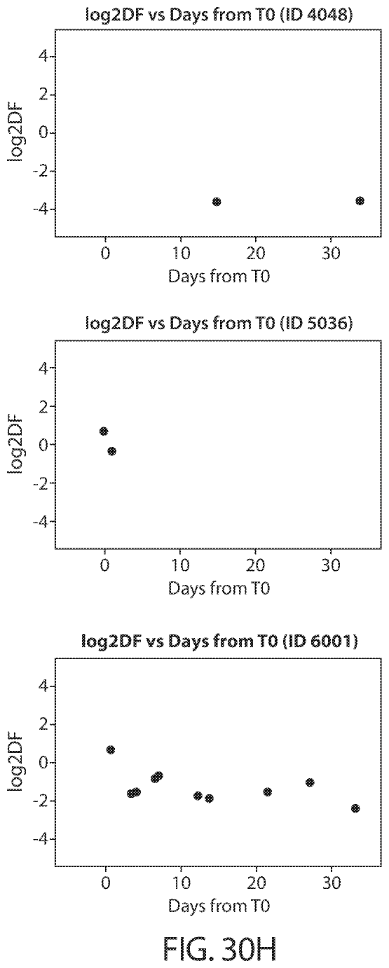

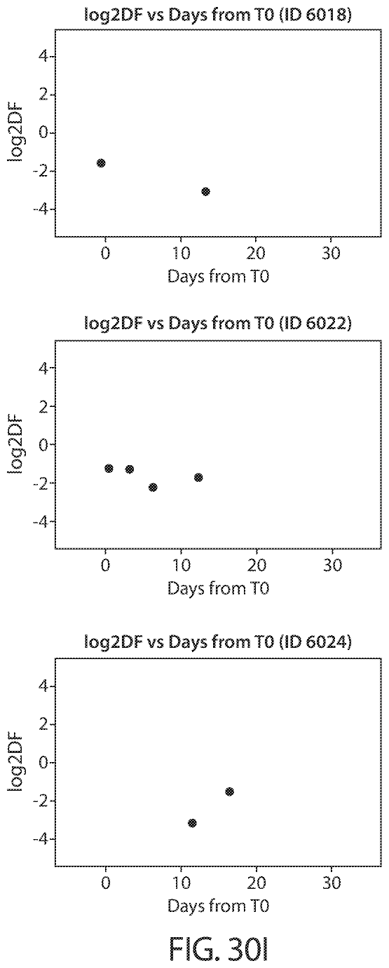

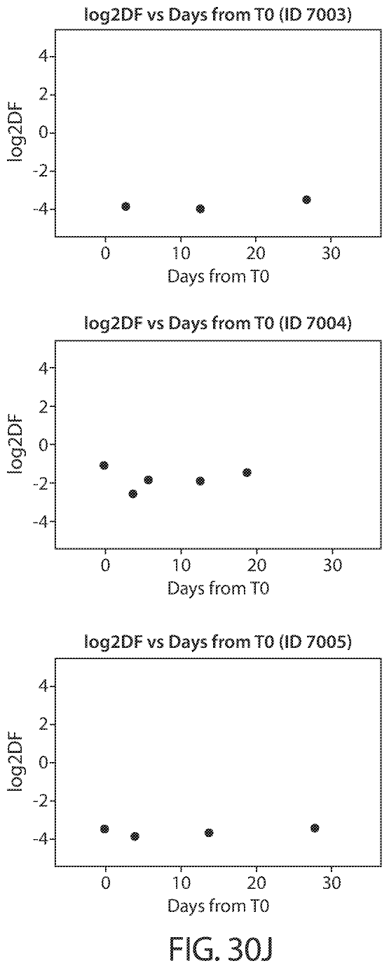

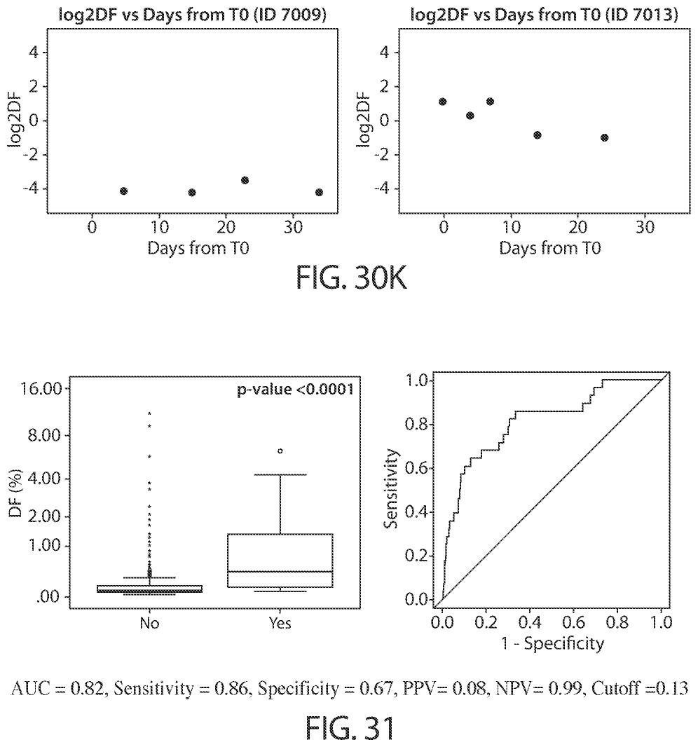

Thirty-two (32) of the 52 subjects were alive and did not experience cardiac arrest (CA) or require MCS (137 total samples from the subjects). Details are provided in the table below, and the plots for the individual subjects are shown in FIGS. 30A-30K.

| Subject ID | #samples |
|---|---|
| 1001 | 12 |
| 1005 | 3 |
| 1006 | 3 |
| 1033 | 9 |
| 1039 | 3 |
| 2003 | 4 |
| 2006 | 2 |
| 2009 | 3 |
| 2011 | 5 |
| 3007 | 2 |
| 3009 | 2 |
| 3012 | 3 |
| 3030 | 2 |
| 3039 | 2 |
| 3049 | 7 |
| 3051 | 6 |
| 3053 | 4 |
| 4009 | 6 |
| 4028 | 3 |
| 4039 | 9 |
| 4042 | 1 |
| 4048 | 2 |
| 5036 | 2 |
| 6001 | 10 |
| 6018 | 2 |
| 6022 | 4 |
| 6024 | 2 |
| 7003 | 3 |
| 7004 | 5 |
| 7005 | 4 |
| 7009 | 4 |
| 7013 | 5 |

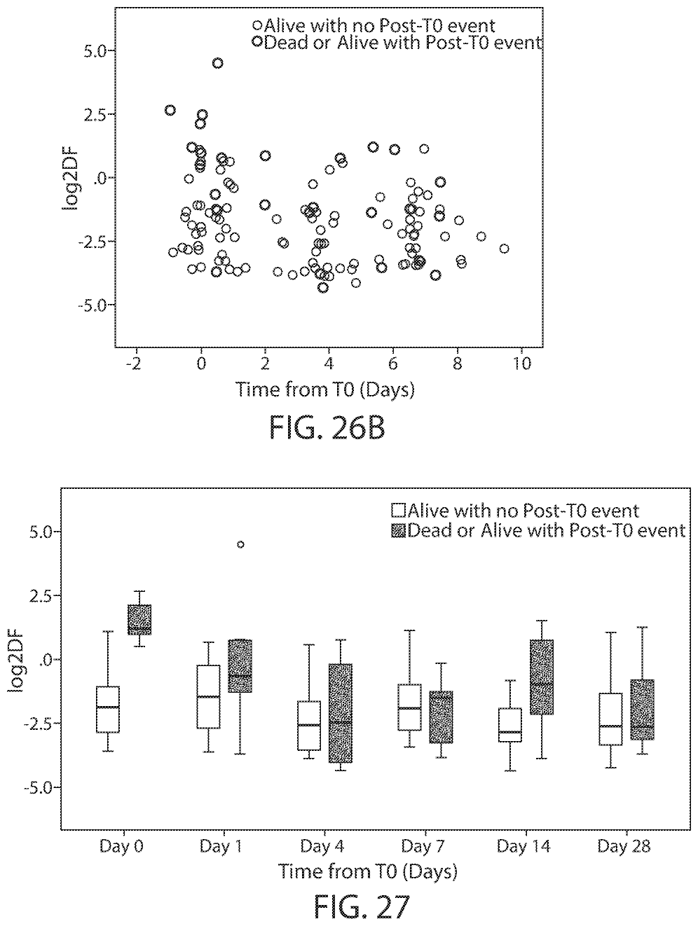

The data was compiled and analyzed further. Differences between log 2 donor-fraction cell-free DNA and status (dead or alive with post-T0 event vs. alive with no post-T0 event) over time were examined. The post-T0 event group included 51 samples from 11 subjects and the no post-T0 event group included 183 samples from 41 subjects. The data is shown in a scatterplot in FIG. 26A and in the table below.

Analysis of GEE Parameter Estimates for log2DF (N = 234)
Empirical Standard Error Estimates

| | | | 95% Confidence | | |
|---|---|---|---|---|---|
| Parameter | | Estimate | Limits | | Pr > \|Z\| |
| Intercept | | −2.2602 | −2.6062 | −1.9142 | <.0001 |
| Post-T0 Event | Yes vs No | 0.9192 | −0.5107 | 2.3492 | 0.2077 |
| Days from T0 | | 0.0086 | 0.0054 | 0.0119 | <.0001 |
| Days from T0* | Yes | −0.0048 | −0.0613 | 0.0517 | 0.8673 |
| Post-T0 Event | | | | | |

The best result obtained using 1-dependent (toeplitz(1)) covariance structure.

The data was also analyzed for samples where the time from T0 was less than or equal to 10 days (125 samples). The post-T0 group event group included 27 samples from nine subjects and the no post-T0 event group included 98 samples from 36 subjects. The data is shown in a scatterplot in FIG. 26B and in the table below.

Analysis of GEE Parameter Estimates for log2DF (N = 125)
Empirical Standard Error Estimates

| | | | 95% Confidence | | |
|---|---|---|---|---|---|
| Parameter | | Estimate | Limits | | Pr > \|Z\| |
| Intercept | | −1.8465 | −2.3122 | −1.3807 | <.0001 |
| Post-T0 Event | Yes vs No | 1.9761 | 0.4670 | 3.4851 | 0.0103 |
| Days from T0 | | −0.0614 | −0.1269 | 0.0041 | 0.0664 |
| Days from T0* | Yes | −0.1736 | −0.4077 | 0.0604 | 0.1460 |
| Post-T0 Event | | | | | |

Best model obtained by AR (1)

Next, different ranges of days after treatment for rejection (T0) were examined. The time points and ranges were defined as follows:
Pre-rejection=Day 0—Within 24 hours prior to onset of treatment for rejection (group=−1)
Day 1—defined as 6-24 hours post initiation of treatment for rejection (group=1)
Day 4—4 days plus or minus 12 hours (group=4)
Day 7—7 days plus or minus 12 hours (group=7)
Day 14—14 days plus or minus 24 hours (group=14)
Day 28—within 7 days of day 28 (Day 21-Day 35) (group=28)

There were 78 samples (out of 234) that did not fall within any of the time windows indicated above. The total number of samples used was 156, as shown in the table below.

| | Frequency (# samples) | | |
|---|---|---|---|
| | Alive with no | Dead or Alive with | |
| | Post-T0 event | Post-T0 event | Total |
| −1 = Day 0 | 17 | 5 | 22 |
| 1 = Day 1 | 16 | 5 | 21 |
| 4 = Day 4 | 15 | 4 | 19 |
| 7 = Day 7 | 19 | 5 | 24 |
| 14 = Day 14 | 21 | 5 | 26 |
| 28 = Day 28 | 36 | 8 | 44 |
| Total | 124 (39 subjects) | 32 (10 subjects) | 156 |

The results are shown in the three tables below and in FIG. 27.

Analysis Variable: log2_df

| | | | Std | | Lower | Upper | | |
|---|---|---|---|---|---|---|---|---|
| group | N | Mean | Dev | Median | Quartile | Quartile | Minimum | Maximum |
| −1 = Day 0 | 22 | −0.9 | 1.9 | −1.2 | −2.7 | 0.6 | −3.6 | 2.6 |
| 1 = Day 1 | 21 | −1.1 | 1.9 | −1.3 | −2.4 | −0.2 | −3.7 | 4.5 |
| 4 = Day 4 | 19 | −2.3 | 1.6 | −2.6 | −3.6 | −1.4 | −4.3 | 0.8 |
| 7 = Day 7 | 24 | −1.8 | 1.3 | −1.8 | −2.9 | −1.0 | −3.8 | 1.1 |
| 14 = Day 14 | 26 | −2.3 | 1.4 | −2.3 | −3.2 | −1.7 | −4.3 | 1.5 |
| 28 = Day 28 | 44 | −2.2 | 1.4 | −2.6 | −3.3 | −1.3 | −4.2 | 1.3 |

| | | Repeated | |
|---|---|---|---|
| | | measure | |
| | | p-values | |
| Consecutive | Parameter | pairwise | |
| Pairs of time | Estimate | (consecutive | FDR |
| windows | for log2DF | pairs) | Adjusted |
| Day 1 vs Day 0 | 0.13 | 0.77 | 0.77 |
| Day 4 vs Day 1 | −1.20 | 0.01 | 0.06 |
| Day 7 vs Day 4 | 0.55 | 0.07 | 0.16 |
| Day 14 vs Day 7 | −0.53 | 0.08 | 0.16 |
| Day 28 vs Day 14 | 0.24 | 0.31 | 0.37 |
| Day 28 vs Day 7 | −0.32 | 0.24 | 0.36 |

Analysis Variable: log2_df

| | Event/No | | | Std | | Lower | Upper | | |
|---|---|---|---|---|---|---|---|---|---|
| group | Event | N | Mean | Dev | Median | Quartile | Quartile | Minimum | Maximum |
| Day 0 | No event | 17 | −1.66 | 1.47 | −1.87 | −2.82 | −1.08 | −3.59 | 1.09 |
| | Event | 5 | 1.48 | 0.87 | 1.18 | 0.97 | 2.11 | 0.51 | 2.65 |
| Day 1 | No event | 16 | −1.46 | 1.41 | −1.47 | −2.69 | −0.24 | −3.60 | 0.66 |
| | Event | 5 | −0.09 | 3.02 | −0.67 | −1.29 | 0.75 | −3.70 | 4.48 |
| Day 4 | No event | 15 | −2.32 | 1.39 | −2.57 | −3.56 | −1.51 | −3.87 | 0.57 |
| | Event | 4 | −2.13 | 2.36 | −2.48 | −4.05 | −0.21 | −4.32 | 0.76 |
| Day 7 | No event | 19 | −1.77 | 1.22 | −1.90 | −2.77 | −0.83 | −3.43 | 1.13 |
| | Event | 5 | −2.00 | 1.51 | −1.51 | −3.26 | −1.24 | −3.82 | −0.17 |
| Day 14 | No event | 21 | −2.68 | 0.98 | −2.84 | −3.22 | −1.90 | −4.35 | −0.82 |
| | Event | 5 | −0.95 | 2.17 | −0.98 | −2.15 | 0.76 | −3.87 | 1.50 |
| Day 28 | No event | 36 | −2.24 | 1.34 | −2.60 | −3.33 | −1.32 | −4.22 | 1.04 |
| | Event | 8 | −1.94 | 1.75 | −2.64 | −3.12 | −0.79 | −3.69 | 1.25 |

Differences were statistically significant at Day 0 and at Day 14:

| | Repeated measure | FDR |
|---|---|---|
| | p-values (Post-T0 | Adjusted |
| | Event Y vs N) | p-values |
| Day 0 | <0.0001 | <0.0001 |
| Day 1 | 0.30 | 0.60 |
| Day 4 | 0.83 | 0.83 |
| Day 7 | 0.68 | 0.82 |
| Day 14 | <0.0001 | <0.0001 |
| Day 28 | 0.68 | 0.82 |

The data used to generate a model for log 2 donor-fraction over time windows for the post-T0 event group:

| | Analysis of GEE Parameter Estimates for log2DF | | | | | |
|---|---|---|---|---|---|---|
| | Empirical Standard Error Estimates | | | | | |
| Parameter | | | Estimate | 95% Confidence Limits | | Pr > \|Z\| |
| Intercept | | | −0.0129 | −1.1093 | 1.0836 | <.0001 |
| group | Day 1 | vs Day 0 | −0.6133 | −2.9567 | 1.7300 | 0.6080 |
| group | Day 4 | | −2.1032 | −3.5805 | −0.6260 | 0.0053 |
| group | Day 7 | | −1.6348 | −3.2514 | −0.0182 | 0.0475 |
| group | Day 14 | | −0.7636 | −2.1693 | 0.6421 | 0.2870 |
| group | Day 28 | | −1.1977 | −2.1521 | −0.2432 | 0.0139 |

The best result obtained using ar(1) covariance structure.

The data used to generate a model for log 2 donor-fraction over time windows for the no post-T0) event group:

Analysis Of GEE Parameter Estimates
Empirical Standard Error Estimates

| Parameter | | Estimate | Estimate | 95% Confidence Limits | | Pr > \|Z\| |
|---|---|---|---|---|---|---|
| Intercept | | | −2.0103 | −2.7009 | −1.3196 | <.0001 |
| group | Day 1 | vs Day 0 | 0.3816 | −0.5939 | 1.3572 | 0.4432 |
| group | Day 4 | | −0.3778 | −1.0987 | 0.3432 | 0.3044 |
| group | Day 7 | | −0.4097 | −1.0824 | 0.2630 | 0.2326 |
| group | Day 14 | | −0.8921 | −1.6309 | −0.1533 | 0.0179 |
| group | Day 28 | | 0.3928 | −0.3153 | 1.1008 | 0.2769 |

The best result obtained using 1-dependent (toeplitz (1)) covariance structure.

### Example 6—Treatment for Rejection and Donor-Specific (Donor-Fraction) Cell-Free DNA

A prospective observational study (DNA-Based Transplant Rejection Test [DTRT]) was conducted among a total of eight established heart transplant centers. This prospective observational study is described here as DTRT-1 where the test assay was developed. DTRT-1 was conducted and enrolled 241 subjects among seven centers. DTRT-1 was followed by a prospective observational study (DTRT-2) which focused on validation of the assay and was conducted and enrolled 147 subjects at seven centers. The combined cohort of the two studies was used to generate combined data. The inclusion and exclusion criteria for the combined cohort are described below. Briefly, subjects who were treated for a rejection and had a test sample collected simultaneously are included. They were excluded if they met the exclusion criteria detailed below. Samples not associated with treatment for rejection were used as the control group excluding those who met the criteria for healthy subject exclusion such as samples within 30 days of diagnosis of coronary vasculopathy or within 14 days of a diagnosis of infection.

Exclusion criteria

| | |
|---|---|
| 1. Clinical | Pre-transplant |
| exclusions | Less than 8 days post-transplant (samples within |
| | 7 days post-transplant) |
| | Pregnancy |
| | Have another transplanted organ, including bone |
| | marrow (anyone with such case) |
| | Have post-transplant lymphoproliferative disease |
| | Have cancer or have had cancer in the previous 2 years |
| | Mechanical Circulatory Support at the time of sample |
| | collection |
| 2. Pre- | TCF, Alu ratio and DF with any quality control failure |
| and post- | |
| Genotype | |
| Quality | |
| Control | |
| exclusions | |

TCF = total cell-free DNA, DF = donor fraction cell-free DNA

Definition: Treatment for rejection was defined as the first change in immunosuppressive therapy with the intention-to-treat suspected or proven allograft rejection on endomyocardial biopsy.

Treatment for rejection was identified as an event by the site investigators and reported to the study. The investigators decided on the designation of the episode based on clinical diagnosis and or on biopsy and therefore reflected the clinical practice. As a part of the primary studies, relevant patient data was prospectively collected. This included demographic information, clinical pre-transplant, transplant and post-transplant information, clinical and laboratory data at follow-up visits as well as events such as rejection, infection, hospitalization, and death. Diagnostic data including catheterization data, biopsy data and imaging data was also collected for scheduled visits as well as for events. The center read and interpretation related to biopsy, echocardiographic and angiography was considered final for the purpose of analysis. A risk-based approach was utilized to monitor the extensive database including one hundred percent monitoring of data fields required for the analysis of primary endpoints and spot monitoring of other data fields.

Details of the study methodology, sample collection, processing and data collection have been previously described. (North et al., 2020; Richmond et al., 2020) The methods for cfDNA and donor fraction analysis are described below.

CfDNA analysis: CfDNA concentration of plasma was measured by quantitative real-time polymerase chain reaction (PCR) targeting the nuclear gene RNase P (Applied Biosystems, Foster City, Calif.) as described. (Hidestrand et al., 2012: North et al., 2020: Richmond et al., 2020) PCR analysis was carried out on an Applied Biosystems QuantStudio 7 Flex Real-Time PCR System (Thermo Fisher Scientific. Waltham, Mass.) or Lightcycler 480 (Roche Applied Science, Penzberg, Germany). A dilution series of commercially sourced human genomic DNA was used to create a standard curve for quantification (Promega, Madison, Wis.). Mean cfDNA concentration in ng/ml plasma for replicates of each sample were determined. To assure pre-analytical quality, a cfDNA fragmentation test to detect leukocyte lysis by comparing short (115 bp) and long (247 bp) multicopy Alu sequences was performed (TAI Diagnostics, Wauwatosa, Wis.). (North et al., 2020)

DF analysis: Donor-specific cfDNA was calculated as a fraction (DF) of the total cfDNA and performed without the requirement of a donor sample (myTAIHEART). (North et al., 2020: Richmond et al., 2020) The test quantitatively genotypes (qGT) a panel of 94 high frequency single-nucleotide polymorphism (SNP) targets selected for the scientist's ability to reliably discriminate between alleles. Briefly, sample cfDNA (15 ng is spiked with an exogenous internal control and amplified by high-fidelity PCR as a multiplexed library followed by qGT analysis where an algorithmic minor species determination of DF using the myTAIHEART software program is performed (TAI Diagnostics, Wauwatosa, Wis.). (North et al., 2020; Richmond et al., 2020) To assure pre-analytical plasma quality, a cfDNA fragmentation test to detect leukocyte lysis by comparing short (115 bp) and long (247 bp) multicopy Alu sequences was performed (TAI Diagnostics, Wauwatosa, Wis.). (North et al., 2020; Richmond et al., 2020)

### Statistical Methods:

### Relationship Between CfDNA and Treatment for Rejection:

The study population was described using appropriate descriptive statistics with continuous measures reported as median and interquartile range. The DF cfDNA was reported as median values with interquartile range. Samples drawn up to 24 hours prior to treatment for rejection were considered Time 0 (T0) samples. If there was more than one treatment within 30 days, then only one T0 (the first one) sample was used for analysis. Relation between the DF cfDNA and outcomes were explored. Generalized Estimating Equation (GEE) (with maximum likelihood estimate) for repeated measures were used (subjects as cluster using AR (1) covariance structures) with logit link function. First and last sample analyses were done using a logistic model. First/Last sample implies first/last sample related to the outcome and first/last healthy sample from rest of the subjects. Significance was set at p<0.05.

Lastly, Receiver Operating Characteristic (ROC) curves were created to assess sensitivity and specificity of DF cfDNA for treatment of rejection. These were separated by age group (pediatric and adult). Alu Ratio threshold=0.50 is used for all results in this report.

### Serial CDNA Testing and Treatment for Rejection:

For analysis assessing relationship between serial testing values of DF cfDNA and outcomes post treatment for rejection, subjects needed to have at least two serial samples associated with the treatment of rejection episodes within 30 days of the episode. Thus, multiple samples per episode were included in this portion of the study (unlike one sample per episode of treatment for rejection in the first part of the study described above). Patient outcomes were divided as alive after treatment for rejection or combined adverse event (cardiac arrest, need for mechanical support or death). For the models, log2DF was used as outcome. Days from T0 and post T0 event were used as covariates. For any negative values for the log (2) transformed DF cfDNA, Gamma distribution was assumed, all values were converted into positive entries by adding a positive number (+5 in this case) in order to include all samples for the model. GEE with Maximum Likelihood Estimation, appropriate covariance structures for the subject clusters and identity link function were used for the model. The covariance structures used to fit the best model (best fit) is reported, log2DF was compared across/within the consecutive time windows using the repeated measures as well. P-values were adjusted using false discovery rate (FDR). An Alu Ratio threshold=0.50 was used for this analysis, consistent with our previous report. (North et al., 2020; Richmond et al., 2020) All analyses were conducted using SAS 9.4 (Cary, N.C.) and R×64 3.6.0.

### Results

The total number of patients enrolled in the two sequential studies was 388 subjects. Based on the inclusion and exclusion criteria described above, a total of 269 subjects were included for this analysis yielding 835 samples. For this cohort, the median age was 15.6 years (range 0-73.4 years) with adults constituting 43% of the study population while the remaining 57% were pediatric patients. Gender distribution showed males were 62.8% of the study group. Race distribution showed White (67%) followed by Black (21.6%) and unknown or not reported (8.5%). These demographic factors are summarized in in the table below.

| | n = 269 |
|---|---|
| Age in year at transplant, Median | 15.6 (0, 73.4) |
| (Range) | |
| Adult, n (%) | 116 (43.1) |
| Male, n (%) | 169 (62.8) |
| Ethnic, n (%) | |
| Hispanic/Latino | 26 (9.7) |
| Non-Hispanic/Latino | 217 (80.7) |
| Unknown | 26 (9.7) |
| Race, n (%) | |
| Black or African American | 58 (21.6) |
| White | 182 (67.7) |
| Asian | 4 (1.5) |
| More than one race | 2 (0.7) |
| Unknown or not reported or other | 23 (8.5) |
| New transplant subjects, n (%) | |
| New | 150 (55.8) |
| Previous | 119 (44.2) |

Of the total 835 samples included, 28 samples were associated with instances of treatment for rejection that met the criteria for inclusion for this analysis. The remainder 807 samples were used as the healthy control group. The distribution of the DF is summarized in the table below. Median DF cfDNA was 0.43 (IQR 0.15, 1.36)% for those with rejection and 0.10 (IQR 0.07, 0.16)% for the controls (p<0.0001) (FIG. 31). Optimized cutoff value of 0.13% was used for the ROC analysis yielding an area under curve (AUC) of 0.82, sensitivity of 0.86, specificity of 0.67, negative predictive value of 0.99 (FIG. 31).

Next, the subjects were separated by age group to assess the performance in pediatric and adult patient populations.

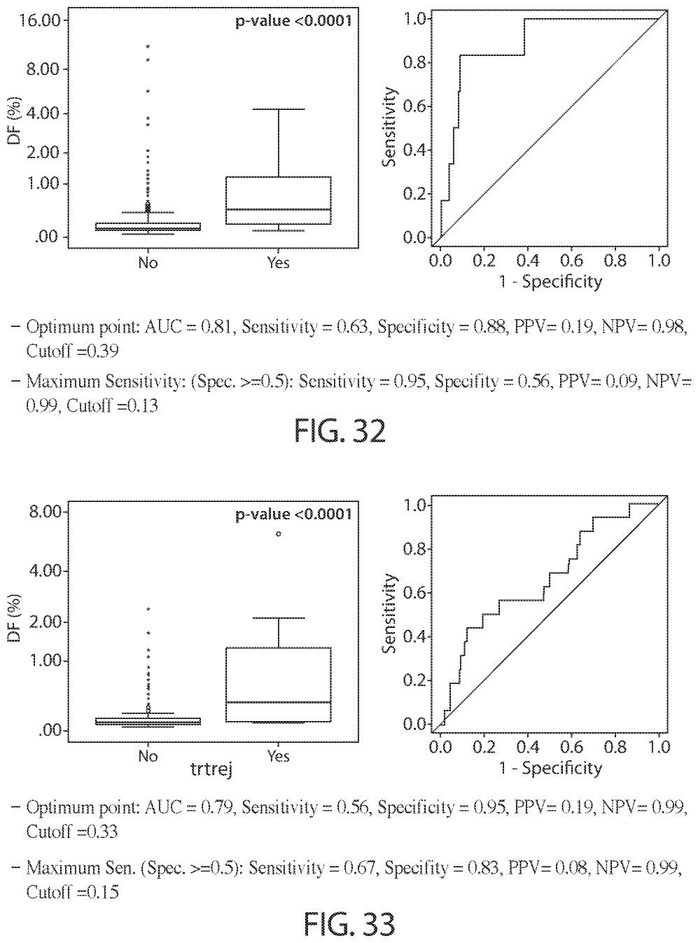

A total of 19 samples were associated with treatment for rejection in pediatric patients (less than 18 years of age). 412 pediatric samples were available as healthy controls. Median DF cfDNA in those treated for rejection was 0.45 (IQR 0.16, 1.42)% for cases and 0.12 (IQR 0.09, 0.20)% for control (p<0.0001) (table below). Optimized cutoff value of 0.13% was used for the ROC analysis yielding an AUC of 0.81, sensitivity of 0.95, specificity of 0.56, negative predictive value of 0.99 (FIG. 32).

| | Treatment | | | |
|---|---|---|---|---|
| | for | | DF (%), median | |
| | rejection | n | (IQR) | P-value |
| All samples | No | 807 | 0.10 (0.07,0.16) | <0.0001 |
| | Yes | 28 | 0.43 (0.15, 1.36) | |
| Pediatric | No | 412 | 0.12 (0.09, 0.20) | <0.0001 |
| samples | Yes | 19 | 0.45 (0.16, 1.42) | |
| Adult samples | No | 395 | 0.09 (0.07, 0.12) | <0.0001 |
| | Yes | 9 | 0.34 (0.09, 1.31) | |
| First samples | No | 242 | 0.11 (0.08, 0.20) | <0.0001 |
| | Yes | 27 | 0.41 (0.14. 1.31) | |
| Pediatric first | No | 135 | 0.13 (0.09, 0.25) | <0.001 |
| sample | Yes | 18 | 0.43 (0.16, 0.96) | |
| Adult first | No | 107 | 0.10 (0.07,0.14) | 0.001 |
| sample | Yes | 9 | 0.34 (0.09, 1.31) | |
| Last samples | No | 242 | 0.10 (0.07.0.14) | <0.0001 |
| | Yes | 27 | 0.41 (0.14, 1.31) | |
| Pediatric last | No | 135 | 0.11 (0.09,0.18) | <0.0001 |
| sample | Yes | 18 | 0.43 (0.16, 0.96) | |
| Adult last | No | 107 | 0.08 (0.06, 0.11) | <0.001 |
| sample | Yes | 9 | 0.34 (0.09, 1.31) | |

DF = donor fraction cell-free DNA

In adult subjects, there were 9 samples associated with treatment for rejection while 395 samples were used as healthy controls. Median DF cfDNA in those treated for rejection was 0.34 (IQR 0.09, 1.31)% for cases and 0.09 (IQR 0.07, 0.12)% for healthy controls (p<0.0001). Optimized cutoff value of 0.15% was used for the ROC analysis yielding an AUC of 0.79, sensitivity of 0.67, specificity of 0.83, negative predictive value of 0.99 (FIG. 33).

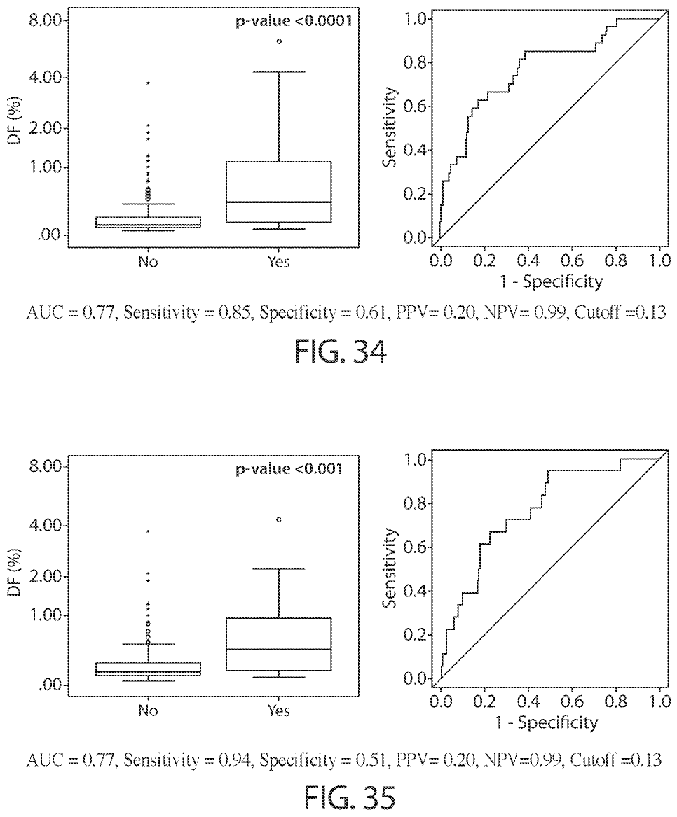

First sample analysis was performed to assess the relationship of first sample associated with treatment for rejection and compared to first sample from healthy subjects. This would account for influence of serial samples in any given patient. Median DF cfDNA in the first samples was 0.41 (IQR 0.14, 1.31)% for cases and 0.11 (IQR 0.08, 0.20)% for healthy controls (p<0.0001)(Table 3). Optimized cutoff value of 0.13% was used for the ROC analysis yielding an AUC of 0.77, sensitivity of 0.85, specificity of 0.61, negative predictive value of 0.97 (FIG. 34).

Separating by age, first sample analysis for pediatric patients showed a median DF cfDNA value of 0.43 (IQR 0.16, 0.96)% for cases and 0.13 (IQR 0.09, 0.25)% for healthy controls (p<0.001). Optimized cutoff value of 0.13% was used for the ROC analysis yielding an AUC of 0.77, sensitivity of 0.94, specificity of 0.51, negative predictive value of 0.99 (FIG. 35).

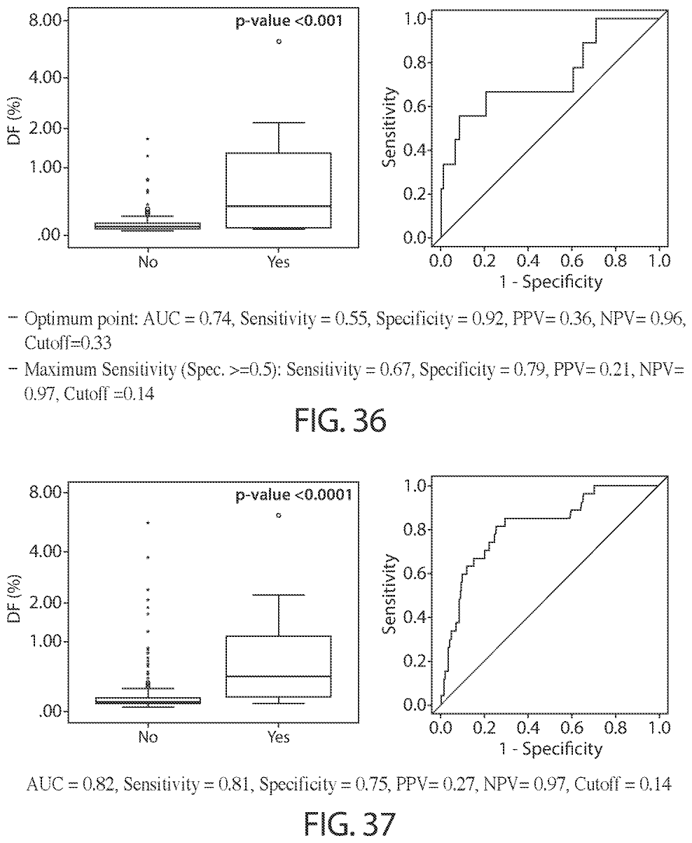

Similarly, for adult patients, first sample analysis showed a median DF cfDNA value of 0.34 (IQR 0.09, 1.31)% for cases and 0.10 (IQR 0.07, 0.14)% for healthy controls (p=0.001). Optimized cutoff value of 0.14% was used for the ROC analysis yielding an AUC of 0.74, sensitivity of 0.67, specificity of 0.79, negative predictive value of 0.97 (FIG. 36).

Last sample analysis was performed using the last sample related to treatment for rejection or last available sample from the healthy subject. The performance was similar, with median DF cfDNA value of 0.41 (IQR 0.14, 1.31)% for cases and 0.10 (IQR 0.07, 0.14)% for healthy controls (p<0.0001). Optimized cutoff value of 0.14% was used for the ROC analysis yielding an AUC of 0.82, sensitivity of 0.81, specificity of 0.75, negative predictive value of 0.97 (FIG. 37).

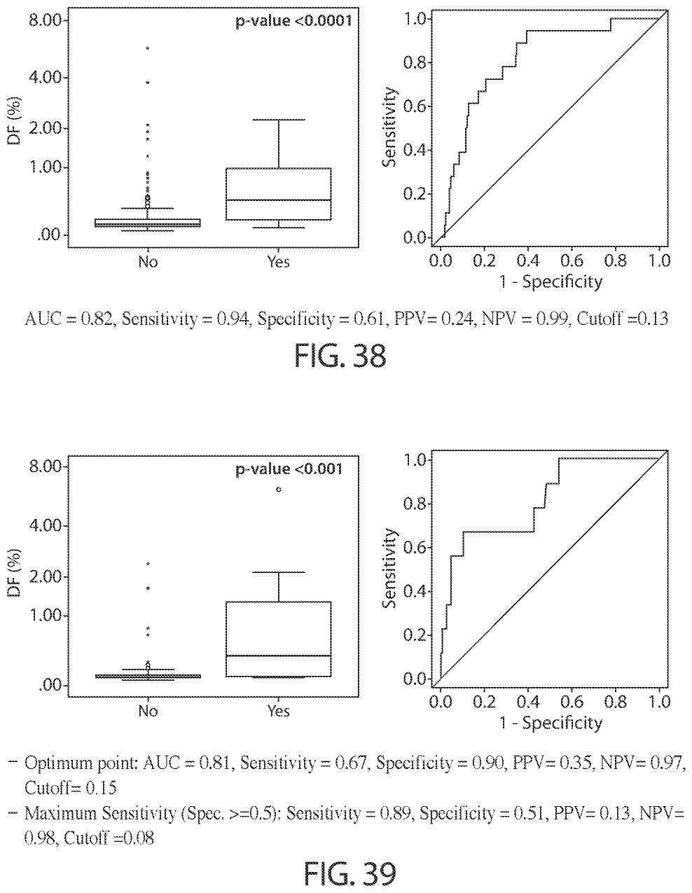

Last sample analysis for pediatric patients showed a median DF cfDNA value of 0.43 (IQR 0.16, 0.96)% for cases and 0.11 (IQR 0.09, 0.18)% for healthy controls (p<0.0001). Optimized cutoff value of 0.13% was used for the ROC analysis yielding an AUC of 0.82, sensitivity of 0.94, specificity of 0.61, negative predictive value of 0.99 (FIG. 38).

Similarly, for adult patients, last sample analysis showed a median DF cfDNA value of 0.34 (IQR 0.09, 1.31)% for cases and 0.08 (IQR 0.06, 0.11)% for healthy controls (p<0.001). Optimized cutoff value of 0.15% was used for the ROC analysis yielding an AUC of 0.81, sensitivity of 0.67, specificity of 0.90, negative predictive value of 0.97. A cutoff of 0.08% resulted in a sensitivity of 0.89, specificity of 0.51 and negative predictive value of 0.98 (FIG. 39).

### Serial Testing and Outcomes:

Second analysis of interest was focused on exploring the relationship between serial values of DF cfDNA after treatment for rejection and the outcomes in those subjects. Two or more samples associated with an episode of treatment for rejection were needed to perform this analysis. Outcomes of interest were death, cardiac arrest, need for mechanical circulatory support after the treatment for rejection. 285 samples from 70 subjects were available for this analysis representing serial collection post treatment for rejection. Of these, 227 samples from 56 subjects were associated with no outcome events after treatment for rejection while 58 samples from 14 subjects were associated with outcome events after treatment for rejection. All these samples were then binned into time windows as follows for purpose of analysis:
Day 0—Within 24 hours prior to onset of treatment for rejection
Day 1—Defined as 6-24 hours post initiation of treatment for rejection
Day 4—4 days plus or minus 12 hours post initiation of treatment for rejection
Day 7—7 days plus or minus 12 hours post initiation of treatment for rejection
Day 14—14 days plus or minus 24 hours post initiation of treatment for rejection
Day 28—Within 7 days of Day 28 (Day 21-Day 35) post initiation of treatment for rejection

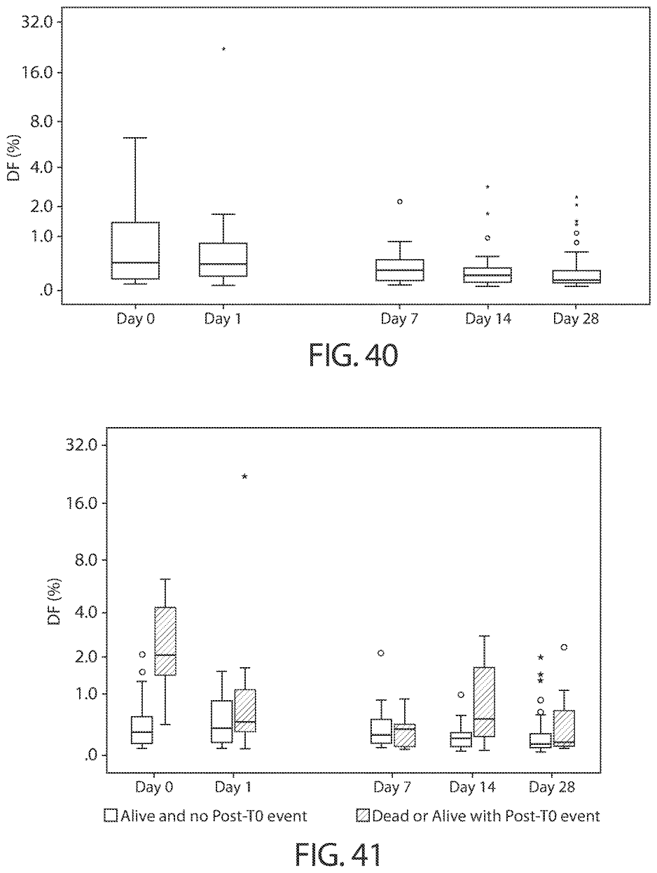

There were 104 samples (out of 285) that were outside of the time windows indicated above and therefore not included in the analysis. Hence a total of 181 samples were used for this analysis (table below). The distribution of the median DF cfDNA values is shown in FIG. 40.

| | Frequency (# samples) | | |
|---|---|---|---|
| | Alive with no | Dead or Alive with | |
| | Post-T0 event | Post-T0 event | Total |
| Day 0 | 20 | 6 | 26 |
| Day 1 | 16 | 7 | 23 |
| Day 4 | 15 | 3 | 18 |
| Day 7 | 19 | 5 | 24 |
| Day 14 | 25 | 5 | 30 |
| =Day 28 | 52 | 8 | 60 |
| Total | 147 (subjects) | 34 (subjects) | 181 |

To assess the sequential change in DF after treatment for rejection, comparison between Day 0 and subsequent values was performed using GEE. In general, the DF cfDNA values decreased in response to treatment for rejection over the following 28 days. There was a statistically significant reduction in the DF at Day 14 and Day 28 compared to Day 0 for treatment for rejection (Table 5).

| | | | | Compared to |
|---|---|---|---|---|
| | | | Compared | Day 0 FDR |
| | | DF (%), median | to Day 0 | adjusted |
| | | (IQR) | p-value | p-value |
| Day 0 | 26 | 0.43 (0.15, 1.42) | — | — |
| Day1 | 23 | 0.41 (0.19, 0.88) | 0.942 | 0.942 |
| Day7 | 24 | 0.29 (0.14, 0.50) | 0.079 | 0.099 |
| Day14 | 30 | 0.23 (0.11, 0.35) | 0.003 | 0.008 |
| Day28 | 60 | 0.13 (0.09, 0.30) | 0.009 | 0.015 |

DF = donor fraction cell-free DNA, FDR = false discovery rate

This trend was then analyzed for those who experienced a post T0 event such as cardiac arrest, need for mechanical support or death compared to those who did not have an event after treatment for rejection. The DF was significantly higher at T0 (at the time of treatment for rejection) for those who had an event thereafter compared to those who did not (a sixfold difference, p<0.0001). There was a significant elevation at Day 14 post treatment for rejection in those who experienced an event compared to those who did not (p<0.0001) (table below). This is graphically shown in the boxplot FIG. 41. There appears to be a relationship between elevated DF at the time of treatment for rejection as well as 14 days after treatment for rejection and events such as cardiac arrest, need for mechanical support or death.

| | Dead or Alive | | | FDR |
|---|---|---|---|---|
| | with Post-T0 | | DF (%), median | adjusted |
| | event | n | (IQR) | p-value |
| Day 0 | No | 20 | 0.31 (0.14, 0.55) | <0.0001 |
| | Yes | 6 | 2.11 (1.42, 4.32) | |
| Day1 | No | 16 | 0.36 (0.16, 0.85) | 0.64 |
| | Yes | 7 | 0.46 (0.20, 1.69) | |
| Day7 | No | 19 | 0.27 (0.15, 0.56) | 0.69 |
| | Yes | 5 | 0.35 (0.10, 0.42) | |
| Day14 | No | 25 | 0.22 (0.11, 0.29) | <0.0001 |
| | Yes | 5 | 0.51 (0.23, 1.69) | |
| Day28 | No | 52 | 0.13 (0.09, 0.28) | 0.64 |
| | Yes | 8 | 0.16 (0.12, 0.70) | |

DF = donor fraction cell-free DNA, FDR = false discovery rate

### Discussion

The study demonstrates the utility of DF cfDNA in assessing clinical rejection in pediatric and adult patients with heart transplantation. DF cfDNA is able to predict absence of clinical rejection with a high degree of confidence (negative predictive value of 99%). It has diagnostic value for assessment of rejection in both pediatric and adult heart transplant patients with an AUC of 0.82. Additionally, elevated levels of DF cfDNA during serial monitoring were able to predict important clinical outcomes after an episode of rejection such as need for mechanical circulatory support or death.

In contrast to published studies, this study is a representative sample of real-life clinical patients as this study population was not pre-selected for low risk. A serial measurement of donor-derived cfDNA may provide a better predictor of response to therapy and outcomes after an episode of rejection. In this Example, donor-derived cfDNA at the time of a treated episode of rejection as well as at Day 14 after such an episode predicted important outcomes such as cardiac arrest, mechanical support or death. A 14 day window is important as it corresponds with a common practice of performing follow-up biopsy two weeks post-rejection episode. Those with an increase at the 14 day mark may represent a cohort of patients that were only partially responsive to the acute therapies and have an ongoing injury to the graft.

In analysis of a large cohort of prospectively enrolled heart transplant patients, it was found that the DF cfDNA can be used to screen patients for clinically significant rejection. The assay has good specificity, sensitivity, and negative predictive value. Additionally, serial measurement of DF cfDNA may predict clinically important outcomes such as cardiac arrest and death in patients treated for rejection.

### REFERENCES

AGBOR-ENOH, S. et al. Cell-Free DNA to Detect Heart Allograft Acute Rejection. Circulation, Jan. 13, 2021. ISSN 0009-7322.
ANGELINI, A. et al. A web-based pilot study of inter-pathologist reproducibility using the ISHLT 2004 working formulation for biopsy diagnosis of cardiac allograft rejection: the European experience. J Heart Lung Transplant, v. 30, n. 11, p. 1214-20, November 2011. ISSN 1557-3117 (Electronic) 1053-2498 (Linking). Available at: <https://www.ncbi.nlm.nih.gov/pubmed/21816625>.
BERRY, G. J. et al. The ISHLT working formulation for pathologic diagnosis of antibody-mediated rejection in heart transplantation: Evolution and current status (2005-2011). Journal of Heart and Lung Transplantation, v. 30, n. 6, p. 601-611, June 2011. ISSN 1053-2498. Available at: <<Go to ISI>://WOS:000290834600001>.
BIENKOWSKI, M. et al. Liquid biopsy for minimally invasive heart transplant monitoring: a pilot study. J Clin Pathol, v. 73, n. 8, p. 507-510, August 2020. ISSN 1472-4146 (Electronic) 0021-9746 (Linking). Available at: <https://www.ncbi.nlm.nih.gov/pubmed/31806731>. Available at: <https://jcp.bmj.com/content/73/8/507.long>.
CRESPO-LEIRO, M. G. et al. Concordance Among Pathologists in the Second Cardiac Allograft Rejection Gene Expression Observational Study (CARGO II). Transplantation Journal, v. 94, n. 11, p. 1172-1177, Available at: <http://dx.doi.org/10.1097/tp.0b013e31826e19e2>.
DANDEL, M.; HETZER, R. Non-invasive cardiac allograft rejection surveillance: reliability and clinical value for prevention of heart failure. Heart Fail Rev, Sep. 5, 2020. ISSN 1573-7322 (Electronic) 1382-4147 (Linking). Available at: <https://www.ncbi.nlm.nih.gov/pubmed/32889634>. Available at: <https://link.springer.com/article/10.1007%2Fs10741-020-10023-3>.
DENG, M. C. et al. Noninvasive discrimination of rejection in cardiac allograft recipients using gene expression profiling. Am J Transplant, v. 6, n. 1, p. 150-60, January 2006. ISSN 1600-6135 (Print) 1600-6135 (Linking). Available at: <https://www.ncbi.nlm.nih.gov/pubmed/16433769>. Available at: <https://onlinelibrary.wiley.com/doi/pdfdirect/10.1111/j.1600-6143.2005.01175.x?download=true>.
HIDESTRAND, M. et al. Influence of temperature during transportation on cell-free DNA analysis. Fetal Diagn Ther, v. 31, n. 2, p. 122-8, 2012. ISSN 1421-9964 (Electronic) 1015-3837 (Linking). Available at: <https://www.ncbi.nlm.nih.gov/pubmed/22261730>. Available at: <https://www.karger.com/Article/Abstract/335020>.
KHUSH, K. K. et al. The International Thoracic Organ Transplant Registry of the International Society for Heart and Lung Transplantation: Thirty-sixth adult heart transplantation report—2019; focus theme: Donor and recipient size match. J Heart Lung Transplant, v. 38, n. 10, p. 1056-1066, October 2019. ISSN 1557-3117 (Electronic) 1053-2498 (Linking). Available at: <https://www.ncbi.nlm.nih.gov/pubmed/31548031>.
Noninvasive detection of graft injury after heart transplant using donor-derived cell-free DNA: A prospective multicenter study. American journal of transplantation, v. 19, n. 10, p. 2889-2899, 2019. ISSN 1600-6135. Available at: <https://www.ncbi.nlm.nih.gov/pmc/articles/PMC6790566/pdf/AJT-19-2889.pdf>.
MANUAL., P. H. T. S. D. D. https://www.uab.edu/medicine/phts/images/2015_Forms/2020-10-20/Version_1.1.14_Manual_FINAL-1.pdf. Accessed 03/12/2021.
MENGEL, M. et al. The molecular phenotype of heart transplant biopsies: relationship to histopathological and clinical variables. Am J Transplant, v. 10, n. 9, p. 2105-15, September 2010. ISSN 1600-6143 (Electronic) 1600-6135 (Linking). Available at: <https://www.ncbi.nlm.nih.gov/pubmed/20883545>. Available at: <https://onlinelibrary.wiley.com/doi/pdfdirect/10.1111/j.1600-6143.2010.03182.x?download=true>.
NORTH, P. E. et al. Cell-free DNA donor fraction analysis in pediatric and adult heart transplant patients by multiplexed allele-specific quantitative PCR: Validation of a rapid and highly sensitive clinical test for stratification of rejection probability. PLoS One, v. 15, n. 1, p. e0227385, 2020. ISSN 1932-6203 (Electronic) 1932-6203 (Linking). Available at: <https://www.ncbi.nlm.nih.gov/pubmed/31929557>. Available at: <https://www.ncbi.nlm.nih.gov/pmc/articles/PMC6957190/pdf/pone.0227385.pdf>.
PAHL, E. et al. Death after rejection with severe hemodynamic compromise in pediatric heart transplant recipients: a multi-institutional study. J Heart Lung Transplant, v. 20, n. 3, p. 279-87, March 2001. ISSN 1053-2498 (Print) 1053-2498 (Linking). Available at: <https://www.ncbi.nlm.nih.gov/pubmed/11257553>. Available at: <https://www.sciencedirect.com/science/article/abs/pii/S105324980000228X?via%3Dihub>.
PEYSTER, E. G.; MADABHUSHI, A.; MARGULIES, K. B. Advanced Morphologic Analysis for Diagnosing Allograft Rejection: The Case of Cardiac Transplant Rejection. Transplantation, v. 102, n. 8, p. 1230-1239, August 2018. ISSN 1534-6080 (Electronic) 0041-1337 (Linking). Available at: <https://www.ncbi.nlm.nih.gov/pubmed/29570167>. Available at: <https://www.ncbi.nlm.nih.gov/pmc/articles/PMC6059998/pdf/tp-102-1230.pdf>.
PHAM, M. X. et al. Gene-expression profiling for rejection surveillance after cardiac transplantation. N Engl J Med, v. 362, n. 20, p. 1890-900, May 20, 2010. ISSN 1533-4406 (Electronic) 0028-4793 (Linking). Available at: <https://www.ncbi.nlm.nih.gov/pubmed/20413602>. Available at: <https://www.nejm.org/doi/pdf/10.1056/NEJMoa0912965?articleTools=true>.
RICHMOND. M. E. et al. Donor fraction cell-free DNA and rejection in adult and pediatric heart transplantation. J Heart Lung Transplant, v. 39, n. 5, p. 454463, May 2020. ISSN 1557-3117 (Electronic) 1053-2498 (Linking). Available at: <https://www.ncbi.nlm.nih.gov/pubmed/31983667>.
SINGH, T. P.; GAUVREAU, K. Center effect on posttransplant survival among currently active United States pediatric heart transplant centers. Am J Transplant, v. 18, n. 12, p. 2914-2923, December 2018. ISSN 1600-6143 (Electronic) 1600-6135 (Linking). Available at: <https://www.ncbi.nlm.nih.gov/pubmed/29806728>.
STEWART, S. et al. Revision of the 1990 Working Formulation for the Standardization of Nomenclature in the Diagnosis of Heart Rejection. The Journal of Heart and Lung Transplantation, v. 24, n. 11, p. 1710-1720, 2005. Available at: <http://dx.doi.org/10.1016/j.healun.2005.03.019>. Available at: <https://www.sciencedirect.com/science/article/abs/pii/S1053249805002032?via%3Dihub>.
XIN, A. et al. Identifying low-grade cellular rejection after heart transplantation in children by using gene expression profiling. Physiol Genomics, v. 50, n. 3, p. 190-196, Mar. 1, 2018. ISSN 1531-2267 (Electronic) 1094-8341 (Linking). Available at: <https://www.ncbi.nlm.nih.gov/pubmed/29341866>.
YANG, H. M. et al. Has the 2004 revision of the International Society of Heart and Lung Transplantation grading system improved the reproducibility of the diagnosis and grading of cardiac transplant rejection? Cardiovasc Pathol, v. 18, n. 4, p. 198-204. July-August 2009. ISSN 1879-1336 (Electronic) 1054-8807 (Linking). Available at: <https://www.ncbi.nlm.nih.gov/pubmed/18619859>.
ZANGWILL, S. D. et al. Effect of endomyocardial biopsy on levels of donor-specific cell-free DNA. The Journal of Heart and Lung Transplantation, v. 38, n. 10, p. 1118-1120, Available at: <http://dx.doi.org/10.1016/j.healun.2019.06.005>.
ZUPPAN, C. W. et al. Cause of death in pediatric and infant heart transplant recipients: review of a 20-year, single-institution cohort. The Journal of Heart and Lung Transplantation, v. 28, n. 6, p. 579-584, 2009. ISSN 1053-2498.

What is claimed is:

1. A method of assessing a sample from a transplant subject, the method comprising:
(a) determining an amount of donor-specific cell-free DNA (DS cf-DNA) in a sample taken from the subject at or before a treatment for transplant risk (e.g., day 0) and/or during treatment for transplant risk (e.g., day 14); and
(b) comparing the amount(s) of DS cf-DNA to a threshold value, such as 0.13, 0.14, 0.23, or 0.43, or any one of the values of the below table, e.g., for day 0 and/or day 14, respectively, such as the mean, median, lower quartile, etc. of the table, respectively, wherein when the amount(s) is greater than or equal to the threshold value risk, such as rejection, is indicated or increased.
Analysis Variable: log2_df

| | Event/No | | | Std | | Lower | Upper | | |
|---|---|---|---|---|---|---|---|---|---|
| group | Event | N | Mean | Dev | Median | Quartile | Quartile | Minimum | Maximum |
| Day 0 | No event | 17 | −1.66 | 1.47 | −1.87 | −2.82 | −1.08 | −3.59 | 1.09 |
| | Event | 5 | 1.48 | 0.87 | 1.18 | 0.97 | 2.11 | 0.51 | 2.65 |
| Day 1 | No event | 16 | −1.46 | 1.41 | −1.47 | −2.69 | −0.24 | −3.60 | 0.66 |
| | Event | 5 | −0.09 | 3.02 | −0.67 | −1.29 | 0.75 | −3.70 | 4.48 |
| Day 4 | No event | 15 | −2.32 | 1.39 | −2.57 | −3.56 | −1.51 | −3.87 | 0.57 |
| | Event | 4 | −2.13 | 2.36 | −2.48 | −4.05 | −0.21 | −4.32 | 0.76 |
| Day 7 | No event | 19 | −1.77 | 1.22 | −1.90 | −2.77 | −0.83 | −3.43 | 1.13 |
| | Event | 5 | −2.00 | 1.51 | −1.51 | −3.26 | −1.24 | −3.82 | −0.17 |
| Day 14 | No event | 21 | −2.68 | 0.98 | −2.84 | −3.22 | −1.90 | −4.35 | −0.82 |
| | Event | 5 | −0.95 | 2.17 | −0.98 | −2.15 | 0.76 | −3.87 | 1.50 |
| Day 28 | No event | 36 | −2.24 | 1.34 | −2.60 | −3.33 | −1.32 | −4.22 | 1.04 |
| | Event | 8 | −1.94 | 1.75 | −2.64 | −3.12 | −0.79 | −3.69 | 1.25 |

2. A method of assessing a sample from a transplant subject, the method comprising:
(a) determining an amount of donor-specific cell-free DNA (DS cf-DNA) in a sample taken from the subject; and
(b) comparing the amount of DS cf-DNA to a threshold value of 0.13 or 0.14 or any one of the values of the table, wherein when the amount is greater than or equal to the threshold value risk, such as rejection, is indicated.

3. The method of claim 1 or claim 2, wherein the method further comprises:
(c) reporting and/or recording the amount(s) of DS cf-DNA.

4. A method of assessing a transplant subject, the method comprising:
(a) obtaining an amount of donor-specific cell-free DNA (DS cf-DNA) in a sample taken from the subject at or before a treatment for transplant risk (e.g., day 0) and/or during treatment for transplant risk (e.g., day 14); and
(b) comparing the amount(s) of DS cf-DNA to a threshold value, such as 0.13, 0.14, 0.23, or 0.43, or any one of the values of the below table, e.g., for day 0 and/or day 14, respectively, such as the mean, median, lower quartile, etc. of the table, respectively, wherein when the amount(s) is greater than or equal to the threshold value risk, such as rejection, is indicated or increased; and
(c) determining a treatment or monitoring regimen for the subject based on the comparison(s) and/or making a treatment management decision for the subject.
Analysis Variable: log2_df

| | Event/No | | | Std | | Lower | Upper | | |
|---|---|---|---|---|---|---|---|---|---|
| group | Event | N | Mean | Dev | Median | Quartile | Quartile | Minimum | Maximum |
| Day 0 | No event | 17 | −1.66 | 1.47 | −1.87 | −2.82 | −1.08 | −3.59 | 1.09 |
| | Event | 5 | 1.48 | 0.87 | 1.18 | 0.97 | 2.11 | 0.51 | 2.65 |
| Day 1 | No event | 16 | −1.46 | 1.41 | −1.47 | −2.69 | −0.24 | −3.60 | 0.66 |
| | Event | 5 | −0.09 | 3.02 | −0.67 | −1.29 | 0.75 | −3.70 | 4.48 |
| Day 4 | No event | 15 | −2.32 | 1.39 | −2.57 | −3.56 | −1.51 | −3.87 | 0.57 |
| | Event | 4 | −2.13 | 2.36 | −2.48 | −4.05 | −0.21 | −4.32 | 0.76 |
| Day 7 | No event | 19 | −1.77 | 1.22 | −1.90 | −2.77 | −0.83 | −3.43 | 1.13 |
| | Event | 5 | −2.00 | 1.51 | −1.51 | −3.26 | −1.24 | −3.82 | −0.17 |
| Day 14 | No event | 21 | −2.68 | 0.98 | −2.84 | −3.22 | −1.90 | −4.35 | −0.82 |
| | Event | 5 | −0.95 | 2.17 | −0.98 | −2.15 | 0.76 | −3.87 | 1.50 |
| Day 28 | No event | 36 | −2.24 | 1.34 | −2.60 | −3.33 | −1.32 | −4.22 | 1.04 |
| | Event | 8 | −1.94 | 1.75 | −2.64 | −3.12 | −0.79 | −3.69 | 1.25 |

5. A method of assessing a transplant subject, the method comprising:
(a) obtaining an amount of donor-specific cell-free DNA (DS cf-DNA); and
(b) comparing the amount of DS cf-DNA to a threshold value of 0.13 or 0.14 or any one of the values of the above table, wherein when the amount is greater than or equal to the threshold value risk, such as rejection, is indicated; and
(c) determining a treatment or monitoring regimen for the subject based on the comparison.

6. The method of any one of claims 1-5, wherein one or more further amounts of DS cf-DNA are obtained from a sample taken from the subject at a different point in time.

7. The method of claim 6, wherein the one or more further amounts of DS cf-DNA are obtained from samples taken from the subject daily, weekly, monthly, or bimonthly.

8. The method of any one of the preceding claims, wherein the amounts are provided in a report.

9. A report of that comprises the amounts of any one of claims 1-7.

10. The method of any one of the preceding claims, wherein the amounts are recorded in a database.

11. A database that comprises the amounts of any one of claims 1-7.

12. The method of any one of any one of the preceding claims, wherein the determining a monitoring regimen comprises determining the amount of DS cf-DNA in the subject over time or at a subsequent point in time, or suggesting such monitoring to the subject.

13. The method of any one of any one of the preceding claims, wherein the time between samples is decreased if the amount of DS cf-DNA is increased relative to threshold(s) or amount(s) from earlier time point(s).

14. The method of any one of the preceding claims, wherein the determining a monitoring regimen comprises using or suggesting the use of one or more additional test(s) to assess the subject.

15. The method of any one of the preceding claims, wherein the determining a treatment regimen comprises selecting or suggesting a treatment for the subject or changing the treatment of the subject or suggesting such change.

16. The method of any one of the preceding claims, wherein the determining a treatment regimen comprises treating the subject.

17. The method of any one of the preceding claims, wherein the determining a treatment regimen comprises providing information about a treatment to the subject.

18. The method of any one of the preceding claims, wherein the making a treatment management decision comprises determining that additional testing and/or monitoring is required, initiating a treatment, changing the frequency of a treatment, changing the dosage of the treatment, changing the frequency and/or dosage of the treatment, changing the type of treatment to be performed, changing the timing of the treatment, or any combination of the foregoing.

19. The method of any one of the preceding claims, wherein the sample is a blood, plasma or serum sample.

20. The method of claim 19, wherein the blood sample is a plasma sample.

21. The method of any one of the preceding claims, wherein the transplant subject is a heart transplant subject, such as an adult or a pediatric heart transplant subject.

22. A method of assessing a sample from a transplant subject treated for rejection, the method comprising:
(a) determining an amount of donor-specific cell-free DNA (DS cf-DNA) in at least one sample taken from the subject, wherein the at least one sample is taken prior to rejection treatment or at the start of such treatment (e.g., day 0) and/or taken post treatment (e.g., day 14); and
(b) comparing the amount(s) to a threshold, respectively, such as 0.13, 0.14, 0.23, or 0.43, or any one of the values of the below table, e.g., for day 0 and/or day 14, such as the mean, median, lower quartile, etc. of the table, respectively, to assess a risk in the subject, wherein when the amount(s) are greater than or equal to the threshold value risk is indicated or increased.
Analysis Variable: log2_df

| | Event/No | | | Std | | Lower | Upper | | |
|---|---|---|---|---|---|---|---|---|---|
| group | Event | N | Mean | Dev | Median | Quartile | Quartile | Minimum | Maximum |
| Day 0 | No event | 17 | −1.66 | 1.47 | −1.87 | −2.82 | −1.08 | −3.59 | 1.09 |
| | Event | 5 | 1.48 | 0.87 | 1.18 | 0.97 | 2.11 | 0.51 | 2.65 |
| Day 1 | No event | 16 | −1.46 | 1.41 | −1.47 | −2.69 | −0.24 | −3.60 | 0.66 |
| | Event | 5 | −0.09 | 3.02 | −0.67 | −1.29 | 0.75 | −3.70 | 4.48 |
| Day 4 | No event | 15 | −2.32 | 1.39 | −2.57 | −3.56 | −1.51 | −3.87 | 0.57 |
| | Event | 4 | −2.13 | 2.36 | −2.48 | −4.05 | −0.21 | −4.32 | 0.76 |
| Day 7 | No event | 19 | −1.77 | 1.22 | −1.90 | −2.77 | −0.83 | −3.43 | 1.13 |
| | Event | 5 | −2.00 | 1.51 | −1.51 | −3.26 | −1.24 | −3.82 | −0.17 |
| Day 14 | No event | 21 | −2.68 | 0.98 | −2.84 | −3.22 | −1.90 | −4.35 | −0.82 |
| | Event | 5 | −0.95 | 2.17 | −0.98 | −2.15 | 0.76 | −3.87 | 1.50 |
| Day 28 | No event | 36 | −2.24 | 1.34 | −2.60 | −3.33 | −1.32 | −4.22 | 1.04 |
| | Event | 8 | −1.94 | 1.75 | −2.64 | −3.12 | −0.79 | −3.69 | 1.25 |

23. The method of claim 22, wherein the method further comprises:
(c) reporting and/or recording the amount(s) of DS cf-DNA.

24. The method of claim 23, wherein the method further comprises:
(d) comparing the amount(s) of DS cf-DNA to a threshold value, such as 0.13, 0.14, 0.23, or 0.43, or any one of the values of the below table, e.g., for day 0 and/or day 14, respectively, such as the mean, median, lower quartile, etc. of the table, respectively, wherein when the amount(s) are greater than or equal to the threshold value risk is indicated or increased.
Analysis Variable: log2_df

| | Event/No | | | Std | | Lower | Upper | | |
|---|---|---|---|---|---|---|---|---|---|
| group | Event | N | Mean | Dev | Median | Quartile | Quartile | Minimum | Maximum |
| Day 0 | No event | 17 | −1.66 | 1.47 | −1.87 | −2.82 | −1.08 | −3.59 | 1.09 |
| | Event | 5 | 1.48 | 0.87 | 1.18 | 0.97 | 2.11 | 0.51 | 2.65 |
| Day 1 | No event | 16 | −1.46 | 1.41 | −1.47 | −2.69 | −0.24 | −3.60 | 0.66 |
| | Event | 5 | −0.09 | 3.02 | −0.67 | −1.29 | 0.75 | −3.70 | 4.48 |
| Day 4 | No event | 15 | −2.32 | 1.39 | −2.57 | −3.56 | −1.51 | −3.87 | 0.57 |
| | Event | 4 | −2.13 | 2.36 | −2.48 | −4.05 | −0.21 | −4.32 | 0.76 |
| Day 7 | No event | 19 | −1.77 | 1.22 | −1.90 | −2.77 | −0.83 | −3.43 | 1.13 |
| | Event | 5 | −2.00 | 1.51 | −1.51 | −3.26 | −1.24 | −3.82 | −0.17 |
| Day 14 | No event | 21 | −2.68 | 0.98 | −2.84 | −3.22 | −1.90 | −4.35 | −0.82 |
| | Event | 5 | −0.95 | 2.17 | −0.98 | −2.15 | 0.76 | −3.87 | 1.50 |
| Day 28 | No event | 36 | −2.24 | 1.34 | −2.60 | −3.33 | −1.32 | −4.22 | 1.04 |
| | Event | 8 | −1.94 | 1.75 | −2.64 | −3.12 | −0.79 | −3.69 | 1.25 |

25. A method of assessing a sample from a transplant subject treated for rejection, the method comprising:
(a) determining an amount of donor-specific cell-free DNA (DS cf-DNA) in at least one sample taken from the subject, wherein the at least one sample is taken prior to rejection treatment and/or taken post treatment; and
(b) comparing the amount(s) to threshold of 0.13 or 0.14 or any one of the values of the above tables to assess a risk in the subject, wherein when the amount(s) are greater than or equal to the threshold value risk is indicated or increased.

26. The method of claim 25, wherein the method further comprises:
(c) reporting and/or recording the amount(s) of DS cf-DNA.

27. The method of claim 26, wherein the method further comprises:
(d) comparing the amount(s) of DS cf-DNA to a threshold value of 0.13 or 0.14 or any one of the values of the above tables, wherein when the amount(s) are greater than or equal to the threshold value risk is indicated or increased.

28. The method of any one of claims 22-27, wherein at least one sample is taken prior to treatment such as immediately prior to the treatment.

29. The method of any one of claims 22-28, wherein one or more further amounts of DS cf-DNA are determined each from a sample taken from the subject at a different point in time, such as a different point in time during the treatment for the rejection.

30. The method of any one of claims 22-29, wherein the method further comprises:
(f) determining a treatment or monitoring regimen or making a treatment management decision for the subject based on the amount(s) of DS cf-DNA compared to the threshold values.

31. The method of any one of claims 22-30, wherein the amount(s) are provided in a report.

32. A report of that comprises the amount(s) of any one of claims 22-31.

33. The method of any one of claims 22-31, wherein the amount(s) are recorded in a database.

34. A database that comprises the amount(s) of any one of claims 22-31.

35. The method of any one of claims 22-31, wherein an amount of DS cf-DNA that is greater than a threshold value represents an increased risk.

36. The method of any one of claims 22-31, wherein an amount of DS cf-DNA that is lower than a threshold value represents a decreased risk.

37. The method of any one of claims 22-36, wherein the determining a monitoring regimen comprises determining the amount of DS cf-DNA in the subject over time or at a subsequent point in time, or suggesting such monitoring to the subject.

38. The method of any one of claims 22-36, wherein the time between samples is decreased if the amount of DS cf-DNA is increased relative to the threshold value.

39. The method of any one of claims 22-38, wherein the determining a monitoring regimen comprises using or suggesting the use of one or more additional test(s) to assess the subject.

40. The method of any one of claims 22-38, wherein the determining a treatment regimen comprises selecting or suggesting a treatment for the subject or changing the treatment of the subject or suggesting such change.

41. The method of any one of claims 22-40, wherein the determining a treatment regimen comprises treating the subject.

42. The method of any one of claims 22-40, wherein the determining a treatment regimen comprises providing information about a treatment to the subject.

43. The method of any one of claims 22-42, wherein the making a treatment management decision comprises determining that additional testing and/or monitoring is required, initiating a treatment, changing the frequency of a treatment, changing the dosage of the treatment, changing the frequency and/or dosage of the treatment, changing the type of treatment to be performed, changing the timing of the treatment, or any combination of the foregoing

44. The method of any one of claims 22-43, wherein the sample is a blood, plasma or serum sample.

45. The method of claim 44, wherein the blood sample is a plasma sample.

46. The method of any one of claims 22-45, wherein the transplant subject is a heart transplant subject, such as an adult or a pediatric heart transplant subject.

47. The method of any one of claims 22-46, wherein the subject has been treated for rejection at least once.

48. The method of any one of the preceding claims, wherein the subject is any one of the subjects provided herein or is one that has or is suspected of having any one of the conditions provided herein.

49. The method of any one of the preceding claims, wherein the treatment is any one of the treatments provided herein based on the determination or comparison.

50. The method of any one of the preceding claims, wherein the method further comprises treating the subject or suggesting a treatment to the subject.

51. The method of claim 35 or claim 36, wherein the risk is risk of transplant rejection, cardiac arrest, required mechanical circulatory support, and/or death.
